SEQUENCE LISTING (22 sequences)

SEQ ID NO: 1 - DNA, 21 bp, Unknown
  primer_bind: Forward Primer Collagen
agacttgcgtctaccccaatc

SEQ ID NO: 2 - DNA, 20 bp, Unknown
  primer_bind: Forward Primer Collagen
gcaggcgtaggaaggtcatc

SEQ ID NO: 3 - DNA, 21 bp, Unknown
  CDS: Forward Primer ACAN Protein Coding Gene
cacgatgcctttcaccacgac

SEQ ID NO: 4 - DNA, 21 bp, Unknown
  CDS: Reverse Primer ACAN Protein Coding Gene
tgcgggtcaacagtgcctatc

SEQ ID NO: 5 - DNA, 21 bp, Unknown
  CDS: Forward Primer SOX9 Protein Coding Gene
gctctggagacttctgaacga

SEQ ID NO: 6 - DNA, 20 bp, Unknown
  CDS: Reverse Primer SOX9 Protein Coding Gene
ccgttcttcaccgacttcct

SEQ ID NO: 7 - DNA, 20 bp, Unknown
  CDS: Forward Primer Transforming Growth Factor Beta 1 Protein Coding Gene
gcaagtggacatcaacgggt

SEQ ID NO: 8 - DNA, 20 bp, Unknown
  CDS: Reverse Primer Transforming Growth Factor Beta 1 Protein Coding Gene
tccgtggagctgaagcaata

SEQ ID NO: 9 - DNA, 23 bp, Unknown
  CDS: Forward Primer Matrix Metalloproteinase 3 Protein Coding Gene
tggacaaaggatacaacagggac

SEQ ID NO: 10 - DNA, 20 bp, Unknown
  CDS: Reverse Primer Matrix Metalloproteinase 3 Protein Coding Gene
atcttgagacaggcggaacc

SEQ ID NO: 11 - DNA, 21 bp, Unknown
  CDS: Forward Primer Matrix Metalloproteinase 13 Protein Coding Gene
aacgccagacaaatgtgaccc

SEQ ID NO: 12 - DNA, 20 bp, Unknown
  CDS: Reverse Primer Matrix Metalloproteinase 13 Protein Coding Gene
tccgcatcaacctgctgagg

SEQ ID NO: 13 - DNA, 20 bp, Unknown
  CDS: Forward Primer ADAM Metallopeptidase Protein Coding Gene
gcaacgtcaaggctcctctt

SEQ ID NO: 14 - DNA, 25 bp, Unknown
  CDS: Reverse Primer ADAM Metallopeptidase Protein Coding Gene
ctccacaaatctactcagtgaagca

SEQ ID NO: 15 - DNA, 20 bp, Unknown
  CDS: Forward Primer Glyceraldehyde Protein Coding Gene
caaggctgagaacgggaagc

SEQ ID NO: 16 - DNA, 20 bp, Unknown
  CDS: Reverse Primer Glyceraldehyde Protein Coding Gene
agggggcagagatgatgacc

SEQ ID NO: 17 - DNA, 21 bp, Unknown
  CDS: Forward Primer Transforming Growth Factor Beta 1 Protein Coding Gene
cccctggaaagggctcaacac

SEQ ID NO: 18 - DNA, 25 bp, Unknown
  CDS: Reverse Primer Transforming Growth Factor Beta 1 Protein Coding Gene
tccaacccaggtccttcctaaagtc

SEQ ID NO: 19 - DNA, 18 bp, Unknown
  CDS: Forward Primer Ribosomal Protein L19 Protein Coding Gene
tgccggaagaacaccttg

SEQ ID NO: 20 - DNA, 19 bp, Unknown
  CDS: Reverse Primer Ribosomal Protein L19 Protein Coding Gene
gcaggatcctcatccttcg

SEQ ID NO: 21 - DNA, 20 bp, Unknown
  CDS: Forward Primer Beta actin Protein Coding Gene
ccaaccgtgaaaagatgacc

SEQ ID NO: 22 - DNA, 20 bp, Unknown
  CDS: Reverse Primer Beta actin Protein Coding Gene
accagaggcatacagggaca

(19) United States
(12) Patent Application Publication
(10) Pub. No.: US 2022/0000964 A1
(43) Pub. Date: Jan. 6, 2022

(54) Compositions and Methods for Regulating Homeostasis of Chondrocytes, Extracellular Matrix, Articular Cartilage, and Phenotype of Arthritis

(71) Applicant: Unigen, Inc., Tacoma, WA (US)

(72) Inventors: Mesfin Yimam, Tacoma, WA (US); Mei Hong, Tacoma, WA (US); Ping Jiao, Newcastle, WA (US); Teresa Horm, Renton, WA (US); Kelly Dawkins, Tacoma, WA (US); Alexandria O'Neal, Orting, WA (US); Qi Jia, Olympia, WA (US)

(73) Assignee: Unigen, Inc., Tacoma, WA (US)

(21) Appl. No.: 17/169,490

(22) Filed: Feb. 7, 2021

Related U.S. Application Data
(60) Provisional application No. 62/970,792, filed on Feb. 6, 2020.

(51) Int. Cl. A61K 36/9062; A61K 36/575; A61K 36/21; A61K 36/19; A61K 36/80; A61K 36/25; A61K 36/13; A61K 36/282; A61K 36/75; A61K 36/57; A61K 36/185; A61K 36/53; A61K 36/67; A61K 31/05; A61K 31/352; A61K 31/12; A61K 36/906; A61K 35/60; A61K 36/82; A61K 36/87; A61K 31/165; A61K 31/375; A61K 31/593; A61K 31/355; A61P 19/02

(52) U.S. Cl. CPC A61K 36/9062; A61P 19/02; A61K 36/21; A61K 36/19; A61K 36/80; A61K 36/25; A61K 36/13; A61K 36/282; A61K 36/75; A61K 36/57; A61K 36/185; A61K 36/53; A61K 36/67; A61K 31/05; A61K 31/352; A61K 31/12; A61K 36/906; A61K 35/60; A61K 36/82; A61K 36/87; A61K 31/165; A61K 31/375; A61K 31/593; A61K 31/355; A61K 36/575

(57) ABSTRACT

Medicinal plant extracts and their bioactives from Alpinia, Magnolia, Kochia and Piper/Pepper are disclosed herein in combination or alone in regulating homeostasis of chondrocytes, extracellular matrix, articular cartilage, and phenotype of arthritis that lead to enhanced anabolic functions of chondrocytes, increased renewal/rebuilding/regeneration of extracellular matrix and articular cartilage, and improved phenotype of osteoarthritis and rheumatoid arthritis.

This application is a United States Utility application that is based on and claims priority to U.S. Provisional Patent Application Ser. No. 62/970,792 filed on Feb. 6, 2020 and entitled “Compositions and Methods for Regulating Homeostasis of Chondrocytes, Extracellular Matrix, Articular Cartilage, and Phenotype of Arthritis”, which is commonly owned and incorporated in its entirety by reference.

### BACKGROUND

Osteoarthritis (OA) is a multifactorial disease that affects the entire joint structure and is characterized by cartilage destruction and loss, degeneration of soft tissues, localized bone hypertrophy including subchondral thickening and osteophyte formation, varying degrees of synovitis, and thickening of the joint capsule (Loeser, 2013).

Over the years, progress has been made to address the symptoms, especially pain pathways, but not key detrimental factors driving the development and progression of OA (Wenham and Conaghan, 2013). The compilation of evidence from augmented sources suggests that osteoarthritis is no longer considered as a “wear and tear” degenerative disease anticipated to happen as a consequence of aging or no longer considered to be a “noninflammatory” form of arthritis. Using a modern imaging technology such as MRI, for example, synovial membrane inflammation has been shown to be correlated with high prevalence to the severity and progression of OA and was believed to be the primary cause of pain (Pickering et al., 2005; Roemer et al., 2011). Immunological changes such as infiltration of B cells in the synovium and activation of T cells have also been reported to take part in not only the pathogenesis of rheumatoid arthritis (RA) but also the pathogenesis of OA (Qin et al., 2007; Sakkas and Platsoucas, 2007).

Substantial reports have shown the elusive nature to specifically single out an etiology for OA which indicates the intertwined existence of multiple factors involving mechanical and molecular events in the initiation and progression of the disease. It is cumbersome to pinpoint exactly when and where the disease originated as patients often seek help after significant structural damage that has already occurred; nevertheless, it believed that the strong correlation among synovitis, cartilage, and meniscus degradation has been described as part of a vicious circle perpetuating OA (Roemer et al., 2013).

Although cartilage destruction is the main event in defining osteoarthritis, the degradation of type II collagen is the fundamental incident that is believed to be the irreversible progression of osteoarthritis disease in association with inflammation. Progressive degradation of articular cartilage is the hallmark of OA. Articular cartilage is an avascular, non-innervated tissue composed of a dense extracellular matrix (ECM) with a sparse distribution of highly specialized cells called chondrocytes. Chondrocytes originate from mesenchymal stem cells and constitute about 2% of the total volume of articular cartilage (Alford and Cole, 2005). Chondrocytes are metabolically active cells that play a pivotal role in the development, maintenance, and repair of the ECM which is mainly composed of type II collagen and aggrecan. Collagen is the most abundant structural macromolecule in ECM where type II collagen represents 90%-95% of the collagen in the tissue and forms fibers intertwined with proteoglycan aggregates. Proteoglycans are heavily glycosylated protein monomers representing the second-largest group of macromolecules in the ECM and account for up to 10%-15%. Proteoglycans consist of a protein core with one or more linear glycosaminoglycan (GAG) chain covalently attached. These structures provide the visco-elasticity property and resistance to compression forces to the articular cartilage.

Homeostasis and integrity of extracellular matrix (ECM) is fundamental for proper function of articular cartilage to maintain a healthy joint. Several mechanical, biochemical, and micro-environmental factors can regulate the metabolic activity of chondrocytes within the ECM of articular cartilage. Accordingly, different anabolic signals perceived by the chondrocyte will lead to the production, organization, and maintenance of the integrity of cartilage ECM. Abnormal and catabolic signals due to increased production of matrix metalloproteinases (MMPs) and proteoglycanases by chondrocytes in the affected structures of the joint can shift the homeostasis of ECM to the catabolic side and lead to the degradation of the ECM. This is the main characteristic of both osteo- and rheumatoid arthritis. Pro-inflammatory cytokines, such as TNF-α, IL-1β, and IL-6 are known to play important roles in cartilage matrix degradation in articular cartilage through a cascade of catabolic events that lead to stimulation of aggrecanase and matrix metalloproteinase (MMP) secretion (Kapoor et al., 2011). Besides the disruption of cartilage matrix homeostasis and integrity, these collective catabolic mediators decrease the response and sensitivity of chondrocytes to the surrounding anabolic signals, further shifting the balance more towards catabolic cartilage degradation than anabolic rebuilding and renewal of ECM and cartilage. As a result, natural compositions with the capacity of reversing the direction from catabolic to anabolic processes could act as disease-modifying agents and have beneficial effects, such as modifying, slowing down, or reversing the progression of arthritis.

Present-day management of OA is inadequate due to the lack of primary therapies proven to be effective in hindering disease cause and progression. The current pharmaceutical approach, which focuses mainly on curtailing the symptoms of disease, mainly, associated pain, will only mask the actual etiology but not balance the catabolic-anabolic homeostasis, leading to irreversible damage to the cartilage integrity and joint structure. While intra-articular injection of corticosteroids, hyaluronic acid, and oral or topical nonsteroidal anti-inflammatory drugs (NTHEs) have most frequently been used to relieve pain and stiffness in OA patients, glucosamine and chondroitin have also shown delayed, but measurable, outcome on pain and improved function in more severe stages of OA. In fact, previously, glucosamine sulfate and chondroitin sulfate were recommended by the Osteoarthritis Research Society International (OARSI) as possible structural modifying agents in hip and knee OA (Jordan et al., 2003; Zhang et al., 2007). However, the recently published OARSI guidelines downgraded these agents to “uncertain” as a symptom reliever or “not appropriate” as a disease-modifying agent when used for all OA patients. Similarly, oral, and transdermal opioid painkillers were graded as “uncertain” for managing OA (Zhang et al., 2008, 2010). On the other hand, topical NTHEs are recommended as appropriate for all patients with knee-only OA and were found to be safer and better tolerated compared to oral NTHEs (McAlindon et al., 2014). These periodic changes in recommendations of use by the expert panel clearly define the uncertainty of current nonpharmacological and pharmacological modalities of therapy for OA management. Intensifying the complicated situation, many distressed patients compromise their safety by inclining more towards substandard and unregulated product sources, hoping to lessen the catastrophic outcome of the disease and to improve their quality of life. As a result, there still is an unmet need for evidence-based safe and efficacious alternatives from natural sources.

Rheumatoid arthritis (RA) is a chronic, inflammatory, autoimmune disease that primarily affects the joints (Smolen et al., 2018). Although RA is a systemic disease and a variety of immunological events occur outside the joint at mucosal surfaces and primary lymphoid tissues, the synovium is a central player. The disease is characterized by infiltration of the synovial membrane of joints with cellular and humoral immunity cells such as T cells, B cells, and monocytes. This process is preceded by neovascularization (activation of endothelial cells leading to growth of new blood vessels) which is considered as a hallmark of RA synovitis. Expansion of synovial fibroblast-like and macrophage-like cells in the synovial membrane leads to a hyperplastic synovial lining layer. This expanded synovial membrane, often termed “pannus,” invades the periarticular bone at the cartilage-bone junction and leads to bony erosions and cartilage degradation.

In the pathogenesis of RA, cytokine networks integrate pro-inflammatory and tissue-damaging cellular activities in synovitis. Proinflammatory cytokines, primarily TNF-α, and IL-6, are known to induce molecules such as receptor activator of nuclear factor KB ligand (RANKL), prostaglandins (PGE2), matrix metalloproteinases (MMP-13, MMP-3, MMP-9, MMP-1) and aggrecanases in RA. These factors mediate the signs and symptoms of RA. TNF-α, and IL-6 also stimulate generation of osteoclasts within the synovial membrane and promote bone damage. These molecular and cellular events result in the clinical disease expression manifested as pain, swelling (typically accompanied by morning stiffness and tenderness), deformity, and degradation of cartilage and bone. Damage to cartilage and bone due to synovial invasion into adjacent articular structures is one of the cardinal signs of RA (Smolen et al., 2018).

Like OA, the goals of treatment for RA are to reduce joint inflammation and pain, maximize joint function, and prevent joint destruction and deformity. Through the years, better understanding of the pathogenesis of RA (through recognition of key cells and cytokines) has led to dramatic improvements and the development of targeted disease-modifying antirheumatic drugs. In particular, rheumatologists have learned how to use the immunosuppressant methotrexate optimally, and this drug has become the therapeutic anchor for managing RA (Visser and van der Heijde, 2009). Well aligned with this understanding, besides histological findings in improvements of joint structure maintenance and protection of the subchondral bone, compositions disclosed in this disclosure produced comparable outcomes to methotrexate in symptomatic relief, and reduction of key inflammatory cytokines (TNF-α and IL6) and matrix degrading enzymes (MMP-13 and MMP-3) when tested in collagen-induced arthritis (CIA). This model is most frequently used as a disease model for testing efficacy of pharmaceutical and nutraceuticals in RA and/or the pathogenesis of RA (Cho et al., 2007). These findings suggest the natural compositions disclosed in the current application are suitable for the management of RA in addition to OA.

Herein, we document multiple natural extracts and their combined compositions that statistically and significantly reduced catabolic biomarkers for cartilage turnover—such as uCTX-II (primary marker for cartilage degradation) by down regulating catabolic cytokines (such as TNF-α, IL-1β, and IL-6) and extracellular matrix degrading enzymes (MMP3, 9, and 13) in animals treated with those compositions. These findings were also substantiated by modulating homeostasis of chondrocytes where the gene expression of catabolic pathways was significantly down regulated for matrix degrading enzymes (metalloproteinase and aggrecanase) such as MMP13, MMP3 and ADAMTS4 after oral treatment. These phenomena are key indicators of the current invented composition's activities in minimizing the catabolic processes of the phenotype of arthritis.

To date, there is no regulatory-approved disease-modifying drug for OA that could be applicable for regeneration of cartilage. In perspective, dietary supplements with multiple known mechanisms of actions could assist in the cartilage repairing process. In the current disclosure, the proprietary compositions consisting of, but not limited to, individual Alpinia, Pepper, Magnolia and Kochia extracts, and/or at various combinations of 2 to 3 of those extracts with examples of, but not limited to Alpinia:Pepper (AP) and Alpinia:Magnolia:Kochia (AMK), resulted in unexpected faster and improved cartilage repairing activity with synergy as reflected in the animal weight bearing data and histopathological observation of the cartilage repairing parameters in diseased animal models. The levels of cartilage synthesis markers, such as type IIA procollagen amino terminal propeptide (PIIANP) and the growth factor TGF-β1, were found significantly higher in rats treated with individual extracts of Alpinia, Pepper, Magnolia and Kochia and also by those compositions of AMK and AP when compared to vehicle-treated disease models. These compositions have also shown significant cartilage protection activity in the collagen-induced rat arthritis model and anti-pain and anti-inflammatory activity in the carrageenan-induced rat paw edema model. The merits of combining these extracts to yield, but not limited to, AP or AMK composition were also evaluated using the Colby's equation (Colby, 1967) and unexpected synergy was found for combined compositions. These wide array of activities demonstrated by individual Alpinia, Pepper, Magnolia and Kochia extracts and/or at various combinations of 2 to 3 of those extracts with examples, but not limited to, AP and AMK, could be attributed to the diverse nature of actives present in the compositions. The compiled data dictate that individual Alpinia, Pepper, Magnolia and Kochia extracts and/or at various combinations of 2 to 3 of those extracts with examples, but not limited to, AP and AMK compositions provide symptom relief, anti-catabolic joint cartilage protection and anabolic joint cartilage repair—triple function, which could be a holistic approach as a disease-modifying agent for osteoarthritis.

In the present disclosure, data depicted in this patent details the novelty of the individual Alpinia, Pepper, Magnolia and Kochia extracts and/or at various combinations of 2 to 3 of those extracts with examples, but not limited to Alpinia:Pepper (AP) and Alpinia:Magnolia:Kochia (AMK) compositions to address the unmet need for regulating homeostasis of chondrocytes, the extracellular matrix, articular cartilage, and the phenotype of arthritis. Administered at the exampled combination ratios, individual Alpinia, Pepper, Magnolia and Kochia extracts, and/or at various combinations of 2 to 3 of those extracts with examples, but not limited to, AP and AMK, reversed the course of OA towards normal or anabolic homeostasis balance by inducing cartilage synthesis and by inhibiting ECM degradation. We believe that natural compositions like individual Alpinia, Pepper, Magnolia and Kochia extracts and/or at various combinations of 2 to 3 of those extracts with examples, but not limited to AP and AMK, compositions possess unique capacities in stimulating anabolic gene expression and inhibiting catabolic activities which establish those compositions as preferred choices for OA/RA disease-modifying agents from natural sources.

Enteral and parenteral routs of drug administration are among the commonly used methods of drug delivery for patients suffering from musculoskeletal pain. However, the commonly prescribed or over the counter anti-pain medications such as selective and non-selective nonsteroidal anti-inflammatory drugs are known to cause gastrointestinal, cardiovascular, and renal side effects (Harirforoosh et al., 2013). Older patients who actually often experience chronic pain are at greater risk of side effects from these routes of intervention (Stanos and Galluzzi). These adverse events could be averted by employing NTHEs by a topical application route. Applying anti-pain products directly to the affected area, such as in cases of muscular strain, sprains, osteoarthritis, rheumatoid arthritis and other spectrums of musculoskeletal conditions, could result in a high concentration of active compounds at the intended target areas, yielding fast and robust pain relief while minimizing systemic exposure (Rodriguez-Merchan, 2018; Argoff, 2013). Nevertheless, there still is an unmet need for topically applicable medications or alternatives with improved efficacy for musculoskeletal disorders. We believe that natural products with diverse chemical entities and mechanisms of action could help to bridge the gap for topical alternatives. In the work on contemplated embodiments, we screened and evaluated our plant library for topical analgesics and hypothesized their potentially enhanced pain relief activity as a result of standardized formulations and improved skin penetration. Part of the discovery processes of novel, topically effective anti-pain formulations have been documented in the conceptualization of contemplated embodiments disclosed herein.

Depending on the initial stimuli, pain could be nociceptive, inflammatory, or neuropathic. It has been hypothesized that these medicinal plants could cause suppression in pain sensitivity by directly interfering with the peripheral primary afferent sensory neurons at the receptor level or indirectly by acting through the many pathways of pain transduction, transmission, modulation, and perception. Bradykinin and prostaglandins are among the classic inflammation mediators known to cause pain sensitivity in inflammation.

### SUMMARY OF THE SUBJECT MATTER

Medicinal plant extracts and their bioactives from Alpinia, Magnolia, Kochia and Piper/Pepper are disclosed herein in combination or alone in regulating homeostasis of chondrocytes, extracellular matrix, articular cartilage, and phenotype of arthritis that lead to enhanced anabolic functions of chondrocytes, increased renewal/rebuilding/regeneration of extracellular matrix and articular cartilage, and improved phenotype of osteoarthritis and rheumatoid arthritis. The shifting of balance at a cellular and tissue level not only preserved/protected/improved/renewed structural integrity of extracellular matrix and articular cartilage, but also protected/improved/enhanced joint/bone structure and joint function, observed as reduced joint inflammation, joint pain, joint stiffness, decreased cartilage degradation, improved mobility, range of motion, flexibility, joint physical function, or any combination thereof.

The anti-catabolic and pro-anabolic activities of individual extracts of Alpinia, Pepper, Magnolia and Kochia and/or at various combinations of 2 to 3 of those extracts with examples, but not limited to Alpinia:Piper Pepper (AP) and Alpinia:Magnolia:Kochia (AMK) were demonstrated at molecular and cellular levels, including gene expression, protein expression, and protein function reduction; at tissue levels with biomarker-guided tissue protection; at diseased animal models, with not only symptom relief, but by anabolic and catabolic biomarker changes and improvements of histopathological images and scores.

The methods of use of the disclosed individual extracts of Alpinia, Pepper, Magnolia and Kochia and/or at various combinations of 2 to 3 of those extracts with examples, but not limited to Alpinia:Piper Pepper (AP) and Alpinia:Magnolia:Kochia (AMK) include, but are not limited to, maintaining cartilage homeostasis, extracellular matrix integrity, and joint cartilage; minimizing cartilage degradation, protecting joint space from narrowing, and promoting healthy joints by protecting cartilage integrity; balancing anabolic and catabolic processes, diminishing the actions of enzymes and proinflammatory cytokines that affect joint health, improving joint movement and/or function, alleviating joint pain, alleviating joint stiffness, improving joint range of motion and/or flexibility, promoting mobility, managing and/or treating osteoarthritis and/or rheumatoid arthritis, preventing osteoarthritis and/or rheumatoid arthritis, or reversing the progression of osteoarthritis and/or rheumatoid arthritis or the like.

Specifically, a composition for joint health is disclosed that comprises a combination of an Alpinia extract enriched for one or more phenylpropanoids; a Magnolia extract enriched for one or more bisphenolic lignans; and a Kochia extract enriched for one or more triterpenoid saponins.

In additional embodiments, a composition for joint health is disclosed that comprises a combination of an Alpinia extract enriched for one or more phenylpropanoids; and a Piper extract enriched for one or more alkaloids.

In yet additional embodiments, a composition for joint health is disclosed that comprises an Alpinia extract enriched for one or more phenylpropanoids.

### BRIEF DESCRIPTION OF THE FIGURES

FIG. 1 shows individual extracts of Alpinia, Piper/Pepper, Magnolia and Kochia and/or at various combinations of 2 to 3 of those extracts with examples, but not limited to Alpinia:Piper/Pepper (AP) and Alpinia:Magnolia:Kochia (AMK), reverses OA progression by inducing cartilage homeostasis.

FIG. 2 shows a HPLC chromatogram of Alpinia ethanol extract at 254 nm.

FIG. 3 shows images of a drill site of OCD rats after 6 weeks of treatment showing significant differences in healing progress from different oral treatment groups.

FIG. 4 shows Safranin O stain of the subchondral bone of OCD rats at the drill site. The black circle indicates the drill site for representative animal histopathology slides.

FIG. 5 shows histopathology images (HE a-d and Safranin O e-f) from ankle joint of CIA induced rats treated with AMK and MTX. A and e—normal control, b and f—CIA+vehicle, c and g—CIA+MTX, d and h—CIA+AMK.

FIG. 6 shows HE and Safranin O staining histology for CIA rats treated with AP (HE stains (40×): a=normal control+Vehicle, b=CIA+Vehicle, c=CIA+Methotrexate, d=CIA+AP, Safranin O stain (40×): e=normal control+vehicle, f=CIA+Vehicle, g=CIA+Methotrexate, h=CIA+AP, C=cartilage, SB=subchondral bone, I=inflammation)

### DETAILED DESCRIPTION

Osteoarthritis (OA) is a multifactorial disease primarily noted by cartilage degradation that causes significant morbidity, joint pain, stiffness, and limited mobility. Present-day management of OA is inadequate due to the lack of principal therapies proven to be effective in hindering disease progression wherein a symptomatic therapy-focused approach, such as the use of nonsteroidal anti-inflammatory drugs, masks the actual etiology leading to irreversible cartilage depletion and joint structural damage. Here we present the discovery of novel natural extracts and compositions designated as examples of, but not limited to, Alpinia, Piper/Pepper, Magnolia and Kochia extracts and at various combinations which resulted in unexpected faster and improved cartilage renewal and repairing activity with synergy. These activities derived from individual Alpinia, Piper/Pepper, Magnolia and Kochia extracts and/or at various combinations of 2 to 3 of those extracts with examples, but not limited to Alpinia:Piper/Pepper (AP) and Alpinia:Magnolia:Kochia (AMK) are demonstrated in the examples 2, 9, 13, 17, and 18 in the current subject matter and reflected by inhibition of the release of glycosaminoglycan (GAG) from rabbit cartilage explants stimulated by a catabolic cytokine, interleukin-1; in the animal weight bearing data from the osteochondral model (OCD) (examples 25 to 31) and in the histopathological observation of cartilage protection, repairing and renewal parameters in collagen-induced arthritis (CIA) (examples 40 to 58), monoiodoacetate-induced arthritis (MIA) (examples 59 to 63) and OCD (examples 25 to 31) diseased animal models for regulating homeostasis of chondrocytes, extracellular matrix, articular cartilage, and phenotype of arthritis.

Specifically, a composition for joint health is disclosed that comprises a combination of an Alpinia extract enriched for one or more phenylpropanoids; a Magnolia extract enriched for one or more bisphenolic lignans; and a Kochia extract enriched for one or more triterpenoid saponins. Contemplated compositions are developed such that the Alpinia extract, or Magnolia extract or Kochia extract in the composition are in a range of 1%-98% by weight of each extract with the optimized weight ratio of Alpinia:Magnolia:Kochia (AMK) at 2:4:3 (22.2%:44.4%:33.3%) or 4:3:3 (40%:30%:30%) or 5:4:4 (38.4%:30.8%:30.8%).

In additional embodiments, a composition for joint health is disclosed that comprises a combination of an Alpinia extract enriched for one or more phenylpropanoids; and a Piper extract enriched for one or more alkaloids.

In yet additional embodiments, a composition for joint health is disclosed that comprises an Alpinia extract enriched for one or more phenylpropanoids.

While chondrocytes respond to a variety of stimuli, including growth factors, they have limited potential for replication, a factor that contributes to the limited intrinsic healing capacity of cartilage in response to injury. Chondrocytes regulate cartilage homeostasis by maintaining a delicate balance between anabolic (regenerative) and catabolic (degradative) activities. These cells represent only 1-2% of the total matrix volume. They are avascular and unable to divide in adulthood, causing a very limited ability for cartilage self-repair and low turnover rate. They usually acquire their nutrition and oxygen primarily through diffusion from the synovial fluid and subchondral bone. Chondrocytes maintain the homeostasis of articular cartilage matrix by modulating the balance between the synthesis and degradation of various articular components. This process is controlled by the relative levels of cytokines and growth factors in the surrounding tissues, such as cartilage and/or synovial fluid and/or synovial membrane. Chondrocytes can maintain the integrity of extracellular matrix (ECM) by synthesis of macromolecules such as type II collagen and aggrecans and they can also produce proteins involved in the degradation of ECM such as MMPs and aggrecanases. As chondrocytes are very responsive and sensitive to changes to their micro-environment, natural extracts and compositions that stimulate the chondrocytes directly or indirectly to produce matrix-forming components and inhibit the secretion of proinflammatory cytokines and matrix degrading enzymes could change the homeostasis of the ECM and the phenotype of arthritis. In the current subject matter, we have documented data in the examples substantiating the cartilage rebuilding and renewal capacity of individual Alpinia, Pepper, Magnolia and Kochia extracts and/or at various combinations of 2 to 3 of those extracts with examples of, but not limited to, compositions Alpinia:Piper/Pepper (AP) and Alpinia:Magnolia:Kochia (AMK) in OCD model in addition to their cartilage protection and symptom relief activities in the CIA, MIA and carrageenan models outweighing the degradation process to maintain homeostasis.

During chondrogenesis, mesenchymal stem cell (MSC) condensation and subsequent chondrocyte differentiation are the initial steps in cartilage formation. These processes are driven by several growth and transcription factors at different stages of cartilage development. Among these factors, SOX9, a key transcription factor for chondrogenesis, is involved in the condensation phase of MSCs, stimulating the expression of cartilage-specific markers and inhibiting terminal differentiation of chondrocytes. Similarly, the TGF-β family of genes is widely expressed in chondrocytes and is a constituent class of growth factors involved in the process of chondrogenesis. Of all the factors expressed during the early stages of chondrogenesis, TGF-β1 is one of the most important factors that induces the differentiation of MSCs into chondrocytes. This factor also stimulates the proliferation of chondrocytes, increases the production of ECM, and it inhibits endochondral ossification. In this anabolic phase of cartilage development (stimulated by SOX9 and TGF-β1), mature chondrocytes will produce cartilage matrix rich in proteoglycan and type II collagen fibers encoded by ACAN and COL2A1 genes, respectively. As a result, external factors that upregulate the expression of the transcription or growth factors help induce the anabolic process of cartilage development to maintain the surplus of ECM. In fact, in our discovery process of natural compositions derived from individual Alpinia, Pepper, Magnolia and Kochia extracts and/or at various combinations of 2 to 3 of those extracts with examples, but not limited to Alpinia:Piper/Pepper (AP) and Alpinia:Magnolia:Kochia (AMK) found upregulation of SOX 9, TGF-β1, ACAN and COL2A1 genes in ex vivo, and in vitro models in IL-1β-stimulated human chondrocytes demonstrated in current subject matter examples 21, 22, 23 and 24. These findings of regulating homeostasis of chondrocytes, extracellular matrix, articular cartilage, and phenotype of arthritis were later reinforced by in vivo results from CIA, MIA and OCD models demonstrated in the current subject matter. The levels of cartilage collagen synthesis markers, such as type IIA procollagen amino terminal propeptide (PIIANP) (Examples 40, 48, 56 and 58) and the growth factor TGF-β1 (example 31), were found significantly higher in rats treated with individual extracts of Alpinia, Pepper, Magnolia and Kochia and/or at various combinations of 2 to 3 of those extracts with examples, but not limited to Alpinia:Piper/Pepper (AP) and Alpinia:Magnolia:Kochia (AMK) when compared to vehicle-treated disease animals. These phenomena such as upregulation of anabolic gene markers which are directly involved in cartilage synthesis and renewal address, in part, the activities of the disclosed compositions.

To the best of our knowledge, this is the first time that the disclosed medicinal plants have been evaluated in the given ratio for their ability to retain and rebuild cartilage in the osteochondral defect (OCD) model, resulting in favorable outcomes. This model has a direct implication in assessing interventions for their cartilage renewal and rebuilding function. Cartilage synthesis and hence disease-modifying activity of individual extracts of Alpinia, Pepper, Magnolia and Kochia and/or at various combinations of 2 to 3 of those extracts with examples, but not limited to, Alpinia:Piper/Pepper (AP) and Alpinia:Magnolia:Kochia (AMK), were demonstrated using the osteochondral defect (OCD) model in rats as illustrated in the examples 25, 26, 27, 28, 29, 30 and 31. The model utilizes stimulation of the bone marrow in the repairing process by taking advantage of the body's own healing potential. This technique enhances the chondral resurfacing by providing a suitable environment for new tissue formation. At the time of model induction, the exposed weight-bearing surface of the femur subchondral bone plate was drilled with a precision drill bit until fat droplets and blood came out of the microfractured hole in the knee. This provided an optimal environment for the body's own mesenchymal stem cells from the bone marrow to differentiate into appropriate articular cartilage-like cells that in turn produced the extracellular matrix which eventually matured into stable repaired tissue. The cartilage repair activity of individual extracts of Alpinia, Pepper, Magnolia and Kochia and/or at various combinations of 2 to 3 of those extracts with examples, but not limited to Alpinia:Piper/Pepper (AP) and Alpinia:Magnolia:Kochia (AMK) were evaluated using this model administered orally at different dosages and time periods, such as 200 mg/kg/day for 8 weeks. As a means of assessing the repair progress, the distribution of weight-bearing between the right and left legs of rats were measured using the incapacitance tester. At necropsy, serum for biomarkers and the left knees for histopathology were collected. Images of the left knee focused on the drilling site were taken for all the animals before fixing them with formalin. Fixed tissues were processed and analyzed by an independent and certified pathologist.

OCD animals exhibited limping on the affected legs which showed progressive improvement through the course of the study for all the groups. These changes in the open field observation of the use of their affected legs were also reflected in the incapacitance measurements. There was gradual improvement in the weight-bearing measurements that was significantly improved for rats treated with Alpinia:Piper/Pepper (AP) and Alpinia:Magnolia:Kochia (AMK) compositions. After 6 weeks of daily oral treatment, rats treated with AMK and AP compositions showed 59.9% and 51.5% improvement, respectively, in the use of their affected legs to carry their body weight. This was an indication of reduced pain in the surgically drilled knee. These values seemed to match what was observed in the pictures taken at necropsy from the AMK and AP groups relative to the vehicle-treated OCD animals. These findings were also substantiated by the histopathology data, which were analyzed using the Sellers method of analysis described in the body of the patent for cartilage repair, which showed 40.4% and 40.5% accelerated healing in animals treated with AMK and AP compared to the vehicle-treated disease model. These improvements were statistically significant for AMK- and AP-treated OCD rats compared to the vehicle treated group. These findings reflect the cartilage synthesis (anabolic) and stimulation activity of AMK and AP in vivo complementing the upregulated in vitro anabolic gene markers justifying their cartilage rebuilding and renewal activities.

The cartilage protection activities of individual extracts of Alpinia, Pepper, Magnolia and Kochia and/or at various combinations of 2 to 3 of those extracts with examples, but not limited to Alpinia:Piper/Pepper (AP) and Alpinia:Magnolia:Kochia (AMK), were also demonstrated in additional animal models. In the CIA and MIA induced arthritis models demonstrated in examples 40, 48 56 and 58, individual extracts of Alpinia, Pepper, Magnolia and Kochia and/or at various combinations of 2 to 3 of those extracts with examples, but not limited to, AP and AMK produced statistically significant reductions in urinary CTX-II, a primary biomarker for cartilage degradation and statistically significant increases in a cartilage synthesis biomarker, PIIANP. Those individual extracts of Alpinia, Pepper, Magnolia and Kochia and/or at various combinations of 2 to 3 of those extracts with examples, but not limited to, AP and AMK also showed a statistically significant decrease in serum catabolic biomarkers such as IL-1β, TNF-α, and IL-6 levels as well as different MMP enzymes which are considered as the primary catabolic pathways associated with inflammatory cytokines and matrix degrading enzymes. Data from these models suggest the anti-catabolic activity of these individual extracts and combined compositions.

Substantiating the in vivo observations, individual extracts of Alpinia, Pepper, Magnolia and Kochia and/or at various combinations of 2 to 3 of those extracts with examples, but not limited to Alpinia:Piper/Pepper (AP) and Alpinia:Magnolia:Kochia (AMK), upregulated expression of articular cartilage extracellular matrix anabolic biomarkers such as COL2A1 (the gene encoding type-II collagen) and ACAN (the gene encoding the cartilage-specific proteoglycan core protein) and downregulated expression of matrix catabolic homeostasis biomarkers such as MMP13, MMP3 and ADAMTS4 when human chondrocytes were incubated with a catabolic cytokine, IL-1. Articular cartilage matrix synthesis transcription factor, SOX9, and growth factor TGF-β1 (example 23 and 24) were also found up regulated in IL-1-stimulated human chondrocytes treated with those individual extracts of Alpinia, Pepper, Magnolia and Kochia and also those examples, but not limited to, AP and AMK compositions. These findings show that individual extracts of Alpinia, Pepper, Magnolia, and Kochia and also compositions of these plant extracts, not limited to AMK and AP, promote cartilage regeneration by increasing the levels of master regulators of cartilage synthesis, TGF-β1 and SOX9, leading to the increase of cartilage components, ACAN, COL2A1, and PIIANP. Conversely, the extracts decreased the expression and activity of MMP13, MMP3, ADAMTS4, and MMP9, enzymes that are responsible for the majority of direct cartilage breakdown. The net result of these activities is maintenance of remaining cartilage and initiation of cartilage synthesis to restore integrity to the architecture of the joint.

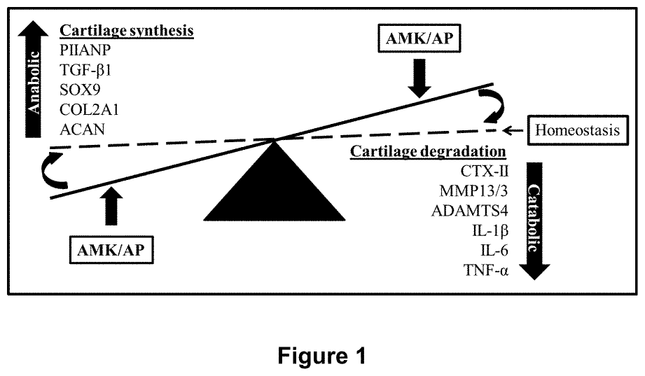

Although the initial etiology of OA/RA is under debate, homeostasis disturbances as a result of cartilage synthesis and degradation imbalance play a key role in the initiation and progression of osteoarthritis and also rheumatoid arthritis. Data presented in this disclosure showed the effect of these individual extracts of Alpinia, Pepper, Magnolia and Kochia and/or at various combinations of 2 to 3 of those extracts with examples, but not limited to, Alpinia:Piper/Pepper (AP) and Alpinia:Magnolia:Kochia (AMK), in reversing the direction of arthritis progression towards normal and/or anabolic homeostasis by inducing cartilage synthesis (and hence, anabolic effect) and inhibiting the catabolic process of degradation and breakdown. FIG. 1 shows individual extracts of Alpinia, Piper/Pepper, Magnolia and Kochia and/or at various combinations of 2 to 3 of those extracts with examples, but not limited to Alpinia:Piper/Pepper (AP) and Alpinia:Magnolia:Kochia (AMK), reverses OA progression by inducing cartilage homeostasis.

We believe that the multifactorial complexity of pain may suggest the need for an intervention strategy that involves the combination of two or more active extracts together to elicit multiple approaches: enhanced pain relief, alleviation of cartilage breakdown, and initiation of cartilage synthesis. The in vivo studies we conducted using Alpinia, Pepper, Magnolia, and Kochia, as well as compositions not limited to Alpinia:Piper/Pepper (AP) and Alpinia:Magnolia:Kochia (AMK), demonstrated enhanced pain relief, as well as histological alleviation of cartilage breakdown. The in vitro and ex vivo studies we demonstrated in the current subject matter using the plant extracts individually and in combination showed a reduction in cartilage degradation, as well as increased cartilage synthesis for several individual extracts as well as the combinations. Notably, the individual extracts did not all display each of these activities, but compositions of the extracts augmented their individual activities, achieving unexpected efficacy with synergy in these intervention activities.

In a topical treatment paradigm, there is a greater advantage in the use of penetration enhancers to increase the degree of absorption and to facilitate transdermal permeation overcoming the stratum corneum barrier. With respect to this, we considered the use of aloe in our formulations and added aloe at 2% during the preparation for some of the extracts as indicated in the examples (Fox et al., 2015).

Known topically active NTHE drugs had been formulated at 5% Ibuprofen or 1% Diclofenac, as NTHE controls in the current evaluation. Two over the counter (OTC) actives were also obtained to make 0.5% Capsaicin or 5% Menthol as OTC positive control. Commercial OTC pain relief products, such as BENGAY®, have also been utilized as controls.

An in vivo hot plate test was utilized as the testing model to evaluate the topical pain relief function of the selected natural leads against known positive NTHEs and OTC controls. A small amount of DMSO was utilized to dissolve botanical extracts or compounds at 5% concentration. DMSO sample solutions were mixed with equal volumes of Aloe vera gel (2-4% Aloe leaf gel powder in DI water), which was applied topically to rat paws before the hot plate experiment (example 64).

Pain is a multifactorial phenomenon triggered by multiple mechanisms. Application of these test materials could be involved in, but not limited to, the initial activation and subsequent desensitization of peripheral nerve fibers, competitive inhibition or activation of transient receptor potentials such as TRPV1 and/or TRPA1, modulations of cannabinoid receptors (CB1 and CB2 receptors), antagonization and/or blocking of TRPV1 and TRPA1, an initial increase in release of substance-P followed by a depletion, inhibition of bradykinin activity, and inhibition of peripheral synthesis of inflammatory mediators, such as prostaglandin, bradykinin and cytokines. Hence, given the diverse nature of bioactives present in the tested medicinal plant materials, the current topical anti-pain data depicted in this subject matter, in association with the carrageenan, the MIA, the CIA and OCD model data, could expand the use of individual extracts of Alpinia, Piper/Pepper, Magnolia, and Kochia and also compositions of these plant extracts, not limited to Alpinia:Piper/Pepper (AP) and Alpinia:Magnolia:Kochia (AMK), by combining these plant materials in a specific ratio for enhanced pain relief activity.

In the above and following descriptions, certain specific details are set forth in order to provide a thorough understanding of various embodiments of this disclosure. However, one skilled in the art will understand that the disclosure may be practiced without these details.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “consisting essentially of” mean ±20% of the indicated range, value, or structure, unless otherwise indicated. It should be understood that the terms “a” and “an” as used herein refer to “one or more” of the enumerated components. The use of the alternative (e.g., “and/or”) should be understood to mean either one, both, or any combination thereof of the alternatives. Unless the context requires otherwise, throughout the present specification and claims, the word “comprise” and variations thereof, such as, “comprises” and “comprising,” as well as synonymous terms like “include” and “have” and variants thereof, are to be construed in an open, inclusive sense; that is, as “including, but not limited to.”

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment.

The term “prodrug” is also meant to include any covalently bonded carriers, which release the active compound of this disclosure in vivo when such prodrug is administered to a mammalian subject. Prodrugs of a compound of this disclosure may be prepared by modifying functional groups present in the compound of this disclosure in such a way that the modifications are cleaved, either in routine manipulation or in vivo, to the parent compound of this disclosure. Prodrugs include compounds of this disclosure wherein a hydroxy, amino or mercapto group is bonded to any group that, when the prodrug of the compound of this disclosure is administered to a mammalian subject, cleaves to form a free hydroxy, free amino or free mercapto group, respectively. Examples of prodrugs include acetate, formate and benzoate derivatives of alcohol or amide derivatives of amine functional groups in the compounds of this disclosure and the like.

The term “joint” health are meant to indicate improving the health of one or multiple “joints” of hand, elbow joints, wrist joints, axillary articulations, sternoclavicular joints, vertebral articulations, temporomandibular joints, sacroiliac joints, hip joints, knee joints and articulation of foot.

“Stable compound” and “stable structure” are meant to indicate a compound that is sufficiently robust to survive isolation to a useful degree of purity from a reaction mixture, and formulation into an efficacious therapeutic agent.

“Biomarker(s)” or “marker(s)” component(s) or compound(s) are meant to indicate one or multiple indigenous chemical component(s) or compound(s) in the disclosed plant(s), plant extract(s), or combined composition(s) with 2-3 plant extracts that are utilized for controlling the quality, consistence, integrity, stability, and/or biological functions of the invented composition(s).

“Mammal” includes humans and both domestic animals, such as companion animals, laboratory animals or household pets (e.g., cats, dogs, swine, cattle, sheep, goats, horses, rabbits), and non-domestic animals, such as wildlife or the like.

“Optional” or “optionally” means that the subsequently described element, component, event or circumstances may or may not occur, and that the description includes instances where the element, component, event or circumstance occur and instances in which they do not. For example, “optionally substituted aryl” means that the aryl radical may or may not be substituted and that the description includes both substituted aryl radicals and aryl radicals having no substitution.

“Pharmaceutically or nutraceutically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically or nutraceutically acceptable salt” includes both acid and base addition salts. “Pharmaceutically or nutraceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically or nutraceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include the sodium, potassium, lithium, ammonium, calcium, magnesium, iron, zinc, copper, manganese, aluminum salts and the like. In certain embodiments, the inorganic salts are ammonium, sodium, potassium, calcium, or magnesium salts. Salts derived from organic bases include salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly useful organic bases are isopropylamine, diethylamine, ethanolamine, trimethylamine, dicyclohexylamine, choline and caffeine.

Often crystallizations produce a solvate of the compound of this disclosure. As used herein, the term “solvate” refers to an aggregate that comprises one or more molecules of a compound of this disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. The compound of this disclosure may be true solvates, while in other cases, the compound of this disclosure may merely retain adventitious water or be a mixture of water plus some adventitious solvent.

A “pharmaceutical composition” or “nutraceutical composition” refers to a formulation of a compound of this disclosure and a medium generally accepted in the art for the delivery of the biologically active compound to mammals, e.g., humans. For example, a pharmaceutical composition of the present disclosure may be formulated or used as a standalone composition, or as a component in a prescription drug, an over the counter (OTC) medicine, a botanical drug, an herbal medicine, a natural medicine, a homeopathic agent, or any other form of health care product reviewed and approved by a government agency. Exemplary nutraceutical compositions of the present disclosure may be formulated or used as a standalone composition, or as a nutritional or bioactive component in food, a functional food, a beverage, a bar, a food flavor, a medical food, a dietary supplement, or an herbal product. A medium generally accepted in the art includes all pharmaceutically or nutraceutically acceptable carriers, diluents or excipients therefor.

As used herein, “enriched for” refers to a plant extract or other preparation having at least a two-fold up to about a 1000-fold increase of one or more active compounds as compared to the amount of one or more active compounds found in the weight of the plant material or other source before extraction or other preparation. In certain embodiments, the weight of the plant material or other source before extraction or other preparation may be dry weight, wet weight, or a combination thereof.

As used herein, “major active ingredient” or “major active component” refers to one or more active compounds found in a plant extract or other preparation or enriched for in a plant extract or other preparation, which is capable of at least one biological activity. In certain embodiments, a major active ingredient of an enriched extract will be the one or more active compounds that were enriched in that extract. Generally, one or more major active components will impart, directly or indirectly, most (i.e., greater than 50%, or 20% or 10%) of one or more measurable biological activities or effects as compared to other extract components. In certain embodiments, a major active ingredient may be a minor component by weight percentage of an extract (e.g., less than 50%, 25%, or 10% or 5% or 1% of the components contained in an extract) but still provide most of the desired biological activity. Any composition of this disclosure containing a major active ingredient may also contain minor active ingredients that may or may not contribute to the pharmaceutical or nutraceutical activity of the enriched composition, but not to the level of major active components, and minor active components alone may not be effective in the absence of a major active ingredient.

“Effective amount” or “therapeutically effective amount” refers to that amount of a compound or composition of this disclosure which, when administered to a mammal, such as a human, is sufficient to effect treatment, including any one or more of: (1) maintaining articular cartilage homeostasis; (2) balancing chondrocytes catabolic and anabolic process; (3) treating or preventing loss of cartilage in a mammal; (4) promoting joint health; (5) suppressing loss of cartilage in a mammal; (6) increasing joint flexibility in a mammal; (7) treating or preventing joint pain in a mammal; (8) modifying inflammation of a joint in a mammal; and (9) increasing joint range of motion, (10) managing and/or treating osteoarthritis and/or rheumatoid arthritis, preventing osteoarthritis and/or rheumatoid arthritis, or reversing the progression of osteoarthritis and/or rheumatoid arthritis in a mammal. The amount of a compound, an extract or a composition of this disclosure that constitutes a “therapeutically effective amount” will vary depending on the bioactive compound, or the biomarker for the condition being treated and its severity, the manner of administration, the duration of treatment, or the age of the subject to be treated, but can be determined routinely by one of ordinary skill in the art having regard to his own knowledge and to this disclosure. In certain embodiments, “effective amount” or “therapeutically effective amount” may be demonstrated as the quantity over the body weight of a mammal (i.e., 0.005 mg/kg, 0.01 mg/kg, or 0.1 mg/kg, or 1 mg/kg, or 10 mg/kg, or 50 mg/kg, or 100 mg/kg, or 200 mg/kg, or 500 mg/kg). The human equivalent daily dosage can be extrapolated from the “effective amount” or “therapeutically effective amount” in an animal study by utilization of FDA guideline in consideration the difference of total body areas and body weights of animals and human.

“Dietary supplements” as used herein are a product that improves, promotes, increases, manages, controls, maintains, optimizes, modifies, reduces, inhibits, or prevents a particular condition associated with a natural state or biological process, or a structural and functional integrity, a homeostasis of a biological function or a phenotypic condition (i.e., are not used to diagnose, treat, mitigate, cure, or prevent disease). For example, with regard to joint health-related conditions, dietary supplements may be used to maintain joint cartilage, minimize cartilage degradation, promote health joints by protecting cartilage integrity, diminish the action of enzymes that affect joint health, improve joint movement and/or function, alleviate joint pain, alleviate joint stiffness, improve joint range of motion and/or flexibility, promote mobility, balance anabolic and catabolic homeostasis and/or the like. In certain embodiments, dietary supplements are a special category of food, functional food, medical food and are not a drug.

“Treating” or “treatment” as used herein refers to the treatment of the disease or condition of interest in a mammal, such as a human, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving or modifying the disease or condition, i.e., causing regression of the disease or condition; or (iv) relieving the symptoms resulting from the disease or condition, (e.g., relieving pain, reducing inflammation, reducing loss of cartilage) without addressing the underlying disease or condition; (v) balancing the anabolic and catabolic homeostasis or changing the phenotype of the disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

As used herein, “statistical significance” refers to a p value of 0.050 or less when calculated using the Students t-test and indicates that it is unlikely that a particular event or result being measured has arisen by chance.

For the purposes of administration, the compounds of the present disclosure may be administered as a raw chemical or may be formulated as pharmaceutical or nutraceutical compositions. Pharmaceutical or nutraceutical compositions of the present disclosure comprise a compound of structures described in this disclosure and a pharmaceutically or nutraceutically acceptable carrier, diluent or excipient. The compound of structures described here are present in the composition in an amount which is effective to treat a particular disease or condition of interest—that is, in an amount sufficient promote chondrocyte, or extracellular matrix, or cartilage homeostasis or any of the other associated indications described herein, and generally with acceptable toxicity to a patient.

Administration of the compounds or compositions of this disclosure, or their pharmaceutically or nutraceutically acceptable salts, in pure form or in an appropriate pharmaceutical or nutraceutical composition, can be carried out via any of the accepted modes of administration of agents for serving similar utilities. The pharmaceutical or nutraceutical compositions of this disclosure can be prepared by combining a compound of this disclosure with an appropriate pharmaceutically or nutraceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, creams, lotions, tinctures, sashay, ready to drink, masks, microspheres, and aerosols. Typical routes of administering such pharmaceutical or nutraceutical compositions include oral, topical, transdermal, inhalation, parenteral, sublingual, buccal, rectal, vaginal, or intranasal. The term parenteral as used herein includes subcutaneous injections, intravenous, intramuscular, intrasternal injection or infusion techniques. Pharmaceutical or nutraceutical compositions of this disclosure are formulated so as to allow the active ingredients contained therein to be bioavailable upon administration of the composition to a patient. Compositions that will be administered to a subject or patient or a mammal take the form of one or more dosage units, where for example, a tablet may be a single dosage unit, and a container of a compound or an extract or a composition of 2-3 plant extracts of this disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of this disclosure, or a pharmaceutically or nutraceutically acceptable salt thereof, for treatment of a disease or condition of interest in accordance with the teachings of this disclosure.

A pharmaceutical or nutraceutical composition of this disclosure may be in the form of a solid or liquid. In one aspect, the carrier(s) are particulate, so that the compositions are, for example, in tablet or in powder form. The carrier(s) may be liquid, with the compositions being, for example, oral syrup, injectable liquid or an aerosol, which is useful in, for example, inhalatory administration.

When intended for oral administration, the pharmaceutical or nutraceutical composition is in either solid cream, suspension and gel forms are included within the forms considered herein as either solid or liquid.

As a solid composition for oral administration, the pharmaceutical or nutraceutical composition may be formulated into a powder, granule, compressed tablet, pill, capsule, chewing gum, sashay, wafer, bar, or like form. Such a solid composition will typically contain one or more inert diluents or edible carriers. In addition, one or more of the following may be present: binders such as carboxymethylcellulose, ethyl cellulose, cyclodextrin, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical or nutraceutical composition is in the form of a capsule, for example, a gelatin capsule, it may contain, in addition to materials of the above type, a liquid carrier such as polyethylene glycol or oil.

The pharmaceutical or nutraceutical composition may be in the form of a liquid, for example, an elixir, tincture, syrup, solution, emulsion or suspension. The liquid may be for oral administration or for delivery by injection, as two examples. When intended for oral administration, a useful composition contains, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical or nutraceutical compositions of this disclosure, whether they be solutions, suspensions or other like form, may include one or more of the following adjuvants: sterile diluents such as water for injection, saline solution, such as physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a generally useful adjuvant. An injectable pharmaceutical or nutraceutical composition is sterile.

A liquid pharmaceutical or nutraceutical composition of this disclosure intended for either parenteral or oral administration should contain an amount of a compound of this disclosure such that a suitable dosage will be obtained.

The pharmaceutical or nutraceutical composition of this disclosure may be intended for topical administration, in which case the carrier may suitably comprise a solution, emulsion, cream, lotion, ointment, or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical or nutraceutical composition for topical administration. If intended for transdermal administration, the composition may include a transdermal patch or iontophoresis device.

The pharmaceutical or nutraceutical composition of this disclosure may be intended for rectal administration, in the form, for example, of a suppository, which will melt in the rectum and release the drug. The composition for rectal administration may contain an oleaginous base as a suitable nonirritating excipient. Such bases include lanolin, cocoa butter and polyethylene glycol.

The pharmaceutical or nutraceutical composition of this disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical or nutraceutical composition of this disclosure in solid or liquid form may include an agent that binds to the compound of this disclosure and thereby assists in the delivery of the compound. Suitable agents that may act in this capacity include a monoclonal or polyclonal antibody, a protein or a liposome.

The pharmaceutical or nutraceutical composition of this disclosure in solid or liquid form may include reducing the size of a particle to, for example, improve bioavailability. The size of a powder, granule, particle, microsphere, or the like in a composition, with or without an excipient, can be macro (e.g., visible to the eye or at least 100 μm in size), micro (e.g., may range from about 100 μm to about 100 nm in size), nano (e.g., may no more than 100 nm in size), and any size in between or any combination thereof to improve size and bulk density.

The pharmaceutical or nutraceutical composition of this disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of this disclosure may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation, may determine the most appropriate aerosol(s).

The pharmaceutical or nutraceutical compositions of this disclosure may be prepared by methodology well known in the pharmaceutical or nutraceutical art. For example, a pharmaceutical or nutraceutical composition intended to be administered by injection can be prepared by combining a compound of this disclosure with sterile, distilled, deionized water so as to form a solution. A surfactant may be added to facilitate the formation of a homogeneous solution or suspension. Surfactants are compounds that non-covalently interact with the compound of this disclosure so as to facilitate dissolution or homogeneous suspension of the compound in the aqueous delivery system.

The compounds of this disclosure, or their pharmaceutically or nutraceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of this disclosure, or pharmaceutically or nutraceutically acceptable derivatives thereof, may also be administered simultaneously with, prior to, or after administration of food, water and one or more other therapeutic agents. Such combination therapy includes administration of a single pharmaceutical or nutraceutical dosage formulation which contains a compound or an extract or a composition with 2-3 plant extracts of this disclosure and one or more additional active agents, as well as administration of the compound or an extract or a composition with 2-3 plant extracts of this disclosure and each active agent in its own separate pharmaceutical or nutraceutical dosage formulation. For example, a compound or an extract or a composition with 2-3 plant extracts of this disclosure and another active agent can be administered to the patient together in a single oral dosage composition, such as a tablet or capsule, or each agent can be administered in separate oral dosage formulations. Where separate dosage formulations are used, the compounds of this disclosure and one or more additional active agents can be administered at essentially the same time, i.e., concurrently, or at separately staggered times, i.e., sequentially; combination therapy is understood to include all these regimens.

It is understood that in the present description, combinations of substituents or variables of the depicted formulae are permissible only if such contributions result in stable compounds.

It will also be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of this disclosure which are pharmacologically active. Such derivatives may therefore be described as “prodrugs”. All prodrugs of compounds of this disclosure are included within the scope of this disclosure.

Furthermore, all compounds or extracts of this disclosure which exist in free base or acid form can be converted to their pharmaceutically or nutraceutically acceptable salts by treatment with the appropriate inorganic or organic base or acid by methods known to one skilled in the art. Salts of the compounds of this disclosure can be converted to their free base or acid form by standard techniques.

In some embodiments, compounds or extracts of the present disclosure can be isolated from plant sources, for example, from those plants included in the Examples and elsewhere throughout the present application. Suitable plant parts for isolation of the compounds include leaves, bark, trunk, trunk bark, stem, stem bark, twigs, tubers, root, rhizome, root bark, bark surface, young shoots, seed, fruit, androecium, gynoecium, calyx, stamen, petal, sepal, carpel (pistil), flower, or any combination thereof. In some related embodiments, the compounds or extracts are isolated from plant sources and synthetically modified to contain any of the recited substituents. In this regard, synthetic modification of the compound isolated from plants can be accomplished using any number of techniques which are known in the art and are well within the knowledge of one of ordinary skill in the art.

Kochia scoparia, also identified as Bassia scoparia; Bassia sieversiana; Kochia alata; Kochia trichophila; Kochia trichophylla, is a large annual herb growing from seeds with the common names: burning bush, ragweed, summer cypress, kochia and Mexican fireweed. The plant is native to Asia but naturalized in many parts of North America as well. Kochia scoparia plant contains high levels of protein and is commonly used as forage for livestock. The seeds could be served as food for birds and are also valuable as poultry feed. Kochia seeds are also used as food garnish in Japan called Tonburi or land caviar.

Kochiae fructus or seeds has been used as folk medicine in Asian countries to treat a variety of diseases, such as skin diseases, diabetes mellitus, rheumatoid arthritis, liver disorders, and jaundice, etc. Recent studies have also reported kochia seeds with antioxidant, anti-inflammatory, antiparasitic, anti-cancer, antidiabetic, hypoglycemic, weight loss, anti-allergic, analgesic properties. Oleanolic acid type triterpenoid saponins were identified as the active components responsible for most of Kochia fructus efficacies. Momordin Ic, originally isolated from Momordica cochinchinensis, is a principle constituent of Kochiae fructus and is also reported in various natural herbal medicines with antinociceptive and anti-inflammatory activities in hind paw licking and formalin test in mice. Both 70% Kochiae fructus ethanol extracts and Momordin Ic showed inhibitory effects in Carrageenan-induced paw edema model in mice.

Kochia extract, as demonstrated in example 1 to 4, is a contemplated component or constituent that can be utilized as part of a target compound or composition. Kochia extract may be obtained from any suitable source, including Kochia scoparia, Bassia scoparia, Bassia angustifolia, Momordica cochinchinensis, Bassia dinteri, Bassia eriophora, Bassia hyssopifolia, Bassia indica, Bassia lanflora, Bassia lasiantha, Bassia littorea, Bassia muricata, Bassia odontoptera, Bassia pilosa, Bassia prostrata, Bassia salsoloides, Bassia stellaris, Bassia tianschanica, Bassia tomentosa, Bassia villosissima or a combination thereof.

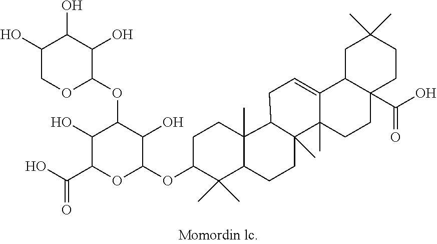 [SMILES: CC1(C)CCC2(C(=O)O)CCC3(C)C(=CCC4C5(C)CCC(OC6OC(C(=O)O)C(O)C(OC7OCC(O)C(O)C7O)C6O)C(C)(C)C5CCC43C)C2C1]

As illustrated in examples 1, 2, 3 and 4, Kochia extract may be enriched for one or more as contemplated herein. Contemplated saponins isolated from Kochia scoparia that are extracted with any suitable solvent, including supercritical fluid of CO₂, water, methanol, ethanol, alcohol, a water-mixed solvent or a combination thereof, or with supercritical fluid. In contemplated embodiments, the Kochia extract comprises about 0.01% to about 99.9% saponins. Contemplated saponins isolated from Kochia extract are Bassiasaponin A; Bassiasaponin B; Kochioside A; Kochioside B; Kochioside C; Kochianoside I; Scoparianos A; Scoparianoside B; Scoparianoside C; Momordin Ic; Kochianoside I; Kochianoside II; Kochianoside III; Kochianoside IV; 2′-O-Glucopyranosylmomordin Ic; 2′-O-Glucopyranosylmomordin IIc, etc.

Alpinia galanga belongs to the ginger family and is used as a spice herb in southeast Asian cuisine with the common names: lengkuas, greater galangal and blue ginger. The plant is a perennial herb native to southeast Asia and found commonly in greater Sunda islands, Philippines, and Thailand. This plant's rhizome has been used for food with a unique pungent sensation similar to black pepper without a lingering effect. Alpinia galanga as traditionally used to treat various kinds of disease including eczema, bronchitis, otitis internal, gastritis, ulcers and cholera, appetite boosting, tonic effect, etc. Many pharmacological activities have been reported for Alpinia galanga, especially as antibacterial, antifungal, antiviral, immunomodulatory, antioxidant, antidiabetic, analgesic and many other pharmacological functions.

The main chemical constituents of Alpinia galanga are reported as flavonoids, such as kaempferol, kaempferide, and volatile components including trans-p-coumaryl diacetate, di-(p-hydroxy-cis-styryl) methane, eugenol acetate, l-hydroxychavicol acetate, p-hydroxycinnamaldehyde, etc. Phenylpropanoids, l′-acetoxychavicol acetate (galangal acetate) and trans p-coumaryl diacetate, were isolated and identified as two main active compounds of this plant in this study. l′-acetoxychavicol acetate was reported as pungent principle for Alpinia galanga and was reported to have antimicrobial and anticancer properties as well.

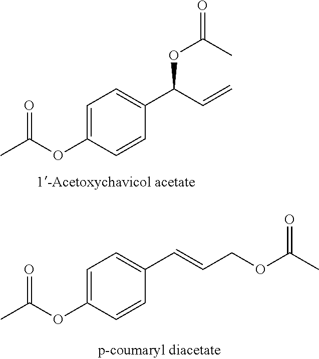

The anti-inflammatory and analgesic activities of the topical application of Alpinia galanga methanolic extract were illustrated in the examples 8, 9, 10 and 11. The anti-inflammatory and analgesic activity of Alpinia methanol extract was also found in both Carrageenan-induced paw edema model in rats and in formalin test. One of major phenylpropanoids, 1′-Acetoxychavicol acetate, and Alpinia galanga acetone extract have been reported effective in Incomplete Freund's Adjuvant (IFA)-induced arthritis model in rats.

Alpinia extract is a contemplated component or constituent that can be utilized as part of a target compound or composition. Alpinia extract may be obtained from any suitable galangal source, including Alpinia galanga, Alpinia officinarum, Boesenbergia rotunda, Kaempferia galanga, Alpinia oxyphylla, Alpinia abundiflora, Alpinia acrostachya, Alpinia caerulea, Alpinia calcarata, Alpinia conchigera, Alpinia globosa, Alpinia javanica, Alpinia melanocarpa, Alpinia mutica, Alpinia nigra, Alpinia nutans, Alpinia petiolate, Alpinia purpurata, Alpinia pyramidata, Alpinia rafflesiana, Alpinia speciosa, Alpinia vittata, Alpinia zerumbet, Alpinia zingiberina, or a combination thereof.

Alpinia extract may be enriched for one or more as contemplated herein and as demonstrated in examples 8, 9, 10 and 11. Contemplated aromatics isolated from Alpinia extract are extracted with any suitable solvent, including supercritical fluid of CO₂, water, methanol, ethanol, alcohol, a water-mixed solvent, organic solvent, such as hexane, ethyl acetate, acetone, butanol; or a combination thereof, or with supercritical fluid, or by water distillation of oil in the rhizomes. In contemplated embodiments, the Alpinia extract comprises about 0.01% to about 99.9% phenylpropanoid small aromatics. Contemplated aromatics isolated from Alpinia extract are 1′-Acetoxyeugenol acetate; Coniferyl diacetate; 3-(4-Hydroxyphenyl)-2-propenal; 3-(4-Hydroxyphenyl)-2-propen-1-ol; Methyl cinnamate, FEMA 2698; 3-(4-Methoxyphenyl)-2-propen-1-ol; l-Hydroxychavicol acetate; 4-Acetoxycinnamyl alcohol; 4-Acetoxycinnamyl ethyl ether; 1′-Ethoxychavicol acetate; 1-(3,4-Dihydroxyphenyl)-2-propen-1-ol; (S)-form, 3′-Me ether, 4′-Ac; 1′-Acetoxychavicol acetate; 1-(4-Hydroxyphenyl)-2-propen-1-ol; -form, Di-Ac; 3-(4-Hydroxyphenyl)-2-propen-1-ol; (E)-form, Di-Ac; 4-(2-Propenyl)-1,2-benzenediol; 1-O-D-Glucopyranoside; 4-(2-Propenyl)-1,2-benzenediol; 2-O-D-Glucopyranoside; Bis(4-acetoxycinnamyl) ether; Ethyl 4-feruloyl-D-glucopyranoside; Lusitanicoside; 4-(2-Propenyl)-1,2-benzenediol; 1-O-[-L-Rhamnopyranosyl-D-glucopyranoside]; 4-(2-Propenyl)-1,2-benzenediol; Di-O-D-glucopyranoside; 4′-O-trans-Feruloyltachioside or a combination thereof.

Piper nigrum, with common name black pepper, is a flowing vine of the family Piperaceae. In this disclosure, the terms “Piper”, “Pepper”, and “Piper/Pepper” are used interchangeably to refer to embodiments comprising this extract or constituent. Black pepper is native to Kerala state in Southwestern India and extensively cultivated in tropical regions, such as Vietnam, India, and Indonesia. The ground dried fruit, known as peppercorn, has been used for its flavor and as traditional medicine. Black pepper is one of the most commonly used spices in the world. Piperine is the main constituent in black pepper contributing to the hot and pungent flavor.

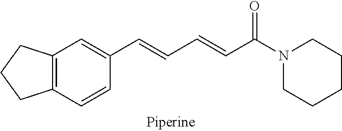 [SMILES: O=C(/C=C/C=C/c1ccc2c(c1)CCC2)N1CCCCC1]

Black peppercorns feature as remedies in Ayurveda, Siddha and Unani medicine in South Asia. They are used as an appetizer and to treat digestive system-related problems. Black pepper could be used as a remedy for sore throat to reduce throat inflammation. Externally, it could be applied to reduce hair loss and treat some skin problems. Many pharmacological effects have been reported for black pepper, such as antifungal, antioxidant, digestive boosting, anti-depressant and cognitive effect, analgesic and anti-inflammatory, anticancer, immuno-modulatory, lipid lowering, etc.

Piper extract is a contemplated component or constituent that can be utilized as part of a target compound or composition. Piper extract may be obtained from any suitable source as illustrated in example 5, 6, and 7, including Piper nigrum and many other Piper spp., Periconia sp. Piper nigrum, Piper longum, Piper amalgo, Piper aurantiacum, Piper chaba, Piper capense, Piper crassinervium, Piper guineense, Piper methysticum, Piper novae-hollandiae, Piper peepuloides, Piper ponapense, Piper puberulum, Piper retrofractum, Piper sintenense, Piper tuberculatum, Piper hancei, Glycine max, Petrosimonia monandra, Mentha piperata, silocaulon absimile, and Ulocladium sp or a combination thereof.

The principle active alkaloid compound, Piperine, was extensively studied and reported to act as a central nervous system antidepressant and nerve stimulant, and to have antioxidant, anti-fever, hepatoprotective, pain-relieving, anti-inflammatory, insecticidal and many other effects. Piperine was also reported as a bioavailability enhancer.

Piper extract may be enriched for one or more as contemplated herein as illustrated in examples 5, 6, and 7. Contemplated alkaloids isolated from Piper extract are extracted with any suitable solvent, including supercritical fluid of CO₂, water, methanol, ethanol, alcohol, a water-mixed solvent, organic solvent such as ethyl acetate, acetone, butanol or a combination thereof, or with supercritical fluid. In contemplated embodiments, the Piper extract comprises about 0.01% to about 99.9% piperidine alkaloids. Contemplated alkaloids isolated from Piper extract are Piperine; Piperchabamide A; Kaousine; 5-Acetoxy-5,6-dihydro-1-(3-phenylpropanoyl)-2(1H)-pyridinone; 5,6-Dihydro-N-(3,4-dimethoxycinnamoyl)-2(1H)-pyridinone; N-[3-(3,4-Dimethoxyphenyl)propanoyl]-5,6-dihydro-2(1H)-pyridinone; Cenocladamide; 3,4-Epoxypipermethystine; 4′-O-Demethylpiplartine; Piplaroxide; cis-Piplartine; Piplartine; 8,9-Dihydropiplartine; 3,4-Epoxy-8,9-dihydropiplartine; Cycloguineense B; Nigramide K; Nigramide H; Nigramide J; Nigramide M; Nigramide N; Nigramide I; Nigramide L; Chabamide H; Chabamide I; Nigramide Q; Nigramide A; Nigramide C; Nigramide P; Dipiperamide C; Nigramide G; Dipiperamide A; Dipiperamide E; Pipercyclobutanamide A; Nigramide R; Nigramide B; Dipiperamide B; Dipiperamide F; Dipiperamide G; Nigramide F, Piperchabamide G; Piperarborenine A; Piplartine dimer A; 7,8′-Diepimer, 3,3′-bis(demethoxy); 1,1′-[[2,4-Bis(6-methoxy-1,3-benzodioxol-5-yl)-1,3-cyclobutanediyl]dicarbonyl]bispiperidine; Piperarborenine E; Pipercyclobutanamide B; Dipiperamide D; Nigramide S; Nigramide D; Nigramide E; Piperarborenine D; Piperarborenine B; 2,4-Bis(2-methoxy-4,5-methylenedioxyphenyl)-1,3-cyclobutanecarboxylic acid dipiperidide; 3′-Methoxy; Piperarborenine C; Piperarboresine; Piperchabamide H; 1,1′-[[2,4-Bis(3,4,5-trimethoxyphenyl)-1,3-cyclobutanediyl] dicarbonyl]bis[5,6-dihydro-2(1H)-pyridone], 3-Phenylpropanoic acid 2,3-didehydro-4-hydroxypiperidide; 1-(1,6-Dioxo-2,4-decadienyl)piperidine; 1-[5-(4-Hydroxyphenyl)-1-oxo-2,4-pentadienyl]piperidine; Ilepcimide; 3,4-Methylenedioxycinnamoyl piperidide; (Z)-form; 3,4-Dihydroxy-1-(3-phenylpropanoyl)-2-piperidinone; 4,5-Dihydroxy-2-decenoic acid piperidide; 4,5-Dihydroxy-2-decenoic acid; (2E,4S,5R)-form, Piperidide; Chavicine; Isochavicine; Isopiperine; Piperpense; Feruperine; 1-[5-(1,3-Benzodioxol-5-yl)-1-oxo-2-pentenyl]piperidine; N-(3-Methoxy-4,5-methylenedioxycinnamoyl)piperidide; 2-Methoxy-4,5-methylenedioxycinnamoyl piperidide; 2-Hydroxy-4,5-methylenedioxycinnamic acid; (Z)-form, Me ether, piperidide; Dihydroferuperine; Tetrahydropiperine; Piperlongumamide C; Puberullumine; 1-[7-(1,3-Benzodioxol-5-yl)-1-oxo-2,4,6-heptatrienyl]piperidine; 1-[7-(1,3-Benzodioxol-5-yl)-1-oxo-2,4-heptadienyl]piperidine; Pipersintenamide; Wisanine; (E,E)-form; Piperx; Piperolein A; (E)-form; Piperine S; Piperodione; 4,5-Dihydro-2′-methoxypiperine; 2,4-Hexadecadienoic acid piperidide; Piperlongimine B; 11-Phenyl-2,4-undecadienoic acid piperidide; Piperlongumamide B; 1-[8-(1,3-Benzodioxol-5-yl)-1-oxo-7-octadecenyl]piperidine; Dehydropipernonaline; Piptigrine; 1-[9-(1,3-Benzodioxol-5-yl)-1-oxo-2,8-nonadienyl]piperidine; Piperolein B; Piperoctadecalidine; 2,4,12-Octadecatrienoic acid piperidide; 2,4-Octadecadienoic acid piperidide; 1-[11-(1,3-Benzodioxol-5-yl)-1-oxo-2,4,10-undecatrienyl]piperidine; Piperchabamide B; Pipereicosalidine; 1-[8,9-Dihydroxy-9-(3,4-methylenedioxyphenyl)-2-nonenoyl]piperidine; Pipernonaline; 8,9-Dihydro, 8R*,9S*-dihydroxy; N-(2,14-Eicosadienoyl)piperidine; 2,4-Eicosadienoic acid piperidide; 1-[13-(1,3-Benzodioxol-5-yl)-1-oxo-2,4,12-tridecatrienyl]piperidine; Pipertridecadienamide; Pipsaeedine; Pipbinineor; or a combination thereof.

Magnolia officinalis, commonly known as “houpu” in Chinese as one of the most popular traditional Chinese medicine plants, with a very wide range of applications. It is a species of Magnolia that is native in China, mainly growing in Sichuan and Hubei provinces. Houpu refers to its thick bark, which can be stripped from the stems, branches, and roots. The traditional indications are to treat wind stroke, cold damage, headache, fight qi and blood impediments. Magnolia bark has been used to treat menstrual cramps, abdominal pain, abdominal bloating and gas, nausea, and indigestion. The bark is also an ingredient in formulas used for treating coughs and asthma. Many of the formulations with Magnolia bark are used in treating lung diseases such as including cough and asthma or intestinal infections and spasms, relieving abdominal swelling of various causes and edema.

Bisphenolic lignans are identified as the major active components responsible for the efficacy. Magnolol and honokiol, as two main polyphenol compounds found in Magnolia bark, have been reported with various pharmacological activities and functions, such as antioxidant, anti-inflammatory, and antitumor (Park 2004). The anticancer studies of honokiol have been extended to several different solid tumor types such as breast, prostate, gastric, and ovarian cancer, with potential to enhance current anticancer regimens (Fried 2009). Honokiol also reduced inflammation and oxidative stress, providing beneficial effects in neurological protection, and glucose regulation with great potential as therapeutic agents for inflammatory disease. In particular, magnolol and honokiol have been known to exhibit potent antimicrobial activity against Gram-positive and Gram-negative bacteria as well as fungi such as Propionibacterium sp. and S. aureus showing its potential as antimicrobial agents effective against more infectious and antibiotic resistant microorganisms (Bopaiah 2001; Bang 2000; Syu 2004). The content of honokiol and magnolol could be varied from 1-99% in the commercialized Magnolia bark extracts.

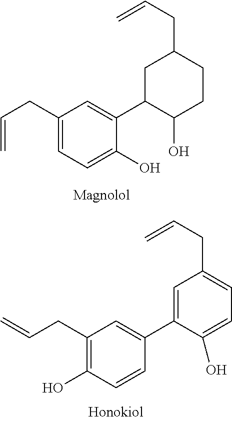 [SMILES: C=CCc1ccc(O)c(C2CC(CC=C)CCC2O)c1 ; C=CCc1ccc(O)c(-c2ccc(O)c(CC=C)c2)c1]

### Magnolol and Honokiol

As demonstrated in example 13, Magnolia extract is a contemplated component or constituent that can be utilized as part of a target compound or composition. Magnolia extract may be obtained from any suitable source, including Magnolia officinalis, Magnolia acuminate, Magnolia biondii, Magnolia coco, Magnolia denudate, Magnolia fargesii, Magnolia garrettii, Magnolia grandiflora, Magnolia henryi, Magnolia liliflora, Magnolia kachirachirai, Magnolia Kobus, Magnolia obovata, Magnolia praecocissima, Magnolia pterocarpa, Magnolia pyramidata, Magnolia rostrate, Magnolia salicifolia, Magnolia sieboldii, Magnolia soulangeana, Magnolia stellate, Magnolia virginiana, prod. of degradation of birch lignin, Acanthus ebracteatus, Aptosimum spinescens, Aralia bipinnata, Araucaria angustifolia, Araucaria araucana, Artemisia absinthium, Haplophyllum acutifolium, Haplophyllum perforatum, Liriodendron tulipifera, Krameria cystisoides, Perilla frutescens, Lawsonia inermis Myristica fragrans (nutmeg), Parakmeria yunnanensis (preferred genus name Magnolia), Persea japonica, Piper futokadsura, Piper wightii, Rollinia mucosa, Sassafras randaiense, Scrophularia albida-colchica, Stellera chamaejasme, Syringa velutina, Syzygium cumini, Talauma gloriensis, Virola elongate, Urbanodendron verrucosum, Wikstroemia sikokiana or a combination thereof.

Magnolia extract may be enriched for one or more as contemplated herein. Contemplated lignans isolated from Magnolia extract are extracted with any suitable solvent, including supercritical fluid of CO₂, water, methanol, ethanol, alcohol, organic solvent such as ethyl acetate, acetone, butanol; a water-mixed solvent or a combination thereof, or with supercritical fluid. In contemplated embodiments, the Magnolia extract comprises about 0.01% to about 99.9% biphenolic lignans. Contemplated lignans isolated from Magnolia extract are magnolol, honokiol, Magnaldehyde D; Magnaldehyde D; 4′-Deoxy, 6′-hydroxy; 6,8-Epoxy-3,3′-ligna-7,8′-dien-4′-ol; 3,3′-Ligna-8,8′-diene-4,6′-diol; 3,3′-Ligna-8,8′-diene-4,4′-diol; 3,3′-Ligna-8,8′-diene-4,6′-diol; 7′-Isomer(E-); Magnaldehyde D; 6′-Methoxy, 4′-deoxy; Magnaldehyde A; Magnaldehyde A; 6′-Hydroxy, 4′-deoxy; 6,8-Epoxy-3,3′-ligna-7,8′-dien-4′-ol; 9-Hydroxy; 3,3′-Ligna-8,8′-diene-4,6′-diol; 6′-Me ether; 3-Formyl-2,2′-dihydroxy-5,5′-di-2-propenylbiphenyl; Magnaldehyde A; 6′-Methoxy, 4′-deoxy; Magnaldehyde A; 6-Methoxy, 4-deoxy; 3,3′-Ligna-8,8′-diene-4,4′-diol; 4-Et ether; 3,3′-Ligna-8,8′-diene-4,4′,5-triol; 5-Me ether; Magnolignan E; Magnolignan C; Magnolignan A; 8′,9′-Dihydroxyhonokiol; 3,3′-Ligna-8,8′-diene-4,4′-diol; 4-O-(2-Propenyl) ether; 4-Hydroxy-6′-methoxy-3,3′-ligna-7,7′-diene-9,9′-dial; Magnaldehyde C; threo-Honokitriol; erythro-Honokitriol; threo-Magnolignan B; erythro-Magnolignan B; Coumanolignan; Magnolignan D; erythro-Magnolignan D; 5,5′-Diallyl-2′-(3-methyl-2-butenyloxy)biphenyl-2-ol; 7-O-Ethylhonokitriol; 6′-Amino-3,3′-ligna-8,8′-dien-6-ol; N-[2-(4-Hydroxyphenyl)ethyl]; Houpulin C; Piperitylmagnolol; Piperitylhonokiol; Bornylmagnolol; Houpulin I; Houpulin F; Houpulin G; Houpulin H; Magnolignan A 4′-glucoside; Magnolignan C 6′-glucoside; Clovanemagnolol; Eudeshonokiol A; Eudeshonokiol B; Eudesmagnolol or a combination thereof.

Contemplated compounds, medicinal compositions and compositions may comprise or additionally comprise or consist of at least one active ingredient. In some embodiments, at least one bioactive ingredient may comprise or consist of plant powder or plant extract or the like.

In any of the aforementioned embodiments, the compositions comprising mixtures of extracts or compounds may be mixed at a particular ratio by weight. Demonstrated in example 15, an Alpinia extract and a Pepper extract may be blended in a 1:2 weight ratio, respectively. In certain embodiments, the ratio (by weight) of two extracts or compounds of this disclosure ranges from about 0.5:5 to about 5:0.5. Similar ranges apply when more than two extracts or compounds (e.g., three, four, five) are used. Exemplary ratios include 0.5:1, 0.5:2, 0.5:3, 0.5:4, 0.5:5, 1:1, 1:2, 1:3, 1:4, 1:5, 2:1, 2:2, 2:3, 2:4, 2:5, 3:1, 3:2, 3:3, 3:4, 3:5, 4:1, 4:2, 4:3, 4:4, 4:5, 5:1, 5:2, 5:3, 5:4, 5:5, 1:0.5, 2:0.5, 3:0.5, 4:0.5, or 5:0.5. In certain embodiments illustrated in example 14, the disclosed individual extracts of Alpinia, and/or Pepper, and/or Magnolia and/or Kochia are blended into a composition with 3 individual extracts in a 1:1:1, 2:1:1, 3:1:1, 4:1:1, 5:1:1, 1:2:1, 1:3:1, 1:4:1, 1:5:1, 1:1:2, 1:1:3, 1:1:4, 1:1:5, 1:2:3, 1:2:4, 1:2:5, 1:2:6, 1:2:6, 1:2:8, 1:2:9 or 1:2:10 etc. weight ratio, respectively. In further embodiments, the disclosed individual extracts of Alpinia, Pepper, Magnolia and Kochia have been combined into a composition called AMK as an examples but not limited to a blending ratio of 2:4:3 and 5:4:4 as of Alpinia:Magnolia:Kochia as demonstrated in example 14. In further embodiments, such combinations of individual extracts of Alpinia, Pepper, Magnolia and Kochia at various combinations of 2 to 3 of those extracts with examples, but not limited to, Alpinia:Pepper (AP) and Alpinia:Magnolia:Kochia (AMK), were evaluated on in vitro, and/or ex vivo and/or in vivo models for advantage/disadvantage and unexpected synergy/antagonism of the perceived biological functions and effective adjustments of anabolic and catabolic homeostasis of chondrocytes, extracellular matrix, articular cartilage, and phenotype of arthritis. The best compositions with specific blending ratio of individual extracts of Alpinia, or Pepper, or Magnolia or Kochia were selected based on unexpected synergy measured on the in vitro, and/or ex vivo and/or in vivo models due to the diversity of chemical components in each extract and different mechanism of actions from different types of bioactive compounds in each extract, and potential enhancement of ADME of natural compounds in the composition to maximize the biological outputs.

In any of the aforementioned embodiments, the compositions comprising mixtures of extracts or compounds may be present at certain percentage levels or ratios. In certain embodiments, a composition comprising an Alpinia extract and/or a Kochia extract can include 0.1% to 49.9% or about 2% to about 40% or about 0.5% to about 8% of acetoxychavicol acetate, 0.1% to 49.9% or about 1% to about 10% or about 0.5% to about 3% of Momordin lc, or a combination thereof. In certain embodiments, a composition comprising an Alpinia extract can include from about 0.01% to about 99.9% acetoxychavicol acetate or include at least 1%, 2%, 3%, 4%, 5% 6%, 7%, 8%, 9%, 10%, 11%, 12%, 13%, 14%, 15%, 16%, 17%, 18%, 19%, 20%, 30%, 35%, 40%, 45%, 50%, 55%, 60%, 65%, 70%, 75%, or 80% acetoxychavicol acetate (e.g., 1′-acetoxychavicol acetate, or p-coumaryl diacetate, or both)

In certain examples, a composition of this disclosure may be formulated to further comprise a pharmaceutically or nutraceutically acceptable carrier, diluent, or excipient, wherein the pharmaceutical or nutraceutical formulation comprises from about 0.05 weight percent (wt %), or 0.5 weight percent (wt %), or 5%, or 25% to about 95 wt % of active or major active ingredients of an extract mixture. In further embodiments, the pharmaceutical or nutraceutical formulation comprises from about 0.05 weight percent (wt %) to about 90 wt %, about 0.5 wt % to about 80 wt %, about 0.5 wt % to about 75 wt %, about 0.5 wt % to about 70 wt %, about 0.5 wt % to about 50 wt %, about 1.0 wt % to about 40 wt %, about 1.0 wt % to about 20 wt %, about 1.0 wt % to about 10 wt %, about 3.0 wt % to about 9.0 wt %, about 5.0 wt % to about 10 wt %, about 3.0 wt % to about 6 wt % of the major active ingredients in an extract mixture, or the like. In any of the aforementioned formulations, a composition of this disclosure is formulated as a tablet, hard capsule, softgel capsule, powder, or granule.

Also contemplated herein are agents of the disclosed compounds. Such products may result from, for example, the oxidation, reduction, hydrolysis, amidation, esterification, and the like of the administered compound, primarily due to enzymatic processes. Accordingly, contemplated compounds are those produced by a process comprising administering a contemplated compound or composition to a mammal for a period of time sufficient to yield a metabolic product thereof. Such products are typically identified by administering a radiolabeled or not radiolabeled compound of this disclosure in a detectable dose to an animal, such as rat, mouse, guinea pig, dog, cat, pig, sheep, horse, monkey, or human, allowing sufficient time for metabolism to occur, and then isolating its conversion products from the urine, blood or other biological samples.

Contemplated compounds, medicinal compositions and compositions may comprise or additionally comprise or consist of at least one pharmaceutically or nutraceutically or cosmetically acceptable carrier, diluent or excipient. As used herein, the phrase “pharmaceutically or nutraceutically or cosmetically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. Contemplated compounds, medicinal compositions and compositions may comprise or additionally comprise or consist of at least one pharmaceutically or nutraceutically or cosmetically acceptable salt. As used herein, the phrase “pharmaceutically or nutraceutically or cosmetically acceptable salt” includes both acid addition and base addition salts.

In some embodiments, bioactives from the disclosed individual extracts of Alpinia, Piper/Pepper, Magnolia and Kochia and/or at various combinations of 2 to 3 of those extracts with examples, but not limited to, Alpinia:Piper/Pepper (AP) and Alpinia:Magnolia:Kochia (AMK), can be optionally combined with other RA and OA management agents, such as non-steroidal anti-inflammatory agents/analgesics, COX-2 inhibiting agents including, but not limited to, acetaminophen, ibuprofen, naproxen, Aspirin, Diclofenac, Indomethacin, Piroxicam, Ketoprofen, Trolamine salicylate; neuropathic pain relief agents such as Lidocaine; biological agents Methotrexate, Il-1 and TNF-α anti-bodies; herbal and/or plant extracts promoting joint health including but not limited to Cannabis sativa (Hemp Oil) oil or CBD/THC, full spectrum Hemp extract, turmeric extract or curcumin, terminalia extract, willow bark extract, Devil's claw root extract, Cayenne Pepper extract or capsaicin, Prickly Ash bark extract, Nexrutine or philodendra bark extract, Perluxan or hop extract, 5-Loxin/Apresflex or Boswellia and/or Boswellia serrata extract, Morus alba root bark extract, Acacia catechu extract, Scutellaria baicalensis root extract, rose hips extract, rosemary extract, green tea extract, sophora extract, Mentha or Peppermint extract, ginger or black ginger extract, green tea or grape seed polyphenols, bakuchiol or Psoralea seed extract, fish oil, Piascledine or ASU, or dietary supplements that promote joint health, including but not limited to glucosamine compounds such as glucosamine sulfate, glucosamine hydrochloride, N-acetylglucosamine, chondroitin chloride, chondroitin sulfate and methylsulfonylmethane (MSM), hyaluronic acid, UC—II or undenatured and/or denatured collagen, Omega-3 and/or Omega-6 Fatty Acids, Krill oil, Egg Shell Membrane (ESM), gamma-linolenic acid, Perna Canaliculus (Green-Lipped Mussel—GLM), SAMe, avocado/soybean unsaponifiable (ASU) extract, citrus bioflavonoids, Acerola concentrate, astaxanthin, pycnogenol, vitamin D, vitamin E, vitamin K, vitamin B, vitamin A, L-lysine, calcium, manganese, Zinc, and mineral amino acid chelate(s), boron and boron glycinate, silica, probiotics, Camphor, and Menthol.

Other embodiments of the disclosure relate to methods of use of the disclosed individual extracts of Alpinia, Piper/Pepper, Magnolia and Kochia and/or at various combinations of 2 to 3 of those extracts with examples, but not limited to, Alpinia:Piper/Pepper (AP) and Alpinia:Magnolia:Kochia (AMK), in this disclosure, including, but not limited to maintaining catabolic/anabolic biomarker homeostasis. Those catabolic biomarkers are, but not limited to, TNF-α, IL-1β, IL-6, aggrecanase and matrix metalloproteinase (MMP) such as MMP13, MMP9, MMP3, MMP1, uCTX-II and ADAMTS4; and those anabolic biomarkers are but not limited to SOX 9, TGF-β1, ACAN, COL2A1, and PIIANP.

Other embodiments of the disclosure relate to methods of use of the disclosed individual extracts of Alpinia, Piper/Pepper, Magnolia and Kochia and/or at various combinations of 2 to 3 of those extracts with examples, but not limited to, Alpinia:Piper/Pepper (AP) and Alpinia:Magnolia:Kochia (AMK), in this disclosure, including, but not limited to maintaining cartilage homeostasis, inducing cartilage synthesis (and hence, anabolic effect) and inhibiting the catabolic process of degradation and broken down, protecting extracellular matrix integrity, and joint cartilage, minimizing cartilage degradation, alleviating cartilage breakdown, and initiating and/or promoting and/or enhancing cartilage synthesis, cartilage renewal and cartilage rebuild, repairing damaged cartilage, maintaining, rebuilding and repairing extra cellular matrix of joint tissue, revitalizing joints structure, maintaining steady blood flow to joints, promoting health joints by protecting cartilage integrity, balancing anabolic and catabolic processes, maintaining synovial fluid for joint lubrication in a mammal, diminishing the action of enzymes and proinflammatory cytokines that affect joint health of a mammal.

Other embodiments of the disclosure relate to methods of use of the disclosed individual extracts of Alpinia, Piper/Pepper, Magnolia and Kochia and/or at various combinations of 2 to 3 of those extracts with examples, but not limited to, Alpinia:Piper/Pepper (AP) and Alpinia:Magnolia:Kochia (AMK), in this disclosure, including, but not limited to improving joint movement and/or physical function, maintaining joint health and mobility into old age, supporting, protecting or promoting joint comfort, alleviating joint pain, reducing joint friction, alleviating joint stiffness, improving joint range of motion and/or flexibility, promoting mobility, reducing inflammation, reducing oxidative stress, reducing and protecting joint wear and tear, managing and/or treating osteoarthritis and/or rheumatoid arthritis, preventing osteoarthritis and/or rheumatoid arthritis, or reversing the progression of osteoarthritis and/or rheumatoid arthritis; Preventing and treating juvenile rheumatoid arthritis, Still's disease, psoriatic arthritis, reactive arthritis, septic arthritis, Reiter's syndrome, Behcet's syndrome, or Felty's syndrome or the like of a mammal.

### EXAMPLES

### Example 1. Preparation and HPLC Quantification of Extracts from Kochia scoparia Fruits

The dry Kochia scoparia fruits were ground into powder. 20 grams of Kochia scoparia fruit powder were mixed with enough Diatomaceous earth to fill up a 100 mL extraction cell, and extracted with 100% Ethanol (EE), 70% Ethanol/water (70E) or 50% Ethanol/water (50E) by using ASE 350 Extractor (Extraction condition: Heat=5 minutes, Static=5 minutes, Flush=80 volume, Purge=900 seconds, Cycles=3, Pressure=1500 psi, Temperature=60° C.). After extraction, the solution was concentrated with a rotary evaporator at 50° C. and high vacuum to produce to a solid extract.

The target components, such as Momordin lc, in the Kochia extracts were quantified with a Luna C18 reversed-phase column (Phenomenex, 10 μm, 250 mm×4.6 mm) in a Hitachi HPLC system detected at 205 nm wavelength. The column was eluted with a binary gradient of 0.1% Trifluoroacetic acid in water (mobile phase A) and acetonitrile (mobile phase B) at 1 ml/min flow rate and 35° C. column temperature.

Reference Standard produced according to Example xx was utilized as the quantification standard. All samples were prepared in MeOH for HPLC analysis with standard in a concentration around 3 mg/ml, and extract samples in a concentration around 10 mg/ml.

TABLE 1
Gradient Table of HPLC Analytical Method

| Time (min) | Mobile phase A | Mobile phase B |
|---|---|---|
| 0.0 | 70 | 30 |
| 20 | 0 | 100 |
| 23 | 0 | 100 |
| 23.1 | 70 | 30 |
| 30 | 70 | 30 |

TABLE 2
Extraction and HPLC quantification results of Kochia extract

| | | | Momordin lc |
|---|---|---|---|
| Sample ID | Extraction solvent | Extraction Yield | in extract |
| R00577-EE | Ethanol | 17.8% | 11.2% |
| R00577-70E | 70% Ethanol/water | 22.2% | 9.7% |
| R00577-50E | 50% Ethanol/water | 19.1% | 9.9% |

### Example 2. Isolation and Purification of Momordin lc from Kochia scoparia

Ethanol extract (EE, 10 g) from fruits of Kochia scoparia was partitioned between organic solvent (100 ml each) and water (150 ml) in the order of Hexane, EtOAC and BuOH to generate Hexane fraction (HE), EtOAC fraction (EA), BuOH fraction (Bu) and water fraction (WA). The compound Momordin lc was enriched in BuOH fraction (1.7 g), the marker compound was increased from 9.7% in extract to 46.2% in BuOH fraction.

TABLE 3
Inhibition of GAG release of Kochia extract and fractions

| | Inhibition of GAG release | | | |
|---|---|---|---|---|
| Sample ID | 25 μg/ml | 50 μg/ml | 100 μg/ml | 200 μg/ml |
| R00577-EE | | | 92% | 108% |
| R00577-HE | | 4% | −2% | |
| R00577-EA | | 27% | 46% | |
| R00577-Bu | | 110% | 129% | |
| R00577-WA | | −7% | −12% | |
| Momordin lc | 84% | | 83% | |

The BuOH fraction (300 mg) was injected into a preparative C18 column (21.1×250 mm) running at 10 mL/min, the gradient started with 30% Methanol/0.1% formic acid water, then increased MeOH to 100% over 45 minutes, held at 100% MeOH for additional 15 minutes. The solvent elution run generated 56 fractions, and then those fractions were combined into 20 best pools based on HPLC profile detected at 205 nm wavelength. The compound Momordin lc was isolated in the best pool RP17 (124 mg) and displayed activity in the GAG release inhibition assay illustrated in the Example 17.

### Example 3. Process Development to Enrich Momordin lc from Kochia Extract

Kochia scoparia fruit powder (100 g) was refluxed in 500 ml of 0.25 M NaOH water solution for one hour, then centrifuged at 4000 rpm to collect first basic water extract and the extraction was repeated under the same conditions once more. The basic water extract solutions were then combined and neutralized to pH 4 with 0.29 M HCl, under which, precipitation in the solution was observed. The precipitate was separated from supernatant through centrifugation at 4000 rpm, then washed with 500 mL water to remove any salty substances and centrifuged again to remove supernatant. The water washing was repeated two more times. Then 500 mL of Ethanol was added to the solid, and the EtOH-soluble portion was concentrated and dried under high vacuum to secure 11.8 g solid that contained 14% of Momordin lc.

Kochia extracts R00577-EE (171 mg), R00577-70E (262 mg) and R00577-50E (234 mg) produced according to Example 1, were partitioned between BuOH and water, collected BuOH fraction, removed the solvent with vacuum, and quantified all three BuOH fractions with quantification method as described in Example 1. The BuOH fraction generated from 50% EtOH extract showed the highest Momordin lc content (30%).

Kochia scoparia fruits powder from another collection R00782 (520 g) was divided into two flasks in equal amount, 600 ml of 50% Ethanol/water (50E) added to each flask, refluxed for 1 hour, extract collected through filtration, reflux repeated two more times. All extracts (about 3 liters) were combined, the organic solvent reduced on a rotary-evaporator to the final volume of about 600 mL, and then the volume brought to 2 liters by adding more water. The extract was partitioned with BuOH three times, about 750 mL each time. The BuOH fraction was dried with a rotary evaporator under high vacuum to yield 34 g enriched BuOH fraction. The compound Momordin lc was enriched from 4.2% in 50% ethanol extract (50E) to 24.3% in BuOH fraction.

TABLE 4
HPLC quantification results of BuOH fraction from Kochia

| | BuOH fraction | BuOH fraction | Momordin lc |
|---|---|---|---|
| Sample ID | weight (mg) | distribution | in extract |
| R00577-EE-Bu | 103.4 | 62% | 5% |
| R00577-70E-Bu | 92.8 | 36% | 23% |
| R00577-50E-Bu | 48.3 | 22% | 30% |

### Example 4. Preparation of Extracts from Kochia scoparia in Production Pilot Scale

Dried Kochia scoparia fruits (18 kg) were crushed and extracted with 5 to 10-folds volume of ethanol under 80° C. for 1 hour, the extraction process was repeated 3 times. After each extraction, the decoction was filtered, concentrated and dried under vacuum to generate 1.8 kg extract. The extraction yield was about 10% (w/w), and the extract contained 11.8% Momordin lc.

Dried Kochia scoparia fruits powder (35 kg) was extracted with 5 to 10-folds volume of 95% ethanol under 90° C. for 1 hour, then the decoction was filtered to receive first extract in solution. Added fresh solvent to the biomass and repeated the extraction process 2 more times. Combined extract in solution from three repeats, concentrated and dried under vacuum with temperate between 70-85° C. The production process from 35 kg plant powder to extract powder was repeated three times (3×35 kg) to produce three batches of extracts with extraction yielded between 13-15%. The dried extract was ground and blended with 25% maltodextrin, then sieved to pass 80 mess to produce a fine powder extract with final production yield of 18%. The three batches of extract contained 8.4%, 9.1% and 8.6% Momordin lc, respectively.

### Example 5. Preparation and HPLC Quantification of Extracts from Piper Nigrum Fruit

Piper nigrum fruit powder (314 g) was divided into two flasks in equal amount, 600 mL organic solvent 50% Methanol in dichloromethane added to each flask, refluxed for 1 hour, extract collected through filtration, reflux repeated two more times under same conditions. All extract solutions were combined, the solvent removed on a rotary-evaporator, and the extract dried under high vacuum to yield 31 g organic extract (OE). This OE extract contained 33.7% Piperine by HPLC.

Similar results were obtained using the same procedure, but with the organic solvent being replaced with methanol or ethanol to provide a methanol extract (ME) or ethanol extract (EE), Ethanol:H2O (7:3) extracts, Ethanol:H2O (1:1) extracts, Ethanol:H2O (3:7) extracts and water extracts respectively.

The target component, Piperine, in the Piper organic extracts was quantified with a Luna C18 reversed-phase column (Phenomenex, 10 μm, 250 mm×4.6 mm) in a Hitachi HPLC system at 254 nm. The column was eluted with a binary gradient of water (mobile phase A) and Methanol (mobile phase B) at 1 mL/min flow rate and 35° C. column temperature. Reference Standard Piperine (from Sigma) was utilized as the quantification standard.

TABLE 5
Gradient Table of HPLC Analytical Method for Piperine

| Time (min) | Mobile phase A | Mobile phase B |
|---|---|---|
| 0.0 | 40 | 60 |
| 20 | 0 | 100 |
| 23 | 0 | 100 |
| 23.1 | 40 | 60 |
| 30 | 40 | 60 |

### Example 6. Preparation of Extracts from Piper nigrum in Production Pilot Scale

Dried Piper nigrum fruits (10 kg) were crushed and extracted with 5 to 10-folds volume of 70% Ethanol/water under 80° C. for 3 hours, filtered to collect extract, extracted again under the same conditions for 2 hours. Extracts from both extractions were combined, the solvent removed with rotary-evaporator and the sample dried under vacuum to generate 100 g 70% ethanol extract (70E). The extract contained 41.9% Piperine by HPLC analysis.

Dried Piper nigrum fruits were crushed and extracted with 90% ethanol in water under 80° C. The solution was concentrated under vacuum until the volume was reduced to less than 20% and sat at room temperature for precipitation. The solids were collected and recrystallized in ethanol and water solution. The standardized 15:1 Piper nigrum extract contains no less than 30% Piperine.

### Example 7. Fractionation and Purification of Piperine Extract from Piper nigrum

The organic extract (10.9 g) of dried Piper nigrum fruits obtained as described in Example 5 was subjected to silica gel column fractionation to pursue GAG releasing inhibition activity. The OE extract was divided and loaded separately onto two pre-packed Biotage flash columns (120 g silica, particle size 32-60 μm, 4 cm×19 cm), and then eluted with Hexane, EtOAc and Methanol (as the mobile phase) at a flow rate of 20 mL/minutes. The gradients started with 100% Hexane for 5 minutes, then increased EtOAc from 0% to 100% over the duration of 25 minutes, and held at 100% EtOAc for additional five minutes, then increasing MeOH from 0% to 50% MeOH/EtOAc over next period of 15 minutes, finally changed the elution solution to 100% MeOH and eluted the column for another 16 minutes. The total run time was 66 minutes and 88 fractions were generated for each column fractionation. The fractions were analyzed by silica gel thin layer chromatography (TLC) and pooled together to generate eight best pools NP1 to NP8. The GAG releasing inhibition assay (Example 17) confirmed the highest activity was in best pool 4, and it contained 77% of Piperine by HPLC analysis.

TABLE 6
Inhibition of GAG release of Piper extract and fractions

| | Inhibition of GAG release | | | |
|---|---|---|---|---|
| Sample ID | 25 μg/mL | 50 μg/mL | 100 μg/mL | 200 μg/mL |
| OE extract | | 54% | | 92% |
| NP 1 | 22% | | 68% | |
| NP 2 | 12% | | 16% | |
| NP 3 | 41% | | 78% | |
| NP 4 | 33% | | 91% | |
| NP 5 | 38% | | 76% | |
| NP 6 | | | 23% | 63% |
| NP 7 | 21% | | 52% | |
| NP 8 | 6% | | 10% | |
| Piperine | | 98% | | |

### Example 8. Preparation and HPLC Quantification of Extracts from Alpinia Rhizome

The dry Alpinia rhizomes were ground into powder. 20 grams of Alpinia rhizome powder was mixed with enough Diatomaceous earth to fill up a 100 mL extraction cell, and extracted with 100% Ethanol (EE) by using ASE 350 Extractor (Extraction condition: Heat=5 minutes, Static=5 minutes, Flush=80 volume, Purge=900 seconds, Cycles=3, Pressure=1500 psi, Temperature=60° C.). After extraction, the solution was concentrated with an evaporator at 50° C. to produce a solid extract.

Similar results were obtained using the same procedure, but with the organic solvent being replaced with methanol or ethanol to provide a methanol extract (ME) or Ethanol:H2O (7:3) extracts, Ethanol:H2O (1:1) extracts, Ethanol:H2O (3:7) extracts and water extracts respectively.

The target component l-acetoxychavicol acetate in extracts were quantified with a Luna C18 reversed-phase column (Phenomenex, 10 μm, 250 mm×4.6 mm) in a Hitachi HPLC system at 254 nm.

TABLE 7
Gradient Table of HPLC Analytical Method for 1′-
acetoxychavicol acetate

| Time (min) | Mobile phase A | Mobile phase B |
|---|---|---|
| 0.0 | 70 | 30 |
| 20 | 0 | 80 |
| 20.1 | 0 | 100 |
| 23 | 0 | 100 |
| 23.1 | 70 | 30 |
| 30 | 70 | 30 |

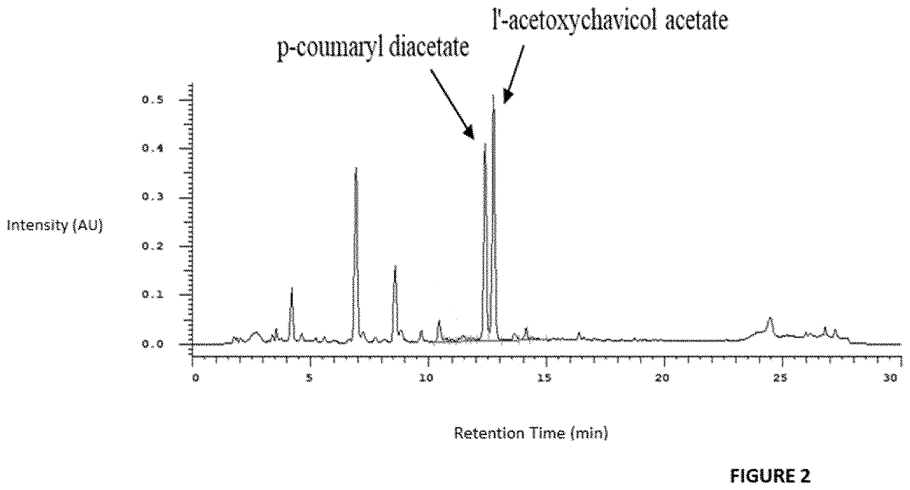

The column was eluted with a binary gradient of water (mobile phase A) and Acetonitrile (mobile phase B) at 1 ml/min flow rate and 35° C. column temperature. Reference Standard 1′-acetoxychavicol acetate purchased from LKT lab contained both l′-acetoxychavicol acetate and p-coumary diacetate peaks with chromatogram purity of 62% and 24%, respectively, was utilized as the quantification standard. FIG. 2 shows a HPLC chromatogram of Alpinia ethanol extract at 254 nm.

Alpinia plants were collected from India, China and Thailand from different geological locations and different species. The raw material powders were extracted with EtOH as described above. The yield for EtOH extraction and HPLC quantification of l′-acetoxychavicol acetate (Marker 1) and p-coumary diacetate (Marker 2) are listed in the table below.

TABLE 8
Extraction and HPLC quantification results of Alpinia extract

| | | | Marker | Marker | |
|---|---|---|---|---|---|
| | | Extraction | 1 in | 2 in | Plant |
| Sample ID | Latin Name | Yield | Extract | Extract | Origin |
| R00602-EE | Alpinia | 5% | 1.6% | 1.95% | China |
| | officinarum | | | | |
| R00778-EE | Alpinia galanga | 4% | 20% | 0.46% | India |
| R00784-EE | Alpinia galanga | 3% | 38% | 0.8% | India |
| R00787-EE | Alpinia galanga | 7% | 17% | 1.1% | India |
| R00958-EE | Alpinia galanga | 11% | 22% | 0.39% | India |
| R00959-EE | Alpinia galanga | 5% | 37% | 0.8% | India |
| R00960-EE | Alpinia | 12% | 0 | 0 | China |
| | officinarum | | | | |
| L0572-EE | Alpinia | 6% | 31% | 1.6% | China |
| | officinarum | | | | |
| L0666-EE | Alpinia galanga | 15% | 0 | 0 | India |
| L0717-EE | Alpinia galanga | 5% | 24% | 1.11% | India |
| L0718-EE | Alpinia galanga | 7% | 22% | 0.44% | India |
| L0719-EE | Alpinia galanga | 4% | 21% | 0.39% | India |
| L0720-EE | Alpinia galanga | 6% | 22% | 1.01% | India |
| P05797-EE | Alpinia galanga | 16% | 17% | 0.9% | Thailand |

### Example 9. Isolation and Purification of Active Compounds from Extract of Alpinia Rhizome

Alpinia galangal rhizome dry powder (170 g) was placed in a flask, 600 ml Ethanol was added to reflux for 1 hour, extract collected through filtration, reflux repeated two more times. All extract solutions were combined, the solvent removed on a rotary-evaporator, and the extract dried under high vacuum to yield 27 g Ethanol extract (P05797-EE). This ethanol extract contained 17% l′-acetoxychavicol acetate by HPLC analysis.

Alpinia extract P05797-EE (12 g) was partitioned between organic solvent (100 ml) and water (150 ml) in the order of Hexane, EtOAc and BuOH to generate Hexane fraction (4.2 g). EtOAc fraction (1.2 g), BuOH fraction (0.6 g) and water fraction (5.1 g). The GAG release inhibition activity was found in Hexane and EtOAc fractions. Combined both active fractions (5.4 g) and loaded onto a pre-packed Biotage flash columns (120 g silica, particle size 32-60 μm, 4 cm×19 cm), and then eluted with Hexane, EtOAc and Methanol (as the mobile phase) at a flow rate of 20 mL/minutes. The gradients started with 95% Hexane/EtOAc for 5 minutes, then increased EtOAC gradually from 5% to 100% over the duration of 35 minutes, then held at 100% EtOAc for additional 5 minutes, before increasing MeOH from 0-100% over next period of 15 minutes, finally held at 100% MeOH for another 16 minutes. The total run time was 66 minutes and 88 fractions were generated. The fractions were analyzed by silica gel thin layer chromatography (TLC) and pooled together to generate 11 best pools. The best pool NP3 and best pool NP4 contained most of the weight with potent GAG release inhibition activity.

The silica gel column best pool NP3 (200 mg) was fractionated on a preparative C18 column (21.1 mm×250 mm) with a linear gradient of 40% Methanol/water to 100% Methanol over 45 minutes at a flow rate of 10 mL/minute to generate 45 fractions, and then combined into 12 best pools based on HPLC profile at 254 nm. The best pool RP3 contained the first target compound l′-acetoxychavicol acetate (131.4 mg), and the GAG release inhibition activity (Example 17) was confirmed.

TABLE 9
Inhibition of GAG release of Alpinia extract and fractions

| | Inhibition of GAG release | | |
|---|---|---|---|
| Sample ID | 25 μg/mL | 100 μg/mL | 200 μg/mL |
| P05797-EE | | 110% | 118% |
| P05797-HE | 79% | 117% | |
| P05797-EA | 43% | 88% | |
| P05797-Bu | 6% | −2.8% | |
| P05797-WA | 8% | −2.3% | |
| NP3 | 88% | 139% | |
| NP4 | 128% | 132% | |
| 1′-acetoxychavicol acetate | 99% | 109% | |
| p-coumaryl diacetate | 111% | 138% | |

The silica gel column best pool NP4 (210 mg) was fractionated on a preparative C18 column (21.1 mm×250 mm) with a linear gradient of 30% acetonitrile/water to 80% acetonitrile over 42 minutes at a flow rate of 10 mL/minute to generate 19 fractions, and then combined into 6 best pools based on HPLC profile at 254 nm. The best pool RP3 contained the second target compound p-coumaryl diacetate (4.3 mg), and the GAG activity was confirmed.

### Example 10. Preparation of EtOH Extracts from Alpinia Rhizome in Production Scale

Dried Alpinia galanga rhizome (40 kg) were crushed and extracted with 5 to 10-folds volume of Ethanol under 80° C. for 3 hours, filtered to collect extract, extracted again under the same conditions for 2 hours. Extract solution were combined from both extractions, the solvent removed with rotary-evaporator and the sample dried under vacuum to generate 2 kg ethanol extract (EE). The extract contained about 20% 1′-acetoxychavicol acetate quantified by HPLC.

Dried Alpinia galanga rhizome were pulverized and extracted with 95% ethanol. After vacuum concentration and drying, the solid extract was crushed with addition of maltodextrin to produce an extract with 6:1 ratio of rhizome:extract. This standardized Alpinia extract contains 4%-8% compound 1′-acetoxychavicol acetate.

### Example 11. Preparation of Supercritical CO₂ Fluid Extract from Alpinia Rhizome

Alpinia galangal powder (45 g) was placed into a 100 ml stainless steel vessel and pressurized with liquid CO₂, heated to extraction temperature of 50° C. and then pressurized to extraction pressure of 640 bar before beginning the dynamic flow of supercritical CO₂. The supercritical CO₂ containing extract was depressurized into a collection vial. After 75 min, the soluble components extraction was completed and produced 1.23 g extract with yield of 2.96% (W/W) and 56.7% galangal acetate. After the completion of CO₂ extraction 5%/W/W Ethanol was added to the supercritical CO₂ and the extraction of the same sample was continued at the same conditions of temperature and pressure to produce 0.15 g extract containing 4.7% galangal acetate.

Another extraction followed the previous extract protocol but was conducted with supercritical CO₂/EtOH 5% W/W from the start of the extraction until completion of the experiment (300 min) to generate 1.18 g extraction with yield of 2.58% and 47.3% galangal acetate.

### Example 12. HPLC Quantification of Alpinia Extracts from Different Sources

Alpinia extracts were obtained from different geological location and vendors in China and India, then 1-acetoxychavicol acetate was quantified with HPLC method described in Example 8. The HPLC quantification results are shown as table below.

TABLE 10
HPLC quantification results of Alpinia extract

| | l′-acetoxychavicol | p-coumary diacetate |
|---|---|---|
| Sample ID | acetate in extract | in extract |
| L0660 | 0 | 0.04% |
| L0662 | 0 | 0 |
| L0667 | 1% | 0.12% |
| L0679 | 4% | 0.51% |
| L0680 | 0% | 0 |
| L0702 | 4% | 0.75% |
| L0703 | 4% | 0.75% |
| L0704 | 4% | 0.72% |
| L0705 | 10% | 0.43% |
| L0721 | 1% | 0.03% |
| L0722 | 1% | 0.03% |
| L0729 | 8% | 0.40% |
| L0730 | 9% | 0.41% |
| L0731 | 8% | 0.41% |

### Example 13. Preparation of 50% Extract from Magnolia officinalis

Dried Magnolia officinalis barks were crushed and extracted with supercritical CO₂, followed by concentration and vacuum drying. The dried extract was blended with 30% Maltodextrin to produce a powdery of 10:1 extract. The standardized extract contains no less than 50% of total amount of Magnolol and Honokiol combined.

The GAG activity in 50% Magnolia extract, 30% extract, pure Magnolol and Honokiol were confirmed, the results listed as table below:

TABLE 11
Inhibition of GAG release of Magnolia extracts and compounds

| | Inhibition of GAG release | | | |
|---|---|---|---|---|
| Sample ID | 10 μg/ml | 25 μg/mL | 50 μg/mL | 100 μg/mL |
| 50% Magnolia extract | | 97% | | 114% |
| 30% Magnolia extract | | 52% | | 107% |
| Magnolol | 97% | | 97% | |
| Honokiol | 104% | | 119% | |

### Example 14. Preparation of Alpinia:Magnolia:Kochia (AMK) Composition

Ethanol extract of Kochia seeds (R00835-EE, 360 g) was milled into fine powder in a food blender, then Magnolia bark extract powder (L0555, 480 g) was added to the same blender and blended to ensure the powder in uniformity. Afterward, the blended Kochia and Magnolia extract powder was transferred into a deep stainless-steel pan ready to mix with Alpinia extract. The oily Alpinia extract (R00829-EE, 240 g) was weighed out in a beaker, and sonicated in 400 ml MeOH for 1 hour, then the top liquid was transferred into the stainless-steel pan while stirred to mix with Kochia and Alpinia blend. Some remaining solid in beaker was sonicated in more MeOH for an additional 3 times with 100 ml each, and each time, the top liquid was transferred to the pan to mix, thus all Alpinia extract was transferred in MeOH to the stainless steel pan to generate a blend of Alpinia:Magnolia:Kochia (AMK) in a weight ratio of 2:4:3. The mixed slurry was dried under vacuum in an oven at 45° C. for one week, and then ground in a bench-top herb grinder to secure 1042 g find powder. Based on quantification results for each ingredient and blending ratio, this AMK composition contained about 4% l-acetoxychavicol acetate, 22% Magnolol/Honokiol, and 4% Momordin lc.

Another AMK composition in a weight ratio of 5:4:4 was prepared by blending all components in powder form as described here. Alpinia extract (L0795, 30 g), Magnolia bark extract (L0789, 24 g) and Kochia seeds extract (L0798A, 24 g) were weighed out separately and placed into a food blender to mix to secure a consistent powder. Based on quantification results of each ingredient and blending ratio, this AMK 5:4:4 composition contained about 3% 1′-acetoxychavicol acetate, 18% Magnolol/Honokiol, and 3% Mormodin lc.

Individual extracts of Alpinia, and/or Pepper, and/or Magnolia and/or Kochia could be combined to a composition with 3 individual extracts at different ratios including 1:1:1, 2:1:1, 3:1:1, 4:1:1, 5:1:1, 1:2:1, 1:3:1, 1:4:1, 1:5:1, 1:1:2, 1:1:3, 1:1:4, 1:1:5, 1:2:3, 1:2:4, 1:2:5, 1:2:6, 1:2:6, 1:2:8, 1:2:9 or 1:2:10 etc. by weight, respectively.

### Example 15. Preparation of Alpinia:Piper/Pepper (AP) Composition

Extraction methods and quantification of bioactives from Alpinia rhizomes and Pepper fruits have been disclosed in Examples 10, 11, 12 and 6, 7, 8; respectively. The Alpinia:Pepper (AP) composition was prepared by weighing Alpinia and pepper extracts in 1:2 ratio by weight into a vial and added proper solution to sonicate and vortex for homogeneity before each assay. The composition contained about 7% l′-acetoxychavicol acetate and 20% Piperine.

Individual extracts of Alpinia, and Pepper could be combined to a composition at different ratios in a range from about 0.5:5 to about 5:0.5, including 0.5:1, 0.5:2, 0.5:3, 0.5:4, 0.5:5, 1:1, 1:2, 1:3, 1:4, 1:5, 2:1, 2:2, 2:3, 2:4, 2:5, 3:1, 3:2, 3:3, 3:4, 3:5, 4:1, 4:2, 4:3, 4:4, 4:5, 5:1, 5:2, 5:3, 5:4, 5:5, 1:0.5, 2:0.5, 3:0.5, 4:0.5, or 5:0.5 by weight respectively.

### Example 16. Preparation of Extracts from Alpinia, Piper/Pepper, and Magnolia for Topical Applications

Many herbs have been used as anti-inflammation and pain control with application orally or topically as alternative medicine. Their pain relief and anti-inflammatory properties are associated with a broad range of types of bioactive compounds with potential targeting through different mechanisms and pathways. We are looking for analgesics after preparation and topical application which could penetrate the skin and function where they are needed, including alkaloids from Piper nigrum, bisphenolic lignans of Magnolia officinalis and phenylpropanoid Galanga acetate from Alpinia galanga. Those actives represent different types of chemical constituents and could deliver pharmacological effects through different mechanisms.

Alpinia, Pepper and Magnolia extracts were prepared at a concentration of 50 mg/mL in a combination of DMSO, Propylene glycol, Aloe vera gel (1:2:1) or a combination of DMSO, Propylene glycol and MCT oil (1:1:2) depending on the property and solubility of each material. Aloe vera gel served a penetration enhancer to improve the skin penetration during topical administration.

### Example 17. Procedures for Testing Inhibition of Glycosaminoglycan (GAG) Release by Individual Extracts, Fractions and Compounds of Alpinia, Piper/Pepper, Magnolia, and Kochia Plants

Cartilage tissue is primarily composed of extracellular matrix secreted by chondrocytes. The individual components of the tissue include collagen II fibers, hyaluronic acid and proteoglycans, which are composed of a glycosaminoglycan (GAG), such as chondroitin sulfate or keratin sulfate, bonded to a protein core. Enzymatic breakdown of cartilage tissue leads to free molecules of these components in the extracellular matrix and resorption by the body.

### Cartilage Explant Cultures

Articular cartilage from hock joints of rabbits (2.5 kg body weight) was removed immediately after each animal was sacrificed. The articular cartilage explants were obtained by following the method described by Sandy et al, 1986. Briefly, after the articular surfaces were exposed surgically under sterile conditions, approximately 200-220 mg articular surfaces per joint were dissected and submerged into complete medium (DMEM, supplemented with heat inactivated 5% FBS; penicillin 100 U/ml; streptomycin 100 μg/ml). They were then rinsed several times with the complete medium and incubated for 2 days at 37° C. in a humidified 5% CO₂/95% air incubator for stabilization. The complete medium was replaced with a basal medium (DMEM, supplemented with heat-inactivated 1% FBS, 10 mM HEPES, and penicillin 100 U/ml streptomycin 100 μg/ml). Approximately 30 mg cartilage pieces (2×3×0.35 mm/piece) were placed in 48-well plates and treated with given concentrations of test agents. After pretreatment for 1 h, 5 ng/ml of rhIL-1α was added to the culture medium and further incubated at 37° C. in a humidified 5% CO₂/95% air incubator. The culture medium was collected 24 h later and stored at −80° C. until assay.

### Glycosaminoglycan Measurements

The amount of sulphated GAGs in the medium at the end of the reaction, reflecting the amount of articular cartilage degradation, was determined through 1,9-dimethy-methylene blue method using commercially available kit (the Blyscan proteoglycan and glycosaminoglycan assay) according to the instructions of the manufacturer. Diclofenac was utilized as a positive control at 300 μg/ml.

### Example 18. Inhibition of Glycosaminoglycan (GAG) Release by Individual Extracts of Alpinia, Piper/Pepper, Magnolia, and Kochia Plants

Dose curves of Alpinia, Pepper, Magnolia, and Kochia plant extracts were tested on the ex vivo glycosaminoglycan (GAG) release assay as illustrated in previous example to assess their cartilage protection effects. Cartilage explants were pre-treated with each extract before being exposed to IL-1α, which caused degradation and the release of GAGs from the cartilage matrix. The ability of each extract to reduce GAG release was found to be dose responsive, with IC₅₀ values as indicated in the table below. Magnolia extract caused the greatest protective effect, with an IC₅₀ of 17.9 μg/mL. Pepper and Kochia extracts showed roughly the same amount of protection from cartilage degradation, with IC₅₀ values of 40.8 μg/mL and 42.1 μg/mL, respectively. Of the four extracts tested, Alpinia showed the least protective effect, with an IC₅₀ value of 71.6 μg/mL. All four of the extracts tested protected cartilage from degradation, as demonstrated by this assay.

TABLE 12
GAG release IC₅₀ values for individual extracts of
Alpinia, Pepper, Magnolia, and Kochia plants

| Extracted Plant | GAG release IC₅₀ (μg/mL) |
|---|---|
| Alpinia | 71.6 |
| Pepper | 40.8 |
| Magnolia | 17.9 |
| Kochia | 42.1 |

The inhibition of GAG release exhibited by the four extracts tested indicates that they inhibit the degradation of cartilage, meaning that they inhibit cartilage catabolism. We further explored this function by testing the direct inhibition of Matrix Metalloproteinases (MMPs) by the extracts, and by testing for their effects on transcription of catabolic effectors.

### Example 19. Procedures for Testing Inhibition of Matrix Metalloproteinases (MMPs) by Individual Extracts of Alpinia, Piper/Pepper, Magnolia, and Kochia Plants

Individual extracts of Alpinia, Pepper, Magnolia, and Kochia plants were incubated at 100 μg/mL with Matrix Metalloproteinase-9 (MMP-9) or Matrix Metalloproteinase-13 (MMP-13) in 50 mM MOPS, pH 7.2, 10 mM CaCl₂), 10 μM ZnCl₂, 1% DMSO, 0.05% Brij 35 with 4.0 μM Mca-Pro-Leu-Gly-Leu-Dpa-Ala-Arg-NH2 (hereafter referred to as “substrate”). The substrate exhibits fluorescence and is cleaved by both MMP enzymes, reducing fluorescent output. The substrate, each MMP enzyme, and each extract were incubated for two hours at 37° C., and the amount of substrate was quantified spectrofluorimetrically. Percent inhibition of each MMP enzyme was calculated for each individual extract as compared to the vehicle control. TIMP-2 was used as a positive control for the inhibition of MMPs.

### Example 20. Inhibition of Matrix Metalloproteinases (MMPs) by Individual Extracts of Alpinia, Piper/Pepper, Magnolia, and Kochia Plants

MMP enzymes are expressed by chondrocytes in cartilage tissue and play important roles in the degradation of cartilage, as they are catabolic biomarkers for OA disease progression. Secretion of MMP-13 by the chondrocytes takes place after cytokine release and increased inflammatory signaling and leads to degradation of the cartilage through the collagenase activity of the enzyme. MMP-9 is a gelatinase enzyme that further breaks down partially digested collagen. (Gepstein et al, 2002). Direct inhibition of either enzyme, but especially MMP-13 may reduce their activities and lead to a decline in the catabolic pathways responsible for cartilage breakdown.

100 μg/mL of each individual extract of Alpinia, Pepper, Magnolia, and Kochia plants was tested for inhibition of MMP-9 and MMP-13 by measuring the amount remaining of a fluorescent substrate after incubation with the test extract. Percent inhibition of each MMP by each extract is shown in the table below. Piper/Pepper and Magnolia extracts inhibited MMP-13 by 70% and 68%, respectively, and MMP-9 by 44% and 36%, respectively. Alpinia extract inhibited MMP-13 by 38%, but did not significantly inhibit MMP-9, as percent inhibition was only 3%. Kochia extract moderately inhibited both enzymes with 10% inhibition of MMP-13 and 3% inhibition of MMP-9. Pepper, Magnolia, and Alpinia extracts reduce the activity of MMPs through direct binding and inhibiting of the MMP enzymes. This has important implications for the effects of these individual extracts on the catabolic pathways associated with cartilage degradation.

TABLE 13
Inhibition of MMPS by individual extracts of Alpinia,
Pepper, Magnolia, and Kochia

| Extract (100 μg/mL) | MMP-13% inhibition | MMP-9% inhibition |
|---|---|---|
| Alpinia | 38 | 3 |
| Pepper | 70 | 44 |
| Magnolia | 68 | 36 |
| Kochia | 10 | 3 |

### Example 21. Molecular Biological Procedures for Human Chondrocyte Treatment with IL-1 and Quantification of Anabolic and Catabolic Gene Expression

### Preparation of Cells

Human chondrocytes (ScienCell, catalog #4650) were thawed and seeded in a T75 Falcon® Tissue Culture Flask (VWR, catalog #BD353136) using Chondrocyte Growth Medium (Sigma, catalog #411-500). The cells were incubated at 37° C. and 5% CO₂ for 24 hours, at which point the media was replaced with fresh, 37° C. Chondrocyte Growth Medium. Incubation at 37° C./5% CO₂ continued for an additional 48 hours, or until the chondrocytes became ˜90% confluent. The media was aspirated, and cells were rinsed once with 2-4 mL PBS (VWR, catalog #VWRL0119-0500). 2 mL trypsin-EDTA (Sigma, Catalog #T3924-100ML) was added to the flask and left to sit for ˜3-5 min or until most of the cells were detached. 8 mL of trypsin inhibitor (Sigma, Catalog #T6414-100ML) was added to the flask to bring the total volume to 10 mL. The cell solution was transferred to a 15 mL conical tube and centrifuged for 5 min at 1000 rpm. The supernatant was aspirated, and the chondrocytes were resuspended in 1 mL Chondrocyte Growth Medium. To count the cells, a 10 μL aliquot of resuspended cells was added to 90 μL trypan blue (VWR, Catalog #12002-038). 10 μL of this solution was added to a hemocytometer. The cells were then seeded into 24 and 96-well plates (VWR, Catalog #62406-159, 62406-08) using Chondrocyte Growth Medium from Sigma at a density of ˜13,200 cells per cm² (25,000 cells/well for 24-well plates and 4,200 cells/well for 96-well plates). Chondrocytes were incubated for 24 hours at 37° C./5% CO₂. The media was aspirated, and the chondrocytes were serum-starved using Chondrocyte Medium, Basal (ScienCell, Catalog #4651-b). Cells were incubated for 24 hours at 37° C./5% CO₂.

Pretreatment with IL-1β

10 ng/mL IL-1β pretreatment was prepared using IL-1β (Sigma, Catalog #SRP3083) and ScienCell's Basal Chondrocyte Medium. Old media was aspirated and replaced with 500 μL pretreatment solution for the 24-well plates, and 100 μL for the 96-well plates. A few replicates were used as a control and were not pretreated with 10 ng/mL IL-1β. Instead, fresh Basal Chondrocyte Medium was added to the cells. The cells were incubated for another 24 hours at 37° C./5% CO₂.

### Treatment of Cells

Treatments were prepared using plant extract stored at a concentration of 1M or 50 mg/mL in DMSO and ScienCell's Basal Chondrocyte Medium. 50 μg/mL Piascledine300 and 100 ng/mL BMP-2 protein (Sigma, catalog #SRP6155-10UG) were used as positive controls. All treatments were filtered using VWR's Vacuum Filtration Unit (VWR, catalog #10040-460), and brought to an IL-1β concentration of 10 ng/mL (with the exception of the untreated control). The vehicle treatment consisted only of Basal Chondrocyte Medium and 10 ng/mL IL-1β. Old media was aspirated from each well and replaced with 500 μL treatment for the 24-well plates, and 100 μL for the 96-well plates. All treatments were applied in triplicate. The cells were incubated at 37° C./5% CO₂ for 72 hours.

### RNA Extraction and RT-PCR

After 72-hour treatment exposure, media was aspirated from the 24-well plates and the cells were lysed using Qiagen's RNeasy kit (Qiagen, catalog #74104) and QIAshredder kit (Qiagen, catalog #79656). 350 μL RLT buffer with 1% β-Mercaptoethanol was initially added to each well, then the lysate mixture was transferred to a QIAshredder column for completion of the lysing step. The remainder of the RNA extraction procedure was completed according to the manufacturer's instructions. RT-PCR was performed using SuperScript IV First-Strand Synthesis System (Life Technologies, catalog #18091200) according to the manufacturer's instructions.

cDNA Quantification and Dilution

cDNA was quantified using the Qubit ssDNA Assay Kit (Life Technologies, catalog #Q10212) according to the manufacturer's instructions. Each cDNA sample was diluted to 2.5 ng/mL with dH₂O.

qPCR
The following primers (Life Technologies, catalog #A15612) were diluted to 8 μM in dH₂O:

Col2A1

| | |
|---|---|
| F: AGACTTGCGTCTACCCCAATC | R: GCAGGCGTAGGAAGGTCATC |

ACAN

| | |
|---|---|
| F: CACGATGCCTTTCACCACGAC | R: TGCGGGTCAACAGTGCCTATC |

Sox-9

| | |
|---|---|
| F: GCTCTGGAGACTTCTGAACGA | R: CCGTTCTTCACCGACTTCCT |

TGFb1

| | |
|---|---|
| F: GCAAGTGGACATCAACGGGT | R: TCCGTGGAGCTGAAGCAATA |

MMP-3

| | |
|---|---|
| F: TGGACAAAGGATACAACAGGGAC | R: ATCTTGAGACAGGCGGAACC |

MMP-13

| | |
|---|---|
| F: AACGCCAGACAAATGTGACCC | R: TCCGCATCAACCTGCTGAGG |

ADAMTS4

| | |
|---|---|
| F: GCAACGTCAAGGCTCCTCTT | R: CTCCACAAATCTACTCAGTG |
| | AAGCA |

GAPDH

| | |
|---|---|
| F: CAAGGCTGAGAACGGGAAGC | R: AGGGGGCAGAGATGATGACC |

Each qPCR reaction consisted of 400 nM forward primer, 400 nM reverse primer, 1 ng/μL cDNA, and was brought to a total volume of 24 μL with PowerUp™ SYBR™ Green Master Mix (Applied Biosystems, Catalog #A25742). 10 μL of each reaction was plated in duplicate on a 96-well reaction plate (Applied Biosystems, catalog #4366932) and run on the Applied Biosystems StepOnePlus Real-Time PCR System according to the following cycling conditions: 50° C./2 minutes; 95° C./2 minutes; 40× [95° C./15 seconds-60° C./1 minute].

### Cell Viability

After 72-hour treatment exposure, 20 μL of CellTiter 96@ AQueous One Solution Cell Proliferation Assay (Promega, catalog #G3580) was added to each well in the 96-well plate. 100 μL Basal Chondrocyte Medium was used as a blank. The plate was gently tapped to mix solution, and the chondrocytes were incubated at 37° C./5% CO₂ for 30 minutes. After incubation, the absorbance of each well was measured at 492 nm.

### Example 22. Anabolic and Catabolic Gene Expression in Human Chondrocytes Treated with the Extracts of Alpinia, Magnolia, Kochia, Piper/Pepper, and a Combination of Alpinia and Pepper

25 μg/mL Magnolia extract resulted in significant decreased expression of catabolic MMP-3 and MMP-13, with limited and not significant changes in anabolic gene expression. These data indicate that Magnolia extract works to combat cell degradation by interfering with inflated catabolic gene expression in the presence of extracellular IL-1β. Similar to Magnolia extract, at 10 μg/mL Alpinia extract significantly reduced MMP-13 gene expression while significantly up regulating SOX-9, ACAN and COL2A1. Enriched Kochia extract significantly upregulated anabolic ACAN, Sox-9, and TGFβ1 gene expression, while having a lesser effect on downregulation of the catabolic markers. Pepper extract had a similar effect, causing upregulation of COL2A1, ACAN, SOX-9, and TGFβ1, while not significantly affecting catabolic markers. The combination of Alpinia:Pepper showed marked synergy of MMP-13 inhibition, while also exhibiting a decrease in ADAMTS4 and maintaining an increase in TGFβ1.

TABLE 14
Fold changes of the expression of anabolic and catabolic
genes in human chondrocytes incubated with
the extracts of Alpinia, Magnolia and Kochia

| | Fold expression changes relative to vehicle | | | | |
|---|---|---|---|---|---|
| | Kochia | Magnolia | Alpinia | Pepper | Alpinia: |
| | extract | extract | extract | extract | Pepper |
| | (5 | (25 | (10 | (25 | (24 |
| Genes | μg/mL) | μg/mL) | μg/mL) | μg/mL) | μg/mL) |
| COL2A1 | −0.36 | −0.55* | −0.4 | 0.593* | −0.12 |
| ACAN | 0.46* | −0.24 | 0.41* | 0.24* | −0.05 |
| SOX-9 | 1.49** | −0.44 | 1.29* | 0.57** | 0.35 |
| TGF-β1 | 1.98*** | −0.59 | 2.69*** | 0.26* | 1.00* |
| MMP-3 | −8,979 | −131,445** | −3,040 | 4,452 | −895 |
| MMP-13 | −193 | −699* | −212* | −13 | −1,924** |
| ADAMTS4 | −16¹ | −5.22 | −16¹ | −0.23 | −1.30* |

C_{T} values from the qPCR were used to calculate ΔΔC_{T}, which was normalized to the untreated control. ΔΔC_{T} values were used to calculate the fold expression change of each gene. The values in the table indicate the difference in fold expression change between each treatment and the vehicle control.
*P ≤ 0.05;
**P ≤ 0.005;
***P ≤ 0.0005;
¹Outlier data.

In summary, Magnolia extract can contribute downregulation of catabolic homeostasis, both Kochia and Pepper extracts can upregulate gene expression of anabolic pathways of chondrocytes, and Alpinia alone and in combination with Pepper extract can demonstrate both activities.

### Example 23. Molecular Biological Procedures for Rat Chondrocyte Treatment with IL-1 and Quantification of TGF-β1 Gene Expression

### Preparation of Cells

Chondrocytes for monolayer cultures were isolated from knee cartilage of young rats and cultured as follows: Sprague Dawley rats, 3 weeks of age, were euthanized and their hind limbs were collected. Knee cartilage was cut from the subchondral bone using a sterile scalpel blade. Cartilage shavings were digested with collagenase in serum-free Dulbecco's Modified Eagle's Medium (DMEM). Once digested, the cell suspension was centrifuged to obtain a cell pellet. This pellet was resuspended in DMEM containing 10% FBS and the cells were counted. The cells were then plated on tissue culture plastic at a density of 10,000 cells/cm². The isolated chondrocytes were then amplified in monolayer in culture medium (DMEM/FCS-10% supplemented with HEPES (25 mM)) until passage 1 and frozen at −80° C. Thawed chondrocytes were used in the experiment described.

Chondrocytes were seeded at day-1 and cultured in monolayers in 12-well plates for 24 hours. Treatments (including IL-1β) began at day 0 and were performed over 3 days. The following 17 treatments or control conditions were carried out:
Untreated cells (cultured in proliferation medium)
IL-1β treated cells
IL-1β treated cells+vehicle
IL-1β treated cells+BMP-2 (100 ng/mL)
IL-1β treated cells+Alpinia (25 μg/mL)
IL-1β treated cells+Pepper (25 μg/mL)
IL-1β treated cells+Magnolia (25 μg/mL)
IL-1β treated cells+Kochia (100 μg/mL)

All treatments and controls were carried out in triplicate. Kochia extract was found to be non-toxic at 100 μg/mL and was tested at that concentration. The extracts of Alpinia, Pepper, and Magnolia were tested at 25 μg/mL due to cytotoxicity. Chondrocytes were lysed and total RNA was purified using the NucleoSpinR RNA II kit (Macherey Nagel).

### Treatment of Cells

Treatments were prepared using plant extract stored at a concentration of 100 mg/mL in DMSO and diluted in culture medium. 100 ng/mL BMP-2 protein (RandD Systems, catalog #355-BM-010) was used as positive control. All treatments were brought to an IL-1β concentration of 10 ng/mL (with the exception of the untreated control). The vehicle treatment consisted only of chondrocyte medium, 0.1% DMSO and 10 ng/mL IL-1β. All treatments were applied in triplicate. The cells were incubated at 37° C./5% CO₂ for 72 hours.

### RNA Extraction and RT-PCR

Chondrocytes were lysed and total RNA was purified using the NucleoSpinR RNA II kit (Macherey Nagel). One microgram of total RNA was retro-transcribed using M-MLV RT (Life Technologies). RT-PCR was performed using SuperScript IV First-Strand Synthesis System (Life Technologies, catalog #18091200) according to the manufacturer's instructions.

qPCR
The following primers were used:

TGFb1

| | |
|---|---|
| F: CCCCTGGAAAGGGCTCAACAC | R: TCCAACCCAGGTCCTTCCTA |
| | AAGTC |

RPL19

| | |
|---|---|
| F: TGCCGGAAGAACACCTTG | R: GCAGGATCCTCATCCTTCG |

B-actin

| | |
|---|---|
| F: CCAACCGTGAAAAGATGACC | R: ACCAGAGGCATACAGGGACA |

Each qPCR reaction consisted of 5 μL iQ™ SYBR Green Supermix (Biorad, ref 1708882), 0.6 μL of forward primer (5 μM), 0.6 μL of reverse primer (5 μM), 1.8 μL H2O, and 2 μL cDNA (5 μg/μL).

### Example 24. TGF-β1 Gene Expression in Rat Chondrocytes Treated with the Extracts of Alpinia, Pepper, Magnolia and Kochia in an Independent Trial

25 μg/mL Magnolia extract and 100 μg/mL Kochia extract resulted in significant increased expression of anabolic TGF-β1, whereas Alpinia and Pepper extracts did not have a significant effect. In this study, using primary rat chondrocytes, IL-1β was added at the same time as the treatment, and there was no pre-treatment. Under these conditions, it's clear that Magnolia and Kochia extracts contribute to chondrogenesis through upregulation of TGF-β1, a regulator of chondrogenic gene expression.

TABLE 15
Fold changes of the expression of anabolic and catabolic
genes in human chondrocytes incubated with
the extracts of Kochia, Pepper, Magnolia and Alpinia

| | Fold gene expression changes relative to vehicle | | | |
|---|---|---|---|---|
| | Kochia | Pepper | Magnolia | Alpinia |
| | extract | extract | extract | extract |
| Genes | (100 μg/mL) | (25 μg/mL) | (25 μg/mL) | (25 μg/mL) |
| TGF-01 | 2.837* | 0.805 | 1.820** | 0.988 |

CT values from the qPCR were used to calculate MET, which was normalized to the untreated control. ΔΔC_{T} values were used to calculate the fold expression change of each gene. The values in the table indicate the difference in fold expression change between each treatment and the vehicle control.
*P ≤ 0.05;
**P ≤ 0.005;
***P ≤ 0.0005.

In summary, Magnolia extract can contribute downregulation of catabolic genes in human chondrocytes and upregulation of TGF-β1 in rat chondrocytes, while both Alpinia and Kochia extracts can upregulate anabolic gene expression of human chondrocytes while downregulating catabolic genes. Kochia extract also showed upregulation of TGF-β1 gene expression in rat chondrocytes, solidifying its role as an anabolic effector in chondrocyte homeostasis.

### Example 25. Animals and Housing

Rats were purchased from a USDA approved vendor. Sprague Dawley rats were purchased at the age of 8 weeks and acclimated upon arrival for a week before being assigned randomly to their respective groups. Rats (3/cage) were housed in polypropylene cages and individually identified by numbers on their tails. Each cage was covered with a wire bar lid and filtered top (Allentown, N.J., USA). Individual cages each had a cage card to indicate the project number, test article, dose level, group, and animal numbers for identification. Harlan T7087 soft cob bedding was used and changed at least twice weekly. Animals were provided with fresh water and rodent chow diet #T2018 (Harlan Teklad, 370 W, Kent, Wash., USA) ad libitum and housed in a temperature-controlled room (22.2° C.) on a 12 h light-dark cycle. All animal experiments were conducted according to the institutional guidelines congruent with the guide for the care and use of laboratory animals.

### Example 26. Principle of Osteochondral Defect (OCD) Model

Through the years, various in vivo models have been introduced for the treatment of chondral defects. Among these, the microfracture technique is one of the few methods utilized to stimulate the bone marrow in the repairing process by taking advantage of the body's own healing potential. This technique enhances the chondral resurfacing by providing a suitable environment for new tissue formation. At the time of model induction, the exposed weight bearing surface of the femur subchondral bone plate will be drilled with a precision drill bit until fat droplets and blood come out of the microfractured hole into the knee. This marrow “super clot” provides an optimal environment for the body's own marrow cells (mesenchymal stem cells) from the bone marrow to differentiate into appropriate articular cartilage-like cell lines that in turn produce the extracellular matrix which eventually matures into a stable repaired tissue.

The healing process occurs through a protracted period where post-operative management plays a critical role for a quicker and successful recovery. Natural dietary supplements for joint care with anabolic activity could in fact assist a faster recovery by enhancing the body's cartilage regeneration process.

In our lab, we developed the modified microfracture induced injury in vivo model and evaluated Alpinia:Piper/Pepper (AP) and Alpinia:Magnolia:Kochia (AMK) compositions at 200 mg/kg for their anabolic (cartilage synthesis, renewal, rebuild) activity following a daily oral administration for 6 weeks post-model induction. Piascledine (avocado/soybean unsaponifiables) at oral dosage of 200 mg/kg was used as a positive control. Piascledine (avocado/soybean unsaponifiables) is a dietary supplement promoted by the manufacturer as an OA disease modulator with catabolic and anabolic effects demonstrated in preclinical in vitro and in vivo models. It is reported to possess properties known to prevent cartilage degradation by inhibiting the release and activity of matrix metalloproteinases and by increasing tissue inhibitors of these catabolic enzymes. Its cartilage repair activity was also suggested as a result of inhibition of inflammatory cytokines (Christiansen et al., 2015; Goudarzi et al., 2018)

### Example 27. OCD Model Induction and Dosing

The study included a total of 55 rats divided into 5 groups (N=11/group). Five additional rats were utilized for drill bit size determination and model optimization. Drill bit sizes of 0.35, 0.6, 0.9, 1.35 and 2 mm were tested, and the 1.35 mm drill bit was chosen. Rats received the respective dosages through the oral route daily for 2 weeks before model induction. Groups included: G1=Normal control, G2=OCD model vehicle control, G3=Piascledine (200 mg/kg), and G4=AP (200 mg/kg) and G5=AMK (200 mg/kg).

On the pre-induction treatment start day, average body weight per group was 357.3±16.4 g. Older rats were chosen for this study to minimize the interference of spontaneous recovery. Rats were gavaged daily with freshly prepared respective material suspended at 10 ml/kg per rat for 2 weeks before induction. Samples in solution were vortexed between animals to maintain the homogeneity of test materials. Following a baseline measurement for weight (368.7±4.4 g), on the induction day, a small incision was made on the left hind knee of isoflurane-sedated rats and the subchondral bone on the weight bearing surface of the femur was exposed. A drill bit (1.35 mm) was then used to carefully induce a modified microfracture drill hole using a finger spin on the exposed surface until blood was visible as an indication for the adequate penetration of the bone marrow for all the groups except for the rats in the normal control group, which followed the same surgical procedure without the drilling. The joint capsule and the skin were sutured using a 4-0 coated vicryl absorbable suture and the animals were placed back in their cages to recover from anesthesia.

TABLE 16
Study groups of the Osteochondral defect model (OCD)

| Group | N | Dose (mg/kg) |
|---|---|---|
| Control + vehicle | 11 | 0 |
| OCD + vehicle | 11 | 0 |
| OCD + Piascledine | 11 | 200 |
| OCD + AP | 11 | 200 |
| OCD + AMK | 11 | 200 |

After induction, oral treatments were continued for 6 weeks. Rats were monitored for pain sensitivity using the incapacitance meter for weight bearing measurements at week 6. At the end of the study, serum was collected for biomarker analysis. At necropsy, the femorotibial joint was dissected out, coded in a double blind way, preserved with formalin and sent to Nationwide Histology for histopathology analysis of the affected structure. Photos of the joint tissues were taken for each rat for all groups. Histopathology analysis was carried out on blinded tissues by a third-party certified pathologist as the end point measurement for this study

### Example 28. Weight Bearing as a Measure of Pain Sensitivity in OCD Model

The incapacitance tester was used to measure the weight bearing for OCD rats treated with Alpinia:Piper/Pepper (AP) and Alpinia:Magnolia:Kochia (AMK) compositions produced in example 14 and 15 against vehicle and positive controls. In this method, rats shift their body weight to the normal (unaffected) leg to relieve the pain induced by weight to the surgical leg. In the current study, rats were expected to put more weight on the right leg. As the healing progressed, the weight distribution could have changed to reflect the homeostasis of “arthritis” in terms of the speed of healing and the tolerance of pain for each leg. Surgery was performed on the left leg of each rat in all the groups. All the rats received drilling on the left leg except the normal control (NC) group which underwent the same surgery procedure without the drilling.

TABLE 17
Weight bearing as a measure of pain sensitivity for OCD rats treated
with AMK and AP compositions against vehicle and positive controls

| | | | Weight Bearing | | | | |
|---|---|---|---|---|---|---|---|
| | | | | | | % | P-values |
| | | | | | | Improvement | vs |
| | | | Left | Right | Difference | compared | Vehicle |
| | Dose | | Leg | Leg | (R-L) | to | treated |
| Group | (mg/kg) | N | Mean ± SD | | | Vehicle | OCD |
| NC | 0 | 11 | 135.1 ± 19.5 | 148.3 ± 27.8 | 13.2 ± 33.0 | 84.0 | 0.001 |
| Vehicle | 0 | 11 | 108.5 ± 23.9 | 191.0 ± 40.6 | 82.5 ± 39.3 | — | — |
| Piascledine | 200 | 11 | 131.9 ± 19.0 | 176.8 ± 29.5 | 44.9 ± 31.5 | 45.6 | 0.038 |
| AMK | 200 | 11 | 128.8 ± 9.7 | 161.9 ± 15.7 | 33.1 ± 15.6 | 59.9 | 0.003 |
| AP | 200 | 11 | 117.1 ± 15.4 | 157.1 ± 13.5 | 40.0 ± 16.9 | 51.5 | 0.006 |

As seen in table 17, rats shifted their body weight to the contralateral paw (normal right leg without surgery) to relieve the pain. As time went by, rats progressively started to put more weight on the left leg, making the right leg carry less weight. In week 6, there was statistically significant weight distribution between the right and left legs of rats treated with Alpinia:Piper/Pepper (AP) and Alpinia:Magnolia:Kochia (AMK) compositions compared to the vehicle group, which still heavily favored their right leg. When compared to the vehicle treated group, rats treated with AMK and AP compositions showed a 59.9% and 51.5% improvement in weight bearing, respectively. Compared to the normal control rats, vehicle treated OCD rats put on 6-fold increased weight on the right leg. The positive control, Piascledine showed 45.6% improvement.

### Example 29. Histopathology Analysis for Cartilage Regeneration Markers in the OCD Model

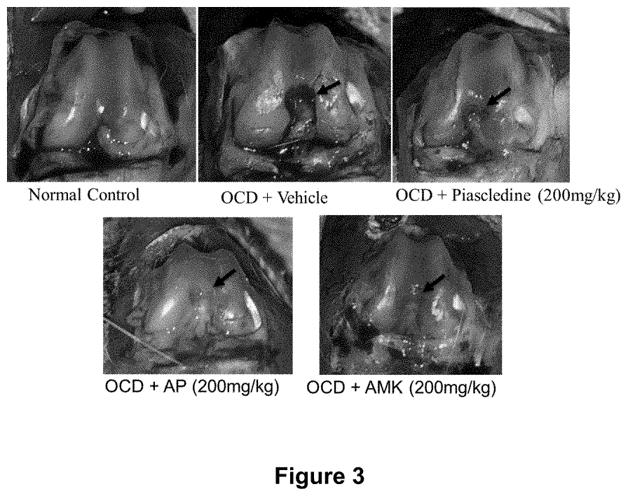

Hematoxylin and Eosin (HE) and Safranin O green staining were carried out according to Nationwide Histology's protocols. Induction of the model was confirmed by visual observation of a drill hole on the knee (FIG. 3). This was also later confirmed by the histopathology findings showing the normal appearance and scores that were given for the tissue evaluations. Each specimen underwent 3 sections for HE and an additional 3 sections for the Safranin O stain with identical orientation for each block aiming to pass through the drilled hole. It is stated that the more comprehensive quantitative histological scoring system (such as Sellers method) could yield a higher power of discrimination between different degrees of cartilage repair, resulting in enhanced sensitivity and specificity in the pathophysiological condition of articular cartilage repair. As such, we suggested to the pathologist to adapt the Sellers evaluation method for this study and tabulated the data as seen in Tables 18 and 19.

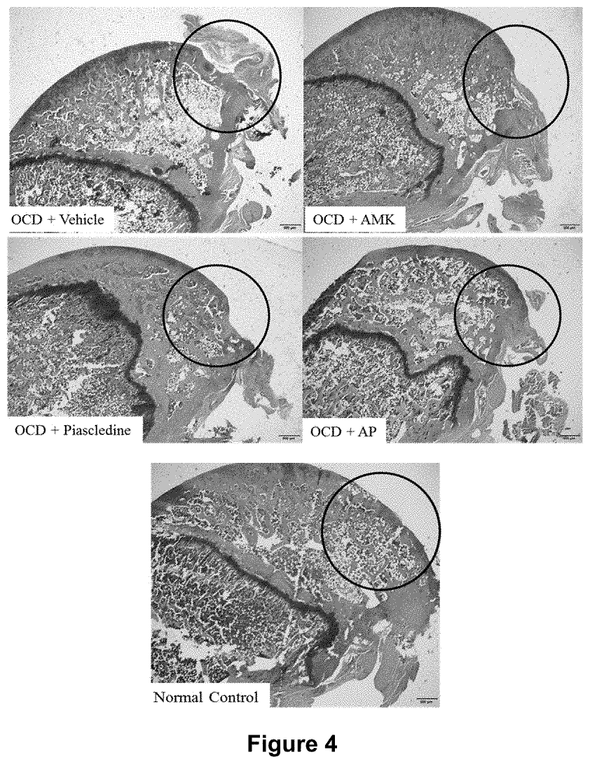

As seen in Table 19 and FIGS. 3 and 4, rats treated with AP composition showed statistically significant improvements in: 1. filling of the defect relative to the surface of the normal adjacent cartilage, 2. integration of repaired tissue with the surrounding articular cartilage, 3. matrix staining, and 4. cellular morphology compared to the vehicle treated group. These improvements were found to be 48.9%, 73.5%, 28.7% and 50.5% when compared to vehicle treated disease model rats, respectively. A strong trend of significance was also observed for the architecture of the surface and formation of tidemarks with 37.7% (p=0.07) and 32.3% (p=0.07), respectively, when compared to the vehicle group. Similarly, rats treated with AMK composition showed statistically significant improvement of: 1. integration of repair tissue with surrounding articular cartilage, 2. matrix staining with Safranin O-fast green, 3. cellular morphology, 4. architecture of the surface and formation of tidemarks compared to the vehicle treated group. These improvements were found to be 62.5%, 33.0%, 47.9%, 44.3% and 43.2% when compared to vehicle treated disease model rats, respectively. On the other hand, the positive control, Piascledine, showed statistically significant improvement in integration of repair tissue with surrounding articular cartilage (62.5% improvement vs vehicle treated OCD model) and architecture of surface (35.7% improvement vs vehicle treated OCD model) when compared to the vehicle-treated disease model. FIG. 3 shows images of a drill site of OCD rats after 6 weeks of treatment showing significant differences in healing progress from different oral treatment groups.

The histopathological results clearly demonstrated the anabolic changes to damaged joint cartilage and improved structural integrity of the joint after oral treatment of OCD rats with Alpinia:Pepper (AP) and Alpinia:Magnolia:Kochia (AMK) compositions. Natural dietary supplements exampled by, but not limited to, AP and AMK compositions increased anabolic activity by regulating homeostasis of chondrocytes, extracellular matrix, articular cartilage, and phenotype of joint. These supplements could, in fact, assist in faster recovery of damaged cartilage and improved joint structure integrity by enhancing the body's cartilage regeneration process. The specific regeneration, renewal, rebuilding and regrowth functions are associated with, but not limited to, filling of the defect relative to the surface of normal adjacent cartilage, integrating repair tissue with surrounding articular cartilage, regenerating extra cellular matrix, improving cellular morphology, renewing architecture within the entire defect, regenerating architecture of surface, increasing percentage of new subchondral bone, and enhancing formation of tidemarks. FIG. 4 shows Safranin O stain of the subchondral bone of OCD rats at the drill site. The black circle indicates the drill site for representative animal histopathology slides.

TABLE 18
Sellers cartilage regeneration parameters from treatment groups

| | | | | | | | | | | |
|---|---|---|---|---|---|---|---|---|---|---|
| | Dose | | | | | | | | | |
| Group | (mg/kg) | N | A | B | C | D | E | F | G | H |

Sellers cartilage regeneration parameters

| | | | | | | | | | | |
|---|---|---|---|---|---|---|---|---|---|---|
| Control | 0 | 10 | 0 ± 0 | 0 ± 0 | 0 ± 0 | 0 ± 0 | 0 ± 0 | 0.09 ± 0.29 | 0.09 ± 0.29 | 0.27 ± 0.45 |
| Vehicle | 0 | 10 | 1.60 ± 1.02 | 2.40 ± 1.02 | 3.70 ± 0.46 | 4.22 ± 0.63 | 1.80 ± 1.25 | 2.33 ± 0.94 | 2.80 ± 1.54 | 3.22 ± 1.03 |
| Piascledine | 200 | 10 | 1.40 ± 0.70 | 0.90 ± 0.57 | 3.90 ± 0.32 | 4.40 ± 0.70 | 1.40 ± 0.70 | 1.50 ± 0.71 | 2.30 ± 0.82 | 3.00 ± 0.94 |
| AMK | 200 | 10 | 1.10 ± 0.99 | 0.90 ± 0.88 | 2.48 ± 1.71 | 2.20 ± 1.75 | 1.03 ± 0.95 | 1.30 ± 1.06 | 1.73 ± 1.32 | 1.83 ± 1.38 |
| AP | 200 | 10 | 0.82 ± 0.58 | 0.64 ± 0.98 | 2.64 ± 1.23 | 2.09 ± 1.62 | 1.00 ± 0.74 | 1.45 ± 0.99 | 1.73 ± 1.21 | 2.18 ± 1.19 |

P-values vs Vehicle treated OCD model

| | | | | | | | | | | |
|---|---|---|---|---|---|---|---|---|---|---|
| Control | 0 | 10 | — | — | — | — | — | 0.000 | 0.000 | 0.000 |
| Piascledine | 200 | 10 | 0.628 | 0.001 | 0.288 | 0.579 | 0.449 | 0.049 | 0.396 | 0.640 |
| AMK | 200 | 10 | 0.295 | 0.003 | 0.044 | 0.005 | 0.151 | 0.044 | 0.123 | 0.027 |
| AP | 200 | 10 | 0.051 | 0.001 | 0.024 | 0.002 | 0.102 | 0.071 | 0.106 | 0.066 |

A-
B-Filling of the defect relative to surface of normal adjacent cartilage,
C-Integration of repair tissue with surrounding articular cartilage,
D-Matrix staining with Safranin 0-fast green,
D-Cellular morphology,
E-Architecture within entire defect,
F-Architecture of surface,
G-Percentage of new subchondral bone,
H-Formation of tidemark.

TABLE 19
Cartilage Repair Score According to Sellers method of histopathology analysis

| A | | | | | | | |
|---|---|---|---|---|---|---|---|
| Filling of | B | | | | | | |
| the defect | Integration | | | E | | | |
| relative | of repair | | D | Architecture | | G | |
| to surface | tissue with | | Cellular | within entire | F | Percentage | H |
| of normal | surrounding | C | morphology | defect (not | Architecture | of new | Formation |
| adjacent | articular | Matrix | (a-b-c-d/ | including | of | subchondral | of |
| cartilage | cartilage | staining | 0 thru 5) | margins) | surface | bone | tidemark |
| 111%-125% = 1 | Normal | Normal = 0 | (a) Normal = 0 | Normal = 0 | Normal = 0 | 90%-100% = 0 | Complete = 0 |
| | continuity | | | | | | |
| | and | | | | | | |
| | integration = 0 | | | | | | |
| 91%-110% = 0 | Decreased | Slightly | (b) Mostly | 1-3 | Slight | 75%-89% = 1 | 75%-99% = 1 |
| | cellularity = 1 | reduced = 1 | round cells | small | fibrillation | | 50%-74% = 2 |
| | | | with the | voids = 1 | or | | 25%-49% = 3 |
| | | | morphology | | irregu- | | <25% = 4 |
| | | | of | | larity = 1 | | |
| | | | chondrocytes | | | | |
| 76%-90% = 1 | Gap | Moderately | >75% | 1-3 | 50%-74% = 2 | 50%-74% = 2 | |
| | or lack | reduced = 2 | of tissue | large | | | |
| | of continuity | | with | voids = 2 | | | |
| | on one | | columns | | | | |
| | side = 2 | | in radial | | | | |
| | | | zone = 0 | | | | |
| 51%-75% = 2 | Gap | Substantially | 25%-75% of | >3 large | Severe | 25%-49% = 3 | |
| | or lack | reduced = 3 | tissue with | voids = 3 | fibrillation | | |
| | of continuity | | columns in | | or | | |
| | on two | | radial zone = 1 | | disruption = 3 | | |
| | sides = 3 | | | | | | |
| 26%-50% = 3 | | None = 4 | <25% | Clefts or | | <25% = 4 | |
| | | | of tissue | fibrill- | | | |
| | | | with | ations = 4 | | | |
| | | | columns | | | | |
| | | | in radial | | | | |
| | | | zone (dis- | | | | |
| | | | organized) = 2 | | | | |
| <25% = 4 | | | (c) 50% round | | | | |
| | | | cells with the | | | | |
| | | | morphology of | | | | |
| | | | chondrocytes = 1 | | | | |
| | | | >75% of | | | | |
| | | | tissue | | | | |
| | | | with | | | | |
| | | | columns | | | | |
| | | | in radial | | | | |
| | | | zone = 2 | | | | |
| | | | 25%-75% | | | | |
| | | | of | | | | |
| | | | tissue | | | | |
| | | | with | | | | |
| | | | columns | | | | |
| | | | in radial | | | | |
| | | | zone = 3 | | | | |
| | | | <25% of | | | | |
| | | | with tissue | | | | |
| | | | columns | | | | |
| | | | in radial | | | | |
| | | | zone = 4 | | | | |
| | | | (disorganized) | | | | |
| | | | d) Mostly | | | | |
| | | | spindle-shape | | | | |
| | | | (fibroblast- | | | | |
| | | | like) cells = 5 | | | | |

Orth P, Zurakowski D, Wincheringer D, Madry H. Reliability, reproducibility, and validation of five major histological scoring systems for experimental articular cartilage repair in the rabbit model. Tissue Eng Part C Methods. 2012 May; 18(5):329-39.

### Example 30. Accelerated Healing as Measured by Sellers Cartilage Regeneration Histopathology Analysis Method for Tissues from the OCD Model

We also analyzed data from the histopathology by summing up the overall parameter changes observed in the healing process from A to H. It was found that when rats were treated with Alpinia:Pepper (AP) and Alpinia:Magnolia:Kochia (AMK) compositions (Example 14 and 15) daily at 200 mg/kg for 6 weeks, there was 40.5% and 40.4% increase in healing than OCD rats in the vehicle treated group, respectively (Table 20). As seen in this data summary, it is clearly evident that the OCD model was induced and significant improvement in healing (and hence cartilage repair, regeneration, renewal, rebuilding) was observed as a result of the AMK and AP oral treatment. For comparison, the Piascledine group showed only a 10.9% faster healing process compared to the vehicle-treated OCD rats.

TABLE 20
Accelerated healing of cartilage according
to Sellers histopathology analysis

| | | | Sellers | P-value | | P-value |
|---|---|---|---|---|---|---|
| | | | cartilage | vs | | vs |
| | | | regeneration | Vehicle | | Vehicle |
| | | | overall | treated | % | treated |
| | Dose | | value | OCD | accelerated | OCD |
| Group | (mg/kg) | N | (A to H) | model | healing | model |
| Control | 0 | 11 | 0.46 ± 0.89 | 0.0000 | 97.8 | 0.0000 |
| Vehicle | 0 | 11 | 21.10 ± 4.32 | — | — | — |
| Piascledine | 200 | 11 | 18.80 ± 4.24 | 0.258 | 10.9 | 0.258 |
| AMK | 200 | 11 | 12.57 ± 8.81 | 0.014 | 40.4 | 0.014 |
| AP | 200 | 11 | 12.55 ± 7.24 | 0.006 | 40.5 | 0.006 |

### Example 31. TGF-β1 as an Anabolic Regulator of Cartilage Synthesis in OCD Rats

For biomarker analysis of OCD study illustrated in examples—27-29, cardiac blood was collected for each animal at necropsy. Blood was spun at 3000 rpm for 15 min. About 700-800 μl of serum was isolated from each rat. Samples were kept at −80° C. until use. The presence of TGF-β1 in rat serum was measured using the Rat TGF-β1 Quantikine ELISA kit from RandD Systems (product #: MB100B) as follows: Latent TGF-β1 in serum was activated with 1 N HCl, then neutralized with 1.2 N NaOH/0.5 M HEPES. Activated serum was diluted 60-fold and added to a microplate coated with TGF-β1 antibody (final dilution factor of serum is 90). After 2 hours at room temperature, TGF-β 1 in serum was bound to the plate and the plate was thoroughly washed. Enzyme-conjugated TGF-β1 antibody was added to the plate and allowed to bind for 2 hours at room temperature. The washing was repeated, and enzyme substrate was added to the plate. After developing for 30 minutes at room temperature, a stop solution was added, and the absorbance was read at 450 nm. The concentration of TGF-β1 was calculated based on the absorbance readings of a TGF-β1 standard curve.

As seen in Table 21, below, there was a statistically significant increase in the serum level of TGF-β1 in the AP-treated group compared either to the vehicle-treated OCD model or normal control rats. These increases were found to be 19.5% and 17.1% for AP composition and 9.5% and 7.3% for AMK composition relative to the normal control and the vehicle-treated OCD group, respectively. The TGF-β1 increase in the AMK-treated group was not significant compared to the vehicle-treated group.

TABLE 21
TGF-β1 as an anabolic regulator of Cartilage
synthesis in OCD rats treated with Alpinia:Pepper (AP)
and Alpinia:Magnolia:Kochia (AMK) compositions

| | | | TGF-β1 (pg/mL) | | | | |
|---|---|---|---|---|---|---|---|
| | | | | | % | | p- |
| | | | | | change | | values |
| | Dose | | | | Vs | Vs | vs |
| Group | (mg/kg) | N | Average | SE | Vehicle | control | Vehicle |
| Control | 0 | 11 | 74639.8 | 2928.9 | −2 | — | 0.74 |
| Vehicle | 0 | 11 | 76187.5 | 3587.9 | — | 2.1 | — |
| Piascledine | 200 | 11 | 83777.3 | 5054.8 | 10 | 12.2 | 0.24 |
| AMK | 200 | 11 | 81722.2 | 6381.3 | 7.3 | 9.5 | 0.46 |
| AP | 200 | 11 | 89230.2 | 3606.4 | 17.1 | 19.5 | 0.02 |

Among the anabolic biomarkers, one of the most relevant indicators of cartilage synthesis, TGF-β 1, was found elevated in the AP and AMK composition-treated rats to the point where the increase was statistically significant compared to vehicle treated OCD rats for the AP group. Significant published data support the fact that this anabolic factor is known to be involved in the maintenance of cartilage homeostasis and to stimulate cartilage repair processes by chondrocytes. While the level of TGF-β1 is high in healthy cartilage, its expression is low in patients with OA. In experimental animals with arthritis, the injection of TGF-β1 into the knee increased the level of proteoglycan while protecting against cartilage loss in others, suggesting its importance in rebuilding and homeostasis of the extracellular matrix components of the articular cartilage (van Beuningen et al., 1994; Verdier et al., 2003; Glansbeek et al., 1998). Therefore, these noticeable changes observed in our study demonstrated tipping the balance to the anabolic direction by either Alpinia:Piper/Pepper (AP) and Alpinia:Magnolia:Kochia (AMK) composition that could be associated with regulating homeostasis of chondrocytes, extracellular matrix, articular cartilage, and phenotype of a damaged joint with treatment effect much faster than the spontaneous recovery observed for the vehicle-treated OCD group.

### Example 32. Symptom Relief Function of Individual Alpinia, Pepper, Magnolia and Kochia Extracts in Carrageenan-Induced Paw Edema Model

Carrageenan-induced paw edema in rats was used to evaluate the anti-inflammatory and anti-pain activities of individual Alpinia, Pepper, Magnolia and Kochia extracts. Sprague Dawley (SD) rats (N=5 per group) were given Alpinia, Pepper, Magnolia and Kochia extracts at 300 mg/kg orally one hour after intra-plantar injection of 100 μl carrageenan. Pain sensitivity and paw edema were monitored at T0 (before carrageenan) and 2, 4 and 6 hours after carrageenan. Ibuprofen as a positive control was used at 150 mg/kg. As seen in Table 22 below, ranges of percent reductions, such as 26.1-37.3%, 7.5-33.2%, 1-14.7% and 17.9-32.3% in paw edema and 21.2-33.8%, 15.3-27.1%, 18-26.3% and 23.2-33.2% in pain sensitivity were observed for rats treated with the extracts from Alpinia, Magnolia, Kochia and Pepper, respectively. Except for in paw edema measurements 5 hr after Kochia, all treatment groups showed statistically significant reductions of pain and inflammation.

TABLE 22
Decrease of paw edema and reduction of pain sensitivity of carrageenan rats treated
with Alpinia:Piper/Pepper (AP) and Alpinia:Magnolia:Kochia (AMK) compositions

| | | | | | | | % change | | |
|---|---|---|---|---|---|---|---|---|---|
| | Average | | | SD | | | of vehicle | | |
| Group | 1 hr | 3 hr | 5 hr | 1 hr | 3 hr | 5 hr | 1 hr | 3 hr | 5 hr |

Paw edema

| | | | | | | | | | |
|---|---|---|---|---|---|---|---|---|---|
| Vehicle | 2.51 | 3.08 | 2.80 | 0.19 | 0.16 | 0.08 | — | — | — |
| Ibuprofen | 1.43 | 1.59 | 1.77 | 0.40 | 0.30 | 0.37 | 43.03 | 48.38 | 36.79 |
| Alpinia Galanga | 1.69 | 1.93 | 2.07 | 0.11 | 0.32 | 0.20 | 32.67 | 37.34 | 26.07 |
| Magnolia officinalis | 1.99 | 2.06 | 2.59 | 0.33 | 0.45 | 0.60 | 20.88 | 33.18 | 7.50 |
| Kochia Scoparia | 2.14 | 2.64 | 2.78 | 0.26 | 0.23 | 0.33 | 14.74 | 14.16 | 0.71 |
| Pepper Nigrum | 1.70 | 2.13 | 2.30 | 0.23 | 0.51 | 0.56 | 32.27 | 30.84 | 17.86 |

Pain sensitivity

| | | | | | | | | | |
|---|---|---|---|---|---|---|---|---|---|
| Vehicle | 46.75 | 65.87 | 68.45 | 2.57 | 2.25 | 2.56 | — | — | — |
| Ibuprofen | 25.02 | 35.67 | 45.49 | 3.49 | 3.42 | 4.28 | 46.49 | 45.85 | 33.55 |
| Alpinia Galanga | 30.95 | 44.02 | 53.93 | 2.11 | 2.58 | 3.19 | 33.79 | 33.17 | 21.21 |
| Magnolia Officinalis | 36.73 | 48.05 | 58.01 | 1.34 | 2.74 | 3.53 | 21.43 | 27.05 | 15.25 |
| Kochia Scoparia | 34.75 | 48.53 | 56.16 | 3.77 | 4.45 | 2.91 | 25.67 | 26.33 | 17.96 |
| Pepper Nigrum | 31.22 | 44.37 | 52.55 | 4.10 | 6.53 | 7.45 | 33.22 | 32.64 | 23.24 |

### Example 33. Lead Composition-Finding Study on Carrageenan-Induced Paw Edema Model

Carrageenan-induced paw edema model was used to evaluate the anti-inflammatory and anti-pain activities of natural compositions that were combinations of individual plant extracts from Alpinia and Magnolia at blending ratios of 1:1 (150:150 mg/kg), 1:2 (100:200 mg/kg), 2:1 (200:100 mg/kg), 1:4 (60:240 mg/kg) and 4:1 (240:60 mg/kg). Rats were administered with the compositions at the same dosage of 300 mg/kg orally. As seen in Table 23 below, significant inhibition in pain and inflammation was observed for all the ratios tested for these medicinal plant combinations. Slightly higher inhibition relative to the other ratios was found when rats were treated with a ratio of 1:2 Alpinia:Magnolia. Alpinia and Magnolia combination at 1:2 ratio showed 36.7%, 33.3% and 29.3% in pain reduction and 37.2%, 34.5% and 29.3% in reduction of inflammation at 1 h, 3 hrs and 5 hrs after treatment, respectively, when compared to the vehicle-treated disease model. In this study, we observed that combining these two medicinal plant extracts at this specific ratio produced higher inhibition of pain and inflammation (in the first hours and at 5 hr after treatment) when compared to each individual plant administered at the same dosage (compared to data from example 32 above). As a result, we selected the composition as example, but not limited to, 1:2 ratio of Alpinia with Magnolia for more subsequent studies.

TABLE 23
Anti-pain and anti-inflammatory activity of various
ratios of Alpinia/Magnolia extracts

| | | | | | | Anti- | | |
|---|---|---|---|---|---|---|---|---|
| | | | Anti-pain | | | inflammation | | |
| | Dose | | Percent change* | | | Percent change* | | |
| Extracts | (mg/kg) | Ratio | 1 hr | 3 hr | 5 hr | 1 hr | 3 hr | 5 hr |
| Ibuprofen | 150 | 1 | 50.51 | 46.37 | 37.42 | 49.50 | 48.62 | 37.66 |
| Alpinia to magnolia | 300 | 1 to 1 | 29.26 | 34.41 | 21.04 | 28.15 | 28.40 | 25.04 |
| Alpinia to magnolia | 300 | 1 to 2 | 36.71 | 33.30 | 29.27 | 37.20 | 34.53 | 29.31 |
| Alpinia to magnolia | 300 | 2 to 1 | 33.52 | 34.38 | 25.52 | 32.53 | 31.60 | 25.99 |
| Alpinia to magnolia | 300 | 4 to 1 | 27.76 | 30.30 | 24.28 | 23.55 | 27.02 | 19.66 |
| Alpinia to magnolia | 300 | 1 to 4 | 15.22 | 16.90 | 11.68 | 12.52 | 15.86 | 15.04 |

* P ≤ 0.05 compared to vehicle

### Example 34. Anti-Pain and Anti-Inflammatory Activities of Combinations of Alpinia galanga and Kochia scoparia in the Carrageenan-Induced Paw Edema Model

Carrageenan-induced paw edema model was used to evaluate the anti-inflammatory and anti-pain activities of Alpinia and Kochia extracts, which were combined at 1:1 (150:150 mg/kg), 1:2 (100:200 mg/kg), 2:1 (200:100 mg/kg), 1:4 (60:240 mg/kg) and 4:1 (240:60 mg/kg) ratios. Rats were administered with the compositions at 300 mg/kg orally in total. As seen in Table 24 below, significant inhibition in pain and inflammation was observed for all the ratios tested for these medicinal plants. A slightly higher inhibition relative to the other ratios was found when rats were tested with a 1:1 Alpinia to Kochia ratio followed by 4:1 Alpinia to Kochia ratio. Alpinia and Kochia extract's combination at 1:1 ratio showed 25.0%, 27.6% and 25.7% in pain reduction and 26.4%, 30.9% and 30.6% in reduction of inflammation at 1 h, 3 hrs and 5 hrs after treatment, respectively, when compared to vehicle-treated disease model. Similarly, the Alpinia and Kochia extract combination at 4:1 ratio showed 20.4%, 26.9% and 26.3% in pain reduction and 24.1%, 30.7% and 29.1% in reduction of inflammation at 1 h, 3 hrs and 5 hrs after treatment, respectively, when compared to the vehicle-treated disease model.

TABLE 24
Anti-pain and anti-inflammatory activity of various ratios of Alpinia and Kochia extracts

| | | | | | | Anti- | | |
|---|---|---|---|---|---|---|---|---|
| | | | Anti-pain | | | inflammation | | |
| | Dose | | Percent change* | | | Percent change* | | |
| Extracts | (mg/kg) | Ratio | 1 hr | 3 hr | 5 hr | 1 hr | 3 hr | 5 hr |
| Ibuprofen | 100 | 1 | 50.51 | 46.37 | 37.42 | 50.07 | 47.40 | 37.28 |
| Alpinia to Kochia | 300 | 1 to 1 | 24.96 | 27.56 | 25.72 | 26.36 | 30.88 | 30.55 |
| Alpinia to Kochia | 300 | 1 to 2 | 21.70 | 23.25 | 24.63 | 26.50 | 29.86 | 29.66 |
| Alpinia to Kochia | 300 | 2 to 1 | 12.19 | 21.26 | 14.40 | 11.75 | 20.31 | 10.90 |
| Alpinia to Kochia | 300 | 1 to 4 | 13.08 | 18.21 | 21.49 | 13.71 | 22.00 | 23.41 |
| Alpinia to Kochia | 300 | 4 to 1 | 20.36 | 26.92 | 26.27 | 24.13 | 30.66 | 29.06 |

*P ≤ 0.05 compared to vehicle

### Example 35. Symptom Relief Effects of Composition AMK (Alpinia, Magnolia and Kochia)

We demonstrate in this example, but not limited to it, that adding a third component to an Alpinia:Magnolia composition further increased the efficacy of the composition. The Kochia scoparia extract with both anabolic and catabolic modulation activities on chondrocytes as demonstrated in examples 22 and 24 was selected as a third component and evaluated in the carrageenan induced rat paw edema model as shown in the next example. Carrageenan-induced paw edema was used here again for the evaluation of AM (1:2) blended with Kochia at 4:1, 2:1 and 1:1 ratios with a final dosage of 300 mg/kg. While the addition of Kochia seemed to boost the efficacy of AM in all the ratios, there was a statistically significant increase in anti-pain and anti-inflammation activity when Kochia was added at a 2:1 ratio to AM (i.e. 2A:4M:3K). There was 40.8, 45.2 and 33.1% reduction in pain and a 42.1, 37.8 and 36.0% reductions in inflammation at 1 h, 3 hrs and 5 hrs after treatment, respectively, when compared to the vehicle-treated disease model. These inhibitions were higher than for individual extracts and the Alpinia and Magnolia combination. At this dosage, the final ratio for the most effective composition was determined as 2A:4M:3K.

TABLE 25
Anti-pain and anti-inflammatory activity
of various ratios of AM and Kochia

| | | | | | | Anti- | | |
|---|---|---|---|---|---|---|---|---|
| | | | Anti-pain | | | inflammation | | |
| | Dose | | Percent change* | | | Percent change* | | |
| Extracts | (mg/kg) | Ratio | 1 hr | 3 hr | 5 hr | 1 hr | 3 hr | 5 hr |
| Ibuprofen | 150 | 1 | 44.4 | 47.9 | 30.8 | 47.3 | 49.7 | 37.6 |
| AM to K | 300 | 4 to 1 | 34.6 | 42.5 | 27.5 | 36.7 | 40.3 | 34.2 |
| AM to K | 300 | 2 to 1 | 40.8 | 45.2 | 33.1 | 42.1 | 37.8 | 36.0 |
| AM to K | 300 | 1 to 1 | 21.9 | 36.9 | 22.9 | 34.8 | 33.4 | 24.5 |

*P ≤ 0.05 compared to vehicle

### Example 36. Synergistic Activity of Alpinia:Magnolia:Kochia (AMK) Composition in Reducing Pain and Inflammation in Carrageenan-Induced Rat Paw Edema Model

The merit of combining Alpinia, Magnolia and Kochia for AMK (2:4:3 ratio, respectively) was further demonstrated in this example on the carrageenan rat paw edema model. Rats were gavaged with each constituent as they appeared in the 300 mg/kg of the AMK individually in order to determine whether the plant extracts acted synergistically. For the 2:4:3 ratio of AMK, rats were administered with 67 mg/kg of Alpinia extract, 133 mg/kg of Magnolia extract and 100 mg/kg of Kochia extract. The percent inhibition of pain and inflammation of the combined compositions at 300 mg/kg were compared with those dosages of individual extracts to find out potential additive, antagonistic or synergistic effects in combination using the Colby's equation.

TABLE 26
Synergistic activity of combination of Alpinia, Magnolia
and Kochia extracts in reducing pain and inflammation
in carrageenan induced rat paw edema model

| | | AMK (2:4:3) | | | | | |
|---|---|---|---|---|---|---|---|
| | | Anti- | | | | | |
| Dose | Ratio/ | inflammation | | | Anti-pain | | |
| (mg/kg) | equation | 1 hr | 3 hr | 5 hr | 1 hr | 3 hr | 5 hr |
| 67 | 2A (x) | 17.8 | 13.8 | 10.5 | 15.5 | 17.4 | 9.0 |
| 133 | 4M (y) | 22.6 | 17.2 | 12.2 | 20.6 | 23.5 | 15.0 |
| 100 | 3K (z) | 14.3 | 14.7 | 7.6 | 18.1 | 11.3 | 7.6 |
| | (x + y + Z) = A | 54.8 | 45.7 | 30.3 | 54.2 | 52.3 | 31.7 |
| | (xyz)/10000 = B | 0.6 | 0.3 | 0.1 | 0.6 | 0.5 | 0.1 |
| | ((xy) + (xz) + | 9.8 | 6.9 | 3.0 | 9.7 | 8.7 | 3.2 |
| | (yz))/100 = C | | | | | | |
| 300 | Expected | 45.5 | 39.1 | 27.4 | 45.0 | 44.0 | 28.6 |
| | Observed | 47.5 | 45.2 | 36.4 | 48.2 | 45.8 | 36.2 |

Colby's synergy equation for Expected = A − C + B

For the blending of these plant extracts to have unexpected synergy, the observed inhibitions needed to be greater than the calculated expected value. As seen in Table 26, the observed efficacies were, in fact, greater than the expected values at each time point monitored, suggesting synergy among these medicinal plant extracts in reducing pain and inflammation. Though previously reported studies indicated the potential anti-inflammatory activity of these herbs, none of them were put together in the standardized blend presented in this patent with the depicted potency.

### Example 37. AP Composition-Finding Study Using the Carrageenan-Induced Paw Edema Model

Carrageenan-induced paw edema model was used to evaluate the anti-inflammatory and anti-pain activities of Alpinia and Piper/Pepper extracts which were combined at 1:1 (150:150 mg/kg), 1:2 (100:200 mg/kg), 2:1 (200:100 mg/kg), 1:4 (60:240 mg/kg) and 4:1 (240:60 mg/kg) ratios. Rats were administered with the compositions at 300 mg/kg orally in total. As seen in Table 27, below, significant inhibition in pain and inflammation was observed for all the ratios tested for these medicinal plants. Slightly higher inhibition, relative to the other ratios, was found when rats were tested with a 1:2 Alpinia to Piper/Pepper ratio. Alpinia and Piper/Pepper extracts combined at a 1:2 ratio showed 42.4%, 44.3% and 34.7% pain reduction and 39.2%, 43.4% and 33.7% reduction of inflammation at 1 h, 3 hrs and 5 hrs after treatment, respectively, when compared to vehicle-treated disease model. This composition was selected for dose-response and synergy studies.

TABLE 27
Anti-pain and anti-inflammatory activities of
various ratios of Alpinia and Pepper

| | | | | | | Anti- | | |
|---|---|---|---|---|---|---|---|---|
| | | | Anti-pain | | | inflammation | | |
| | Dose | | Percent change* | | | Percent change* | | |
| Extracts | (mg/kg) | Ratio | 1 hr | 3 hr | 5 hr | 1 hr | 3 hr | 5 hr |
| Ibuprofen | 100 | 1 | 48.44 | 47.46 | 37.77 | 45.00 | 50.31 | 41.84 |
| Alpinia to | 300 | 1 to 1 | 31.44 | 38.21 | 24.19 | 33.31 | 33.75 | 22.86 |
| Pepper | | | | | | | | |
| Alpinia to | 300 | 1 to 2 | 42.36 | 44.33 | 34.70 | 39.23 | 43.44 | 33.67 |
| Pepper | | | | | | | | |
| Alpinia to | 300 | 2 to 1 | 33.25 | 35.42 | 21.67 | 27.69 | 18.31 | 11.16 |
| Pepper | | | | | | | | |
| Alpinia to | 300 | 1 to 4 | 33.25 | 33.93 | 31.27 | 31.23 | 34.63 | 25.17 |
| Pepper | | | | | | | | |
| Alpinia to | 300 | 4 to 1 | 35.40 | 37.25 | 23.73 | 25.08 | 29.25 | 21.77 |
| Pepper | | | | | | | | |

*P ≤ 0.05 compared to vehicle

### Example 38. Dose-Response Study of Selected AP Compositions

Composition AP was selected as the lead composition from previous in vivo experiments due to its inhibition of inflammation and pain at the 1:2 ratio, when administered orally to rats at 300 mg/kg. Here, we evaluated the dose-response effect of this combination in carrageenan—induced rat paw edema model administered at 100, 200 and 300 mg/kg. As seen in Table 28, below, dose-correlated inhibition of inflammation and pain was observed for the composition. The highest anti-inflammatory activities were observed at the 300 mg/kg for the composition followed by the 200 mg/kg and 100 mg/kg. Inhibition of 42.8%, 43.5% and 32.0% of inflammation and 44.8%, 44.4% and 34.7% of pain was observed at 1 hr, 3 hr and 5 hrs after treatment, respectively

TABLE 28
Dose response in anti-pain and anti-
inflammation activity of composition AP

| | | | | | | Anti- | | |
|---|---|---|---|---|---|---|---|---|
| | | | Anti-pain | | | inflammation | | |
| | Dose | | Percent change* | | | Percent change* | | |
| Extracts | (mg/kg) | Ratio | 1 hr | 3 hr | 5 hr | 1 hr | 3 hr | 5 hr |
| Ibuprofen | 100 | 1 | 50.5 | 46.1 | 38.5 | 51.0 | 48.0 | 39.0 |
| Alpinia to | 100 | 1 to 2 | 10.7 | 18.0 | 13.1 | 26.2 | 14.2 | 11.0 |
| Pepper | | | | | | | | |
| Alpinia to | 200 | 1 to 2 | 29.3 | 33.7 | 27.8 | 37.9 | 27.1 | 25.1 |
| Pepper | | | | | | | | |
| Alpinia to | 300 | 1 to 2 | 44.8 | 44.4 | 34.7 | 42.8 | 43.5 | 32.0 |
| Pepper | | | | | | | | |

*P ≤ 0.05 compared to vehicle

### Example 39. Synergy Determination of Lead Extracts in Composition AP

The merit of combining Alpinia with Pepper for AP (1:2 ratio) was evaluated in the carrageenan rat paw edema model. Rats were gavaged with each constituent as they appeared in 300 mg/kg of the AP. For the 1:2 ratio of AP, 100 mg/kg of Alpinia and 200 mg/kg of Pepper extract were administered to the rats. The percent inhibition of pain and inflammation of the compositions at 300 mg/kg was compared with those dosages of individual extracts to find out potential additive, antagonistic or synergistic effects in combination using Colby's equation (Colby 1967). For the blending of these plant extracts to have unexpected synergy, the observed inhibition needed to be greater than the calculated value.

TABLE 29
Unexpected synergistic activities of Alpinia
and Pepper in 1A2P combination

| Dose | | 1A2P | | | | | |
|---|---|---|---|---|---|---|---|
| (mg/ | Ratio/ | Anti-inflammation | | | Anti-pain | | |
| kg) | equation | 1 hr | 3 hr | 5 hr | 1 hr | 3 hr | 5 hr |
| 100 | 1A (x) | 16.4 | 11.9 | 7.1 | 15.34 | 21.15 | 11.63 |
| 200 | 2P (y) | 22.3 | 23.3 | 12.3 | 22.46 | 27.29 | 18.24 |
| | (x ± y) | 38.6 | 35.2 | 19.4 | 37.8 | 48.4 | 29.9 |
| | (xy)/100 | 3.6 | 2.8 | 0.9 | 3.4 | 5.8 | 2.1 |
| 300 | Expected | 34.98 | 32.43 | 18.52 | 34.35 | 42.67 | 27.75 |
| | Observed | 42.81 | 43.52 | 31.96 | 44.82 | 44.40 | 34.73 |

Colby's synergy equation for Expected:(X + Y) − XY/100

As seen in Table 29, the observed efficacies were in fact greater than the expected values at each time point monitored, suggesting the unexpected synergistic activities of these plant extracts in reducing pain and inflammation.

### Example 40. Stimulation of Cartilage Synthesis and Inhibition of Cartilage Degradation by Composition Alpinia:Magnolia:Kochia (AMK) in the Collagen-Induced Arthritis (CIA) Rat Model

Several biomarkers of bone, cartilage, and the synovium have been described, and their changes have been investigated in patients with OA for efficacy of intervention, disease prognosis, diagnosis and progression (Garnero et al., 2000). Loss of cartilage is believed to result from an imbalance of cartilage homeostasis to the catabolic direction by a combination of decreased repair processes and increased degradation activities in OA patients. Due to the limited capacity for cartilage repair and as type II collagen is the most abundant protein of the cartilage matrix, the assessment of type II collagen synthesis and degradation seemed to be a feasible approach to assess efficacy of OA interventions. For example, cartilage tissue from patients with OA and healthy controls have shown both altered synthesis and increased degradation of type II collagen (Nelson et al., 1998; Billinghurst et al., 1997).

Therefore, the use of two biomarkers each addressing the synthesis or degradation of articular cartilage (in particular type II collagen) could be used as tools to better predict either OA progression or efficacy of OA treatment. This method coupled both the anabolic and catabolic processes of articular cartilage homeostasis. During cartilage development, type II collagen is synthesized as procollagen with N- and C-propeptide terminals, and type II procollagen is produced in two forms (Type A and type B) as the result of alternative RNA splicing. The release of either of the propeptides from the synovial fluid to the blood circulation at the time of secretion and before incorporation of type II collagen into ECM can be used to determine the rate of cartilage synthesis or regeneration or rebuilding. On the other hand, urinary C-telopeptides of type II collagen (uCTX-II), is a prominent marker for cartilage degradation. Urine C-terminal telopeptide of type II collagen (uCTX-II) has been by far the most studied and frequently referred to and validated biomarker of cartilage degradation that could be used for the purpose of diagnosis, determining the severity of disease or extent of disease progression, prognosis, and monitoring efficacy of treatment (Oesterggaard et al., 2006). In clinical studies, high levels of uCTX-II are a good predictor of increased risk of joint destruction (Garnero et al., 2001).

We used two primary biomarkers, uCTX-II and PIIANP, to determine the cartilage homeostasis of degradation (and hence catabolic activity) and cartilage rebuilding (and hence anabolic activity) effects of the novel composition Alpinia:Pepper (AP) and Alpinia:Magnolia:Kochia (AMK) administered orally in the collagen-induced rat arthritis model. Previously, Garnero et al measured these markers [Type II collagen synthesis and degradation: N-propeptide of type IIA procollagen (PIIANP) and urine CTX-II, respectively] and correlated them to findings on radiographs and arthroscopy of OA patients. Their findings showed that patients with low serum levels of PIIANP and high urine levels of CTX-II had relative risks of progression of OA of 2.9 by radiography and 9.3 by arthroscopy (Garnero et al, 2002). They explained their observation that these patients have an uncoupling effect between collagen synthesis and degradation, which is leaning more towards the progression of OA.

TABLE 30
Type II collagen synthesis and degradation predicts efficacy of OA intervention.

| | | | Z score | | | | | |
|---|---|---|---|---|---|---|---|---|
| | | | | | | | Imbalance | |
| | | | Regeneration | | Degradation | | (catabolic − anabolic) | |
| | | | | P- | | P- | | P- |
| | Dose | | | values | | values | | values |
| Group | (mg/kg) | N | Mean ± SE | vs CIA | Mean ± SE | vs CIA | Mean ± SE | vs CIA |
| CIA | 0 | 10 | −1.39 ± 0.23 | — | 1.59 ± 0.31 | — | 2.98 ± 0.36 | — |
| Methotrexate | 0.5 | 10 | −0.90 ± 0.16 | 0.136 | 0.39 ± 0.34 | 0.06 | 1.30 ± 0.33 | 0.005 |
| AMK | 200 | 10 | −0.59 ± 0.20 | 0.009 | −0.07 ± 0.45 | 0.003 | 0.52 ± 0.38 | 0.0004 |

Uncoupling (imbalance) = (CTX-II)-(PIIANP)
Z score = number of SDs from the mean of normal control rats.

Adapting their method, we calculated the Z-factor for cartilage synthesis and degradation using data from PIIANP and uCTX-II, respectively. For an intervention to drive the OA catabolic progression towards anabolic or regenerative activities, the Z-score values have to come close to zero. As seen in Table 30, below, both the regeneration Z-score (AMK: −0.59±0.20 vs CIA: −1.39±0.23 showing less compromised rebuild function from AMK) and degradation Z-score values (AMK: −0.07±0.45 vs CIA: 1.59±0.31 showing less degradation from AMK) (Table 30) for AMK were significantly different from the vehicle-treated disease group (CIA group). These improvements in driving the OA progression to normalcy by inducing cartilage synthesis and protection of its degradation were found to be 82.6% and 56.4% for the AMK and Methotrexate treated animals, respectively, relative to the vehicle treated CIA rats.

### Example 41. Collagen-Induced Arthritis Model Induction and AMK Treatment

Male Sprague Dawley rats (7-8 weeks old, n=40) were purchased from Charles River Laboratories Inc. (Wilmington, Mass., USA) and acclimated upon arrival for two weeks before being assigned randomly to their respective treatment groups: G1=Normal control (−) (n=10/group), G2=collagen-induced arthritis (CIA)+Vehicle (0.5% Carboxy Methylcellulose) (n=10/group), G3=CIA+Methotrexate (+) (75 μg/kg) (n=10/group), and G4=CIA+AMK (+) (200 mg/kg) (n=10/group). Treatment was initiated two weeks before model induction and lasted for an additional three weeks thereafter. Collagen type-II (Lot #845) from bovine nasal septum and Incomplete Freund's adjuvant (IFA) (Lot #SLBR0642v) were purchased from Elastin Products Company (Owensville, Mich., USA) and Sigma (St. Louise, Mo., USA), respectively. All materials were kept at suitable temperatures as recommended by the manufacturer. At the time of preparation, 60 mg of collagen was weighed and added to pre-chilled 15 mL 0.1 M acetic acid in a 60 mL-sized flask with a magnetic stirrer to yield 4 mg/mL concentration (Brand, et al., 2003; Rosloniec et al., 2001).

The mixture was dissolved by gently stirring overnight at 4° C. The next morning, the dissolved collagen was emulsified with equal volume of IFA (15 mL) to achieve a final concentration of 2 mg/mL collagen. Rats sedated with isoflurane were then primed intradermally with 400 μL of the emulsified collagen at the base of their tail at two sites using a 1 mL syringe fitted with a 26 G needle. The dissolved mixture was kept in an ice bucket and stirred between groups at the time of injection to preserve uniform consistency. On the seventh day, rats were inoculated with a booster dose of 2 mg/mL type II collagen emulsified with equal volume of incomplete adjuvant at 100 μL/rat/site.

Clinical findings such as arthritis severity index, paw thickness, ankle diameter (using Digital Absolute, Model #PK-0505CPX, Mitutoyo Corporation, Kawasaki, Japan), and pain sensitivity (using Randall Salitto, IITC Life Science Inc., Woodland Hills, Calif., USA) were monitored during the course of study. Urine was collected from overnight fasted rats using metabolic cages after three weeks of treatment post-model induction. At Necropsy, serum from the cardiac and synovial lavage (100 μL of saline was injected into the articular cavity and aspirated back to the syringe) for biomarkers and ankle joint for histopathology were collected from each animal.

Linear trapezoid rule was used to calculate area under the curve (AUC) for days 9-21. % Inhibition={(Mean value of treatment-mean value of CIA+)/(Mean value of control-mean value of CIA)}*100.

### Example 42. Reduction of Arthritis Severity Index of Rats by Composition AMK in the CIA Model

Rats continued to show a slow progression of disease for the duration of study. As seen in the data, rats treated with Methotrexate and AMK showed statistically significant suppression of arthritis severity from day 12 and continued this significance for the duration of study (Table 31).

At the end of the study, average severity scores of 3.75±0.32, 1.78±0.79, and 1.95±1.17, were observed for rats treated with Vehicle, Methotrexate, and AMK, respectively. It demonstrated a clear separation of the effect and potency of AMK and Methotrexate treatments. When the area under the arthritis severity curve was calculated, the percent reductions of 62.55% (p=0.04) and 51.35% (p=0.04) with statistical significance were observed from positive control Methotrexate and AMK treatment (Table 31), respectively.

### Example 43. Reduction of the Ankle Diameters of CIA Rats by Composition AMK, Indicating its Anti-Arthritic Activity

In agreement with the severity score, rats treated with Methotrexate and AMK showed a statistically significant reduction in ankle diameter starting from day 12 and maintained this significance for the duration of study (Table 32). These groups showed a statistically significant reduction in ankle width when the area under the curve was considered for days 9 to 21. Percent reductions of 65.94% and 55.84% with statistical significance in ankle diameter were observed for rats treated with Methotrexate and AMK, respectively (Table 32).

### Example 44. Reduction of the Paw Thickness of CIA Rats by Composition AMK, Indicating its Anti-Arthritic Activity

In agreement with the severity score and ankle diameter, rats treated with Methotrexate and AMK showed a statistically significant reduction in paw swelling starting from day 12; this significance was maintained for the duration of the study (Table 33). When the total area under the swelling curve (day 12-day 21) was considered, Methotrexate and AMK groups showed statistically significant reductions (71.7% and 64.3%) in paw edema compared to the vehicle treated CIA group, respectively (Table 34).

### Example 45. Arthritis Index, Ankle Diameter and Area Under the Response Curve (AUC) for Paw Thickness of CIA Rats Treated with Composition Alpinia:Magnolia:Kochia (AMK)

As seen in Table 34, above, rats treated with AP showed 64.23%, 55.8% and 51.4% inhibition in paw thickness, ankle diameter and arthritis severity index during the course of the study period when compared to vehicle-treated CIA rats, respectively. These reductions were more than 50% in each parameter and statistically significant for each parameter, indicating the potency of composition AMK in reducing arthritis-associated symptoms. In comparison, the Methotrexate-treated rats showed 71.7%, 65.9% and 62.6% reduction in paw thickness, ankle diameter and arthritis severity index, respectively.

### Example 46. Reduction of Compression-Induced Pain in CIA Rats by Composition Alpinia:Magnolia:Kochia (AMK) Indicating its Symptom Relief Activity

Response to pressure as a measure of pain sensitivity was assessed using the Randall-Salitto probe attached to an electronic monitor on priming day, boost day, and days 12, 14, 16, 19, and 21. Both the left and right hind legs were monitored on those days and their average was used for data analysis. Changes from the vehicle-treated CIA rats have been reported as pain tolerance on those days. The highest pain tolerance was observed for rats in the Methotrexate group followed by the AMK group (Table 35) in the disease models. These reductions, 6.8%, 13.5%, 28.2, 40.8%,and 43.9% for Methotrexate and 6.9%, 17.5%, 23.2%, 32.4% and 39.0% for AMK on days 12, 14, 16, 19, and 21, respectively, were statistically significant as of day 12 and remained significant for the duration of the study except on day 14 for the Methotrexate group, when reduction was not statistically significant.

TABLE 31
Changes of Arthritis severity index for rats treated with AMK in CIA model:

| | | Arthritis Index | | | | | | | | | | | | |
|---|---|---|---|---|---|---|---|---|---|---|---|---|---|---|
| | | | | Day | Day | Day | Day | Day | Day | Day | Day | day | day | day |
| Groups | Stat | Priming | Boost | 9 | 10 | 11 | 12 | 13 | 14 | 15 | 16 | 17 | 19 | 21 |
| NC | Mean | 0.00 | 0.00 | 0.00 | 0.00 | 0.00 | 0.00 | 0.00 | 0.00 | 0.00 | 0.00 | 0.00 | 0.00 | 0.00 |
| | Sd | 0.00 | 0.00 | 0.00 | 0.00 | 0.00 | 0.00 | 0.00 | 0.00 | 0.00 | 0.00 | 0.00 | 0.00 | 0.00 |
| CIA+ | Mean | 0.00 | 0.00 | 0.00 | 0.03 | 0.15 | 0.88 | 1.49 | 2.11 | 2.86 | 3.60 | 3.78 | 3.95 | 3.75 |
| | Sd | 0.00 | 0.00 | 0.00 | 0.08 | 0.25 | 0.63 | 0.42 | 0.36 | 0.30 | 0.34 | 0.17 | 0.10 | 0.32 |
| MTX | Mean | 0.00 | 0.00 | 0.00 | 0.00 | 0.00 | 0.23* | 0.46^{¥} | 0.70^{¥} | 1.10^{€} | 1.50^{€} | 1.76^{€} | 2.03^{€} | 1.78^{€} |
| (75 μg/kg) | Sd | 0.00 | 0.00 | 0.00 | 0.00 | 0.00 | 0.36 | 0.52 | 0.71 | 0.74 | 0.81 | 0.91 | 1.03 | 0.79 |
| AMK | Mean | 0.00 | 0.00 | 0.00 | 0.03 | 0.05 | 0.28* | 0.71^{†} | 0.98^{¥} | 1.48^{€} | 1.80^{€} | 2.03^{€} | 2.15^{€} | 1.95^{¥} |
| (200 mg/kg) | Sd | 0.00 | 0.00 | 0.00 | 0.08 | 0.15 | 0.44 | 0.50 | 0.62 | 0.58 | 0.71 | 0.67 | 1.02 | 1.17 |

*P ≤ 0.05; ^{†}P ≤ 0.001; ^{¥}P ≤ 0.0001; ^{€}P ≤ 0.00001
0: No sign of grossly visible arthritis (Normal)
1: Swelling and/or redness of one or two interphalangeal joints (or mild swelling and erythema of digits or ankles)
2: Involvements of three to four interphalangeal joints (or moderate swelling and erythema of digits or ankles)
3: Swelling of entire paw (or marked swelling of paws including digits)
4: Deformity and ankylosis (or sever swelling and erythema with limited motion in many joints)

TABLE 32
Changes in ankle width for CIA rats treated with Alpinia:Magnolia:Kochia (AMK),

| | | Ankle diameter | | | | | | | | | | | | |
|---|---|---|---|---|---|---|---|---|---|---|---|---|---|---|
| | | Prim- | | Day | Day | Day | Day | Day | Day | Day | Day | day | day | day |
| Groups | Stat | ing | Boost | 9 | 10 | 11 | 12 | 13 | 14 | 15 | 16 | 17 | 19 | 21 |
| NC | Mean | 6.02 | 6.06 | 6.08 | 6.09 | 6.06 | 6.03* | 6.07^{¥} | 6.11^{€} | 6.09^{€} | 6.08^{€} | 6.11^{€} | 6.02^{€} | 6.06^{€} |
| | Sd | 0.04 | 0.03 | 0.02 | 0.03 | 0.02 | 0.03 | 0.02 | 0.02 | 0.02 | 0.03 | 0.05 | 0.04 | 0.03 |
| CIA+ | Mean | 6.06 | 6.07 | 6.29 | 6.51 | 6.98 | 7.21 | 7.62 | 8.50 | 8.65 | 9.25 | 9.45 | 6.06 | 6.07 |
| | Sd | 0.10 | 0.03 | 0.20 | 0.40 | 0.48 | 0.80 | 0.76 | 0.84 | 1.00 | 1.20 | 1.17 | 0.10 | 0.03 |
| MTX | Mean | 6.03 | 6.08 | 6.12 | 6.17 | 6.33 | 6.50* | 6.67* | 6.85* | 7.06* | 7.28* | 7.32* | 6.03† | 6.08† |
| (75 μg/kg) | Sd | 0.10 | 0.06 | 0.11 | 0.22 | 0.46 | 0.71 | 1.00 | 1.30 | 1.30 | 1.32 | 1.16 | 0.10 | 0.06 |
| AMK | Mean | 6.00 | 6.01 | 6.10 | 6.19 | 6.37 | 6.54* | 6.86† | 7.18* | 7.44* | 7.69* | 7.65* | 6.00* | 6.01* |
| (200 mg/ | Sd | 0.06 | 0.04 | 0.11 | 0.21 | 0.27 | 0.35 | 0.64 | 1.14 | 1.33 | 1.75 | 1.70 | 0.06 | 0.04 |
| kg) | | | | | | | | | | | | | | |

*P ≤ 0.05; †P ≤ 0.001, ^{¥}P ≤ 0.0001; ^{€}P ≤ 0.00001

TABLE 33
Changes in paw thickness as a measure of anti-arthritic effect of
Alpinia:Magnolia:Kochia (AMK), in CIA rats

| | | Paw thickness | | | | | | | | | | | | |
|---|---|---|---|---|---|---|---|---|---|---|---|---|---|---|
| | | | | Day | Day | Day | Day | Day | Day | Day | Day | Day | Day | Day |
| Groups | Stat | Priming | Boost | 9 | 10 | 11 | 12 | 13 | 14 | 15 | 16 | 17 | 19 | 21 |
| NC | Mean | 2.97 | 2.97 | 3.01 | 3.10 | 3.12 | 3.04* | 3.05^{¥} | 3.07^{€} | 3.07^{€} | 3.08^{€} | 3.08^{€} | 2.97^{€} | 2.97^{€} |
| | Sd | 0.06 | 0.05 | 0.04 | 0.05 | 0.04 | 0.04 | 0.03 | 0.05 | 0.03 | 0.03 | 0.04 | 0.06 | 0.05 |
| CIA+ | Mean | 3.03 | 3.10 | 3.30 | 3.50 | 3.87 | 4.24 | 4.79 | 5.34 | 5.77 | 6.20 | 5.84 | 3.03 | 3.10 |
| | Sd | 0.05 | 0.05 | 0.21 | 0.43 | 0.39 | 0.56 | 0.61 | 0.97 | 0.85 | 0.80 | 0.77 | 0.05 | 0.05 |
| MTX | Mean | 3.06 | 3.07 | 3.12 | 3.17 | 3.32 | 3.47* | 3.53* | 3.60* | 3.98^{†} | 4.32^{†} | 3.96^{¥} | 3.06^{¥} | 3.07^{€} |
| (75 μg/kg) | Sd | 0.05 | 0.06 | 0.07 | 0.15 | 0.40 | 0.67 | 0.73 | 0.79 | 0.85 | 0.93 | 0.69 | 0.05 | 0.06 |
| AMK | Mean | 3.05 | 3.03 | 3.10 | 3.17 | 3.35 | 3.52* | 3.66* | 3.85* | 4.26^{†} | 4.56^{†} | 4.50^{†} | 3.05^{†} | 3.03* |
| (200 mg/ | Sd | 0.05 | 0.05 | 0.10 | 0.21 | 0.37 | 0.53 | 0.54 | 0.75 | 0.91 | 1.20 | 1.20 | 0.05 | 0.05 |
| kg) | | | | | | | | | | | | | | |

*P ≤ 0.05; ^{†}P ≤ 0.001; ^{¥}P ≤ 0.0001; ^{€}P ≤ 0.00001

TABLE 34
Area under the response curve for CIA rats treated with
Alpinia:Magnolia:Kochia (AMK)

| | AUC_{(d9-d21)} | | | | | | | | |
|---|---|---|---|---|---|---|---|---|---|
| | (Mean ± SD) | | | % Inhibition | | | P-values VS CIA + vehicle | | |
| | Paw | Ankle | Arthritis | Paw | Ankle | Arthritis | Paw | Ankle | Arthritis |
| Group | thickness | diameter | index | thickness | diameter | index | thickness | diameter | index |
| CIA+ | 26.25 ± 0.75 | 44.29 ± 0.75 | 13.00 ± 0.75 | — | — | — | — | — | — |
| MTX | 20.64 ± 0.21 | 39.11 ± 0.21 | 4.87 ± 0.21 | 71.71 | 65.94 | 62.55 | 0.01 | 0.02 | 0.04 |
| (75 μg/kg) | | | | | | | | | |
| AMK | 21.22 ± 0.31 | 39.91 ± 0.31 | 6.33 ± 0.31 | 64.26 | 55.84 | 51.35 | 0.03 | 0.05 | 0.04 |
| (200 mg/kg) | | | | | | | | | |

TABLE 35
Anti-pain activity of Alpinia:Magnolia:Kochia (AMK) in CIA rats

| | Compression pain resistance vs CIA + vehicle | | | | | | |
|---|---|---|---|---|---|---|---|
| | (Mean ± SD) | | | | | | |
| Group | Prime | Boost | Day 12 | Day 14 | Day 16 | Day 19 | Day 21 |
| Normal | 0.83 ± 3.05 | 0.79 ± 2.56 | 14.71 ± 1.17^{†} | 50.62 ± 2.22^{¥} | 84.9 ± 2.07^{€} | 139.54 ± 2.81^{€} | 142.45 ± 1.69^{€} |
| control | | | | | | | |
| MTX | −0.12 ± 1.39 | 0.27 ± 2.16 | 6.75 ± 4.56* | 13.48 ± 15.98 | 28.18 ± 12.53^{†} | 40.77 ± | 43.88 ± 5.97^{¥} |
| (75 μg/kg) | | | | | | 10.91^{€} | |
| AMK | −0.54 ± 2.34 | −0.07 ± 2.60 | 6.85 ± 6.24* | 17.52 ± 14.17* | 23.24 ± 11.45^{†} | 32.36 ± 9.80^{¥} | 39.03 ± 7.51^{¥} |
| (200 | | | | | | | |
| mg/kg) | | | | | | | |

*P ≤ 0.05; ^{†}P ≤ 0.001; ^{¥}P ≤ 0.0001; ^{€}P ≤ 0.00001

### Example 47. Reduction of Proinflammatory Cytokines and Matrix Degrading Enzymes by Composition Alpinia:Magnolia:Kochia (AMK) in CIA Rats

The presence of catabolic cytokines IL-1β, TNF-α, or IL-6 was measured using Rat IL-1, TNF-α, and IL-6 Quantikine ELISA kit from R and D Systems (product #: RLB00 for IL-1β, RTA00 for TNF-α, and R6000B for IL-6) as follows: diluted serum was added to a microplate coated with polyclonal IL-1, TNF-α, or IL-6 antibody and allowed to bind for 2 h at room temperature. The microplate was washed thoroughly to remove unbound serum and then a polyclonal enzyme-conjugated IL-1β, TNF-α, or IL-6 antibody was added and allowed to bind for 2 h at room temperature. Washing was repeated, enzyme substrate was added, and the plate was developed for 30 min at room temperature. After the addition of stop solution, the absorbance was read at 450 nm, multiplied by dilution factor, and the concentration of IL-1β/TNF-α/IL-6 calculated based on the absorbance readings of an IL-1β/TNF-α/TL-6 standard curve.

The presence of cartilage degradative enzyme MMP-13 was measured using the Rat Matrix Metallo-Proteinase 13 (MMP-13) ELISA kit from Mybiosource (product #: MBS702112 for MMP-13) as follows: serum was added to a microplate coated with MMP-13 antibody and allowed to bind for 2 h at 37° C. The samples were removed and then a biotin-conjugated MMP-13 antibody was added and allowed to bind for 1 h at 37° C. The microplate was thoroughly washed, and an avidin-conjugated Horse Radish Peroxidase was added and allowed to bind for 1 h at 37° C. Enzyme substrate was then added, and the plate was developed for 30 min at 37° C. After the addition of stop solution, the absorbance was read at 450 nm, multiplied by dilution factor, and the concentration of MMP-13 calculated based on the absorbance readings of an MMP-13 standard curve

An increased production of catabolic cytokines is the integral part of collagen-induced arthritis pathology. Rats treated with AMK composition showed a statistically significant reduction in serum IL-1β level when compared to the vehicle-treated CIA group (Table 36). Similarly, marked decreases in serum TNF-α and IL-6 levels were observed for CIA rats treated with AMK or Methotrexate. As depicted in Table 36, significant increases in serum catabolic cytokines IL-1β and IL-6 level were observed for the vehicle-treated CIA group compared to the normal control. AMK-treated rats showed a statistically significant reduction in serum IL-1β (67.4% inhibition, compared to diseased control), IL-6 (60.2% inhibition, compared to diseased control) and TNF-α (75.5% inhibition, compared to diseased control) levels when compared to vehicle-treated diseased rats (Table 36). Methotrexate-treated rats showed reduced serum IL-1β (71.5% inhibition, compared to diseased control), IL-6 (78.6% inhibition, compared to diseased control) and TNF-α (86.2% inhibition, compared to diseased control) levels when compared to vehicle-treated diseased rats (Table 36). This example clearly demonstrated that the natural AMK composition is capable of reducing the catabolic processes of arthritic animals.

Similarly, a marked increase in the level of serum MMP-13 level was observed for the vehicle-treated arthritis diseased rats, when compared to the normal control rats. As seen in Table 36, CIA rats treated with AMK showed a statistically significant reduction in catabolic cartilage degradative enzyme MMP-13 level compared to vehicle-treated CIA rats. This 81.4% inhibition of MMP-13 from AMK-treated CIA rats was calculated as statistically significant vs. CIA-treated rats. Though it was not statistically significant, there was a noteworthy reduction (78.6% compared to vehicle-treated CIA rats) in serum MMP-13 level for the positive drug control Methotrexate-treated rats.

TABLE 36
Effect of Alpinia:Magnolia:Kochia (AMK)
on proinflammatory cytokines in CIA rats

| | IL-1β | IL-6 | TNF-α | MMP-13 |
|---|---|---|---|---|
| Group | (pg/mL) | (pg/mL) | (pg/mL) | (pg/mL) |
| Normal Control | −4.60 ± 1.85*** | 8.02 ± 1.85*** | 3.85 ± 1.18 | 14.96 ± 2.03 |
| CIA+ | 16.91 ± 3.15 | 47.73 ± 8.28 | 2.69 ± 0.90 | 45.13 ± 2.06 |
| MTX (75 μg/mL) | 4.60 ± 5.87* | 10.23 ± 7.37*** | 0.37 ± 1.12 | 9.67 ± 1.80 |
| AMK (200 mg/kg) | 5.52 ± 3.25** | 18.99 ± 8.06** | 0.66 ± 0.94* | 8.38 ± 1.86* |

P ≤ 0.05 vs. Vehicle;
**P ≤ 0.01 vs Vehicle;
***P ≤ 0.001 vs Vehicle

### Example 48. Reduced Cartilage Degradation and Increased Cartilage Regeneration/Rebuilding Activity of Alpinia:Magnolia:Kochia (AMK) in CIA Rats

The presence of the cartilage degradation biomarker uCTX-II was measured using the Rat CTX-II ELISA kit from Mybiosource (product #: MBS2880519) as follows: diluted urine was added to a microplate coated with CTX-II antibody and allowed to bind for 2 h at 37° C. A biotin-conjugated antibody against CTX-II was then added and allowed to bind to the CTX-II from the rat urine for 1 h at 37° C. The microplate was washed thoroughly to remove unbound urine and antibody before an enzyme-conjugated avidin antibody was added to bind to the biotin-conjugated antibody for specific detection. The avidin antibody was allowed to bind for 1 h at 37° C. Washing was repeated, enzyme substrate was added, and the plate was developed for 30 min at 37° C. After the addition of stop solution, the absorbance was read at 450 nm, multiplied by dilution factor, and the concentration of CTX-II calculated based on the absorbance readings of a CTX-II standard curve. CTX-II amount was normalized to the amount of Creatinine in the urine using the Creatinine Parameter Assay Kit from R and D Systems (product #: KGE005) as follows: urine was diluted 1:20, mixed with alkaline picrate (5 parts 0.13% picric acid: 1 part 1 N NaOH) in a microplate and incubated at room temp for 30 min. Absorbance was read at 492 nm and Creatinine amount in urine was calculated based on the absorbance readings of a Creatinine standard curve.

The presence of cartilage regeneration/rebuilding biomarker PIIANP was measured using the Rat Procollagen Type IIA N-Prop (PIIANP) ELISA kit from Mybiosource (product #: MBS9399069) as follows: synovial fluid was added to a microplate coated with PIIANP antibody as well as an HRP-conjugated PIIANP antibody and allowed to bind for one hour at 37° C. The microplate was thoroughly washed, and a Chromagen solution was added and allowed to bind for 15 minutes at 37° C. After the addition of stop solution, the absorbance was read at 450 nm and the concentration of PIIANP calculated based on the absorbance readings of a PIIANP standard curve.

Significant urine CTX-II level changes both in the arthritis disease model and treatment groups were observed. As provided in Table 37, a statistically significant increase in urine CTX-II level was observed for the vehicle-treated CIA group compared to the normal control animals confirming the increased catabolic processes of diseased animals. A higher level of urinary CTX-II is a sign of cartilage degradation, which was significantly inhibited by the composition AMK. Treatment with AMK spared significant degradation of cartilage (inhibited up to 36.7%) compared to vehicle-treated diseased CIA rats. The positive control Methotrexate showed 26.4% inhibition of the cartilage degradation biomarker compared to CIA with p=0.06.

Similarly, the anabolic effect from AMK composition was confirmed by measuring cartilage synthesis/regeneration/rebuilding biomarker—synovial PIIANP. As seen in Table 37, below, a statistically significant increase in synovial PIIANP was observed for the rats treated with AMK when compared to vehicle-treated CIA rats. Rats in this AMK-treated group showed a 79.4% increase in cartilage synthesis/regeneration/rebuild biomarker—synovial PIIANP when compared to vehicle-treated CIA rats. The Methotrexate-treated rats showed 69.8% increase in cartilage repair compared to vehicle-treated CIA rats. In contrast, the vehicle-treated CIA rats experienced more than a 19-fold decrease in the level of PIIANP indicating the shift of arthritic animals more towards cartilage degradation than repair.

TABLE 37
Anti-catabolic and anabolic activities of AMK in CIA rats

| | CTX-II | PIIANP |
|---|---|---|
| Group | (ng/g creatinine) | (ng/mg) |
| Normal Control | 2207.2 ± 264.0 | 2.52 ± 0.56*** |
| CIA+ | 3396.9 ± 274.8 | 0.13 ± 0.12 |
| MTX (75 μg/mL) | 2500.8 ± 355.9 | 0.43 ± 0.16 |
| AMK (200 mg/kg) | 2152.3 ± 246.9*** | 0.63 ± 0.13* |

P ≤ 0.05 vs. Vehicle;
**P ≤ 0.01 vs Vehicle;
***P ≤ 0.001 vs Vehicle

### Example 49. Histopathology Findings for CIA Rats Treated with Alpinia:Magnolia:Kochia (AMK)

For histopathological examination, the ankle joints were kept in 10% formalin for 72 h. The fixed specimens were then decalcified with Calci-Clear Rapid for one and a half days and embedded in paraffin. Standardized 5 μm serial sections were obtained at the medial and lateral section in the sagittal plane of the joint and were stained with hematoxylin and eosin (HE) and Safranin O-fast green to enable evaluation of proteoglycan content. A modified Mankin system (Mankin et al., 1971) was used to score structural and cellular alterations of joint tissues resulting from disease progression and/or treatment efficacy. The histological analysis was conducted at Nationwide Histology and slides were examined by a certified pathologist.

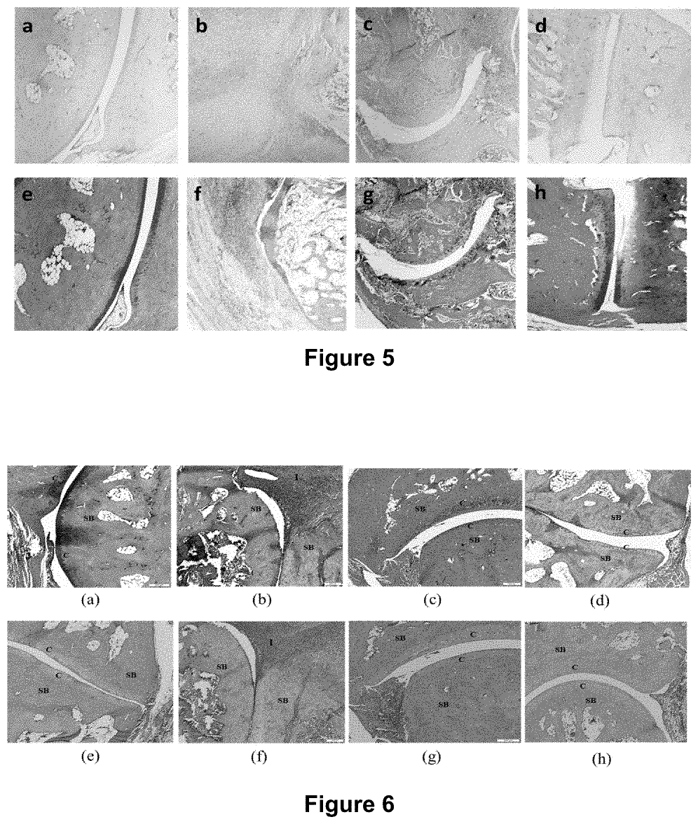

The histopathology data was in alignment with the severity score of arthritis. When compared to the normal control rats, vehicle-treated rats showed severe synovitis, marked cartilage degradation, synovial hyperplasia, pannus formation, and bone erosion (FIG. 5, Table 38). These changes for the vehicle-treated CIA rats in comparison to the normal controls were reflected as 3.3-fold, 3.8-fold, 4.7-fold and 24.2-fold increase in cartilage degradation, GAG loss, bone erosion and inflammation, respectively. In contrast, rats treated with AMK had nearly normal morphology, with minimal alterations in matrix integrity, a smoother articulation cartilage surface, low levels of mononuclear cell infiltration, and synovial hyperplasia, as well as reduced articular bone damage. Cartilage protection and hence anti-catabolic activity of composition AMK was found to be 65.1% with a 61.9% maintenance of matrix integrity in comparison to the vehicle-treated CIA rats. In agreement with the biomarker data (reduced catabolic cytokines IL-1β, TNF-α and IL-6), there was an 88.2% reduction in inflammation in composition AMK-treated rats when compared to vehicle-treated CIA rats. There was also a 73.1% reduction in bone erosion for the CIA rats treated with AMK composition. These changes were statistically significant for each parameter monitored compared to vehicle-treated CIA rats.

TABLE 38
Histopathology findings in CIA rats
treated with Alpinia:Magnolia:Kochia (AMK)

| | Cartilage | | Bone | |
|---|---|---|---|---|
| | destruction | GAG Loss | Erosion | Inflammation |
| Group | (Mean ± SE) | (Mean ± SE) | (Mean ± SE) | (Mean ± SE) |
| Normal | 1.29 ± 0.28* | 1.08 ± 0.24* | 0.71 ± 0.27** | 0.21 ± 0.12** |
| Control | (70%) | (74.0%) | (78.8%) | (95.9%) |
| CIA+ | 4.30 ± 0.84 | 4.15 ± 0.85 | 3.35 ± 0.51 | 5.08 ± 0.95 |
| MTX | 2.90 ± 0.63 | 2.04 ± 0.62* | 1.13 ± 0.46* | 3.25 ± 1.02 |
| (75 μg/mL) | (32.6%) | (50.8%) | (66.3%) | (36.0%) |
| AMK | 1.50 ± 0.17* | 1.58 ± 0.49* | 0.90 ± 0.29* | 0.60 ± 0.35* |
| (200 mg/kg) | (65.1) | (61.9%) | (73.1%) | (88.2%) |

*P ≤ 0.05 vs Vehicle;
** P ≤ 0.01 vs Vehicle;
data in the parenthesis is percent inhibition against vehicle treated disease model.
Cartilage destruction (0-6): Cartilage thickness/thinning, irregular surface frayed/fissure loss, degeneration, ulcerative necrosis/fragmentation, severe disorganization/chaotic;
Bone damage (0-6): Subchondral bone thickness/volume and density, osteoclastic activity, subchondral bone damage;
Inflammation/Cellular infiltration (0-6): Cellular Infiltration/Inflammation and Proliferation, hypercellular, cluster/hypocellular;
Matrix GAGs loss (0-6): Matrix GAG reduction: radial, interterritorial to pericellular loss of staining, femoral condyle/tibial plateau integrity, and thickness of articular Cartilage.
Data expressed as Mean ± SE.

FIG. 5 shows histopathology images (HE a-d and Safranin O e-f) from ankle joint of CIA induced rats treated with AMK and MTX. A and e—normal control, b and f—CIA+vehicle, c and g—CIA+MTX, d and h—CIA+AMK.

Following documentation of the above Alpinia:Magnolia:Kochia (AMK) efficacy outcomes from the CIA model as described from Examples 40-49 here in the current subject matter, a dose-response study for AMK in the CIA model was carried on to extrapolate an optimum human equivalent dose conversion as suggested by the FDA (http://www.fda.gov/cder/guidance/index.htm). In this FDA guidance for the industries, a 0.16 conversion factor has been suggested for a rat dosage in mg/kg to human (i.e. rat dose in mg/kg multiply by 0.16=human equivalent dose in mg/kg). As such, in this CIA study rats are being administered at oral doses of 40, 60, 80 and 120 mg/kg/day of AMK for 5 weeks. These dosages would give a human equivalent dose of 448, 672, 896 and 1344 mg/day for an average 70 kg adult. We believe that results from this dose-response study would provide a basis for future human clinical trial dosage determination.

### Example 50. Second Collagen-Induced Arthritis Model Induction and AP Treatment

Male Sprague Dawley rats (7-8 weeks old, n=40) were purchased from Charles River Laboratories Inc. (Wilmington, Mass., USA) and acclimated upon arrival for two weeks before being assigned randomly to their respective treatment groups: G1=Normal control (−) (n=10/group), G2=collagen-induced arthritis (CIA)+Vehicle (0.5% Carboxy Methylcellulose) (n=10/group), G3=CIA+Methotrexate (+) (0.5 mg/kg) (n=10/group), and G4=CIA+AP (+) (200 mg/kg) (n=10/group). Treatment was initiated two weeks before model induction and lasted for an additional three weeks thereafter. Collagen type-II (Lot #845) from bovine nasal septum and Incomplete Freund's adjuvant (IFA) (Lot #SLBR0642v) were purchased from Elastin Products Company (Owensville, Mich., USA) and Sigma (St. Louise, Mo., USA), respectively. All materials were kept at suitable temperatures as recommended by the manufacturer. At the time of preparation, 60 mg of collagen was weighed and added to pre-chilled 15 mL 0.1 M acetic acid in a 60 mL-sized flask with a magnetic stirrer to yield 4 mg/mL concentration (Brand, et al., 2003; Rosloniec et al., 2001). The mixture was dissolved by gently stirring overnight at 4° C. The next morning, the dissolved collagen was emulsified with equal volume of IFA (15 mL) to achieve a final concentration of 2 mg/mL collagen. Rats sedated with isoflurane were then primed intradermally with 400 μL of the emulsified collagen at the base of their tail at two sites using a 1 mL syringe fitted with a 26 G needle. The dissolved mixture was kept in an ice bucket and stirred between groups at the time of injection to preserve uniform consistency. On the seventh day, rats were inoculated with a booster dose of 2 mg/mL type II collagen emulsified with equal volume of incomplete adjuvant at 100 μL/rat/site.

Clinical findings such as arthritis severity index, paw thickness, ankle diameter (using Digital Absolute, Model #PK-0505CPX, Mitutoyo Corporation, Kawasaki, Japan), and pain sensitivity (using Randall Salitto, IITC Life Science Inc., Woodland Hills, Calif., USA) were monitored during the course of study. Urine was collected from overnight fasted rats using metabolic cages after three weeks of treatment post-model induction. At Necropsy, serum from the cardiac and synovial lavage (100 μL of saline was injected into the articular cavity and aspirated back to the syringe) for biomarkers and ankle joint for histopathology were collected from each animal.

Linear trapezoid rule was used to calculate the area under the curve (AUC) for days 9-21.

% Inhibition={(Mean value of treatment-mean value of CIA+)/(Mean value of control-mean value of CIA)}*100.

### Example 51. Reduction of Arthritis Severity Index by Composition AP in CIA Rats

Rats continued to show a slow progression of disease for the duration of study. As seen in the data below, rats treated with both the treatment groups such as Methotrexate and AP showed statistically significant suppression in arthritis severity from day 12 and continued this significance for the duration of study (Table 39).

At the end of the study, average severity scores of 3.5±0.42, 1.1±0.17, and 2.0±0.89, were observed for rats treated with Vehicle, Methotrexate and AP, respectively. These values demonstrated a clear effect and potency of AP and drug treatments in relation to the vehicle-treated disease model. When the area under the arthritis severity curve was calculated, the percent reductions of 78.8% (p=0.002) and 54.9% (p=0.02) with statistical significance were observed from positive drug control Methotrexate and AP treatment, respectively. (Table 39).

### Example 52. Reduction of Ankle Diameter by Composition AP in CIA Rats

Statistically significant reduction in ankle diameter was observed for rats treated with Methotrexate and AP until day 16 post induction (Table 40). Thereafter, only the Methotrexate group showed a statistically significant reduction in ankle diameter. Only Methotrexate group showed a statistically significant reduction (i.e. 93.6%) in ankle width when the area under the curve was considered for days 9 to 20. A statistically non-significant 60.6% reduction in ankle diameter were observed for AP-treated CIA rats for the AUC (Table 40).

TABLE 40
Changes in ankle diameter for CIA rats treated with AP composition

| | | Ankle diameter | | | | | | |
|---|---|---|---|---|---|---|---|---|
| Groups | Stat | Priming | Boost | Day 12 | Day 14 | Day 16 | Day 18 | Day 20 |
| NC | Mean | 6.02 | 6.11 | 6.13** | 6.06*** | 6.10*** | 6.08*** | 6.03*** |
| | Sd | 0.08 | 0.04 | 0.04 | 0.03 | 0.01 | 0.02 | 0.05 |
| CIA+ | Mean | 6.10 | 6.11 | 6.50 | 7.43 | 8.07 | 8.56 | 8.94 |
| | Sd | 0.08 | 0.07 | 0.34 | 0.59 | 0.21 | 1.18 | 1.07 |
| MTX (0.5 mg/kg) | Mean | 0.10 | 0.07 | 0.03** | 0.04*** | 0.07*** | 0.08*** | 0.08*** |
| | Sd | 0.10 | 0.06 | 0.11 | 0.22 | 0.46 | 0.71 | 1.00 |
| AP (200 mg/kg) | Mean | 6.09 | 6.00 | 6.05** | 6.30*** | 6.59*** | 7.60 | 7.84 |
| | Sd | 0.06 | 0.12 | 0.04 | 0.33 | 0.35 | 1.12 | 1.40 |

**P ≤ 0.001; ***P ≤ 0.0001; MTX: Methotrexate

TABLE 39
Changes of Arthritis severity index for rats treated with AP in CIA model

| | | | | Arthritis Index | | | | | | | | | |
|---|---|---|---|---|---|---|---|---|---|---|---|---|---|
| | | Inductions | | Day | Day | Day | Day | Day | Day | Day | Day | Day | Day |
| Groups | Stat | Priming | Boost | 10 | 11 | 12 | 13 | 14 | 15 | 16 | 17 | 19 | 20 |
| NC | Mean | 0.00 | 0.00 | 0.00 | 0.00 | 0.00** | 0.00** | 0.00** | 0.00** | 0.00** | 0.00** | 0.00** | 0.00** |
| | Sd | 0.00 | 0.00 | 0.00 | 0.00 | 0.00 | 0.00 | 0.00 | 0.00 | 0.00 | 0.00 | 0.00 | 0.00 |
| CIA+ | Mean | 0.00 | 0.00 | 0.08 | 0.23 | 0.75 | 1.25 | 2.1 | 2.875 | 3.375 | 3.725 | 3.825 | 3.6 |
| | Sd | 0.00 | 0.00 | 0.11 | 0.28 | 0.45 | 0.55 | 0.60 | 0.32 | 0.36 | 0.31 | 0.35 | 0.45 |
| MTX | Mean | 0.00 | 0.00 | 0 | 0 | 0.05** | 0** | 0** | 0.35** | 0.875** | 1** | 1.075** | 1.1** |
| (0.5 mg/ | Sd | 0.00 | 0.00 | 0.00 | 0.00 | 0.15 | 0.00 | 0.00 | 0.23 | 0.20 | 0.00 | 0.16 | 0.17 |
| kg) | | | | | | | | | | | | | |
| AP(200 | Mean | 0.00 | 0.00 | 0 | 0 | 0.1* | 0.25** | 0.55** | 1.05** | 1.6** | 1.9** | 1.95* | 2.23** |
| mg/kg) | Sd | 0.00 | 0.00 | 0.00 | 0.00 | 0.17 | 0.35 | 0.52 | 0.56 | 0.59 | 0.80 | 0.83 | 1.00 |

*P ≤ 0.001; **P ≤ 0.0001

### Example 53. Reduction of Paw Thickness by Composition AP in CIA Rats

In agreement with the severity score and ankle diameter, rats treated with Methotrexate and AP showed a statistically significant reduction in paw swelling starting from day 12 and maintained this significance for the duration of study (Table 41). However, when the total area under the swelling curve (day 7-day 20) for this reduction was considered, rats only in the Methotrexate group showed statistically significant 93.8% reduction in paw edema compared to the vehicle-treated CIA group (Table 41). A percent reduction of 69.3% in paw edema with P-value of 0.058, was observed for rats treated with AP composition, compared to the vehicle-treated CIA rats (Table 41)

TABLE 41
Changes in paw thickness for CIA rats treated with composition AP

| | Dose | Paw thickness | | | | | | |
|---|---|---|---|---|---|---|---|---|
| Groups | (mg/kg) | Priming | Boost | Day 12 | Day 14 | Day 16 | Day 18 | Day 20 |
| Normal | 0 | 2.96 ± 0.08 | 2.94 ± 0.07 | 2.94 ± 0.07* | 3.00 ± 0.05† | 3.07 ± 0.04† | 3.03 ± 0.03† | 2.93 ± 0.29† |
| control | | | | | | | | |
| CIA+ | 0 | 2.99 ± 0.04 | 2.95 ± 0.04 | 3.49 ± 0.43 | 4.62 ± 0.57 | 5.59 ± 0.31 | 6.12 ± 0.50 | 6.01 ± 0.48 |
| MTX | 0.5 | 2.93 ± 0.07 | 2.95 ± 0.06 | 2.99 ± 0.06** | 3.01 ± 0.03† | 3.21 ± 0.05† | 3.25 ± 0.05† | 3.23 ± 0.06† |
| AP | 200 | 2.92 ± 0.03 | 2.89 ± 0.02 | 2.99 ± 0.09** | 3.45 ± 0.61† | 3.77 ± 0.59† | 4.14 ± 0.81† | 4.08 ± 0.83† |

*P ≤ 0.05; **P ≤ 0.001; †P ≤ 0.0001

### Example 54. Arthritis Index, Ankle Diameter and Area Under the Response Curve (AUC) for Paw Thickness of CIA Rats Treated with Composition AP

As seen in Table 42, below, rats treated with AP showed 69.3%, 60.7% and 54.9% inhibition in paw thickness, ankle diameter and arthritis severity index during the course of the study period when compared to vehicle treated CIA rats, respectively. These reductions were more than 50% for each parameter, indicating the significance of composition AP in reducing arthritis-associated symptoms. In comparison, the Methotrexate-treated rats showed 93.8%, 93.6% and 78.8% reduction in paw thickness, ankle diameter and arthritis severity index, respectively.

### Example 55. Reduction of the Compression-Induced Pain by Composition AP in CIA Rats, Indicating its Symptom Relief Activity

Response to pressure as a measure of pain sensitivity was measured using the Randall-Salitto probe attached to an electronic monitor on priming day, boost day, and days 12, 14, 16, 18 and 20. Both the left and right hind legs were monitored on those days and their averages were used for data analysis. Changes from the vehicle-treated CIA rats have been reported as pain tolerance on those days. The highest pain tolerance was observed for rats in the Methotrexate group (69.4% improvement on day 20) followed by the AP (31.5% improvement on day 18) (Table 43). This reduction in pain sensitivity was statistically significant at all time points as of day-12 for both groups when compared to vehicle-treated CIA rats (Table 43). Rats treated with composition AP showed ranges of 8.25-31.5% reduction in pain sensitivity compared to vehicle treated CIA rats. The Methotrexate group showed 8.39-69.4% reduction in pain sensitivity in the same duration.

### Example 56. Catabolic Cytokines, Matrix Degrading Enzymes and Cartilage Synthesis Biomarker Changes by Composition Alpinia:Pepper (AP) in CIA Rats

At the completion of the study, blood from cardiac puncture was collected from each animal. Blood was spun at 3000 rpm for 15 min. About 700-800 μl of serum was isolated from each rat. Samples were kept at −80° C. until use.

The presence of catabolic cytokines IL-6/TNF-α was measured using the Rat IL-6/TNF-α Quantikine ELISA kit from RandD Systems as follows: undiluted serum was added to a microplate coated with polyclonal IL-6/TNF-α antibody and allowed to bind for 2 hours at room temperature. The microplate was washed thoroughly to remove unbound serum, and then a polyclonal enzyme-conjugated IL-6/TNF-α antibody was added and allowed to bind for 2 hours at room temperature. Washing was repeated, enzyme substrate was added, and the plate was developed for 30 minutes at room temperature. After the addition of stop solution, the absorbance was read at 450 nm and the concentration of IL-6/TNF-α calculated based on the absorbance readings of an IL-6/TNF-α standard curve.

TABLE 42
Area under the response curve for CIA rats treated with Alpinia:Pepper (AP)

| | AUC_{(d9-d21)} | | | | | | | | |
|---|---|---|---|---|---|---|---|---|---|
| | (Mean ± SD) | | | % Inhibition | | | P-values VS CIA + vehicle | | |
| | Paw | Ankle | Arthritis | Paw | Ankle | Arthritis | Paw | Ankle | Arthritis |
| Group | thickness | diameter | index | thickness | diameter | index | thickness | diameter | index |
| CIA+ | 24.30 ± 0.75 | 38.08 ± 0.99 | 23.56 ± 1.44 | — | — | — | — | — | — |
| MTX | 15.55 ± 0.21 | 30.94 ± 0.11 | 5.00 ± 0.48 | 93.81 | 93.59 | 78.78 | 0.01 | 0.01 | 0.002 |
| (0.5 mg/kg) | | | | | | | | | |
| AP | 17.84 ± 0.31 | 33.45 ± 0.71 | 10.63 ± 0.85 | 69.26 | 60.65 | 54.90 | 0.06 | 0.12 | 0.02 |
| (200 mg/ | | | | | | | | | |
| kg) | | | | | | | | | |

TABLE 43
Anti-pain activity of Alpinia:Pepper (AP) in CIA rats

| | Compression pain resistance vs CIA + vehicle | | | | | | |
|---|---|---|---|---|---|---|---|
| | (Mean ± SD) | | | | | | |
| Group | Prime | Boost | Day 12 | Day 14 | Day 16 | Day 18 | Day 20 |
| Normal | −3.58 ± | −1.66 ± | 15.47 ± | 33.21 ± | 71.51 ± | 89.73 ± | 83.96 ± |
| control | 3.97† | 1.92† | 1.58† | 1.66† | 2.59† | 1.61† | 1.19† |
| MTX | −1.20 ± | −1.50 ± | 8.39 ± | 27.30 ± | 54.31 ± | 64.13 ± | 69.35 ± |
| (0.5 | 2.84† | 1.72† | 2.25† | 1.59† | 2.56† | 2.18† | 1.51† |
| mg/kg) | | | | | | | |
| AP | −0.39 ± | −0.68 ± | 8.25 ± | 19.30 ± | 20.39 ± | 31.48 ± | 28.97 ± |
| (200 | 2.54† | 2.12† | 1.68† | 1.54† | 2.64† | 5.19† | 2.81† |
| mg/kg) | | | | | | | |

†P ≤ 0.0001 vs vehicle treated CIA

The presence of cartilage degradative enzyme MMP-13 was measured using the Rat Matrix Metalloproteinase 13 (MMP-13) ELISA kit from Mybiosource (product #: MBS702112) as follows: undiluted serum was added to a microplate coated with MMP-13 antibody and allowed to bind for 2 hours at 37° C. The samples were removed, and then a biotin-conjugated MMP-13 antibody was added and allowed to bind for 1 hour at 37° C. The microplate was thoroughly washed, and an avidin-conjugated Horse Radish Peroxidase was added and allowed to bind for 1 hour at 37° C. Enzyme substrate was then added, and the plate was developed for 30 minutes at 37° C. After the addition of stop solution, the absorbance was read at 450 nm and the concentration of MMP-13 calculated based on the absorbance readings of an MMP-13 standard curve.

The level of cartilage regeneration biomarker PIIANP was measured using the Rat Procollagen Type IIA N-Prop (PIIANP) ELISA kit from Mybiosource (product #: MBS9399069) as follows: undiluted serum was added to a microplate coated with PIIANP antibody as well as an HRP-conjugated PIIANP antibody and allowed to bind for one hour at 37° C. The microplate was thoroughly washed, and a Chromagen solution was added and allowed to bind for 15 minutes at 37° C. After the addition of stop solution, the absorbance was read at 450 nm and the concentration of PIIANP calculated based on the absorbance readings of a PIIANP standard curve.

Composition AP administered orally at 200 mg/kg for 3 weeks, significantly reduced the serum catabolic biomarkers IL-6, TNF-α and MMP-13 levels when compared to vehicle-treated CIA rats. The most significant inhibition in proinflammatory and catabolic cytokines as a result of composition AP treatment was observed in IL-6, which was reduced by 58.7% in comparison to the vehicle-treated CIA rat group. This reduction was complemented by a 43.5% reduction in matrix degrading enzyme MMP-13 for the AP-treated rats. The IL-6 and MMP-13 data seem a true reflection of what was observed in the in-life study as far as clinical measurements and histopathology findings for the AP-treated rats. The positive drug control Methotrexate-treated rats experienced significantly reduced amounts of IL-6 and TNF-α in the serum.

A statistically significant decrease in serum cartilage regeneration biomarker PIIANP was observed for the CIA rats treated with vehicle compared to the control group (p=0.0017) (Table 44). The positive control Methotrexate had a significant increase in serum PIIANP (47%, p=0.0017 compared to CIA+vehicle). AP administered orally at 200 mg/kg for 3 weeks, showed an increase in serum cartilage regeneration biomarker PIIANP (i.e. 18%), but the increase was not significant. These results indicated that the AP treatment still shifted the progression of arthritic rats toward cartilage regeneration/rebuilding, though to a lesser extent than the drug control. This shows that AP and drug treatments contribute to the reversal of the collagen degradation phenotype that is characteristic of this arthritic animal model.

TABLE 44
Catabolic Pathways down regulated by Alpinia:Pepper (AP) in CIA rats

| | PIIANP | IL-6 | TNF-α | MMP-13 |
|---|---|---|---|---|
| Group | (ng/mL) | (pg/mL) | (pg/mL) | (pg/mL) |
| Normal Control | 6.08 ± 0.75** | 14.62 ± 7.3** | 9.57 ± 0.3*** | 87.19 ± 40.81 |
| CIA+ | 4.69 ± 0.91 | 35.83 ± 18.06 | 10.68 ± 0.38 | 85.09 ± 34.14 |
| MTX | 6.53 ± 1.4** | 3.33 ± 6.44*** | 10.2 ± 0.37* | 86.57 ± 45.31 |
| (0.5 mg/mL) | | | | |
| AP (200 mg/kg) | 5.44 ± 0.66 | 14.8 ± 17.08* | 10.09 ± 0.77* | 48.13 ± 14.43** |

*P ≤ 0.05 vs Vehicle;
**P ≤ 0.001 vs vehicle;
***P ≤ 0.0001 vs Vehicle

### Example 57. Changes in Histopathology Readings by Composition Alpinia:Pepper (AP) in CIA Rats

At necropsy, the ankle joint was carefully dissected out, fixed in 10% buffered formalin and sent to Nationwide Histology (Veradale, Wash., USA) for further histopathology analysis. The fixed specimens were then decalcified with Calci-Clear Rapid for one and a half days and embedded in paraffin. Standardized 5 m serial sections were obtained from each rat and stained with hematoxylin and eosin (HE) and Safranin O-fast green to enable evaluation of proteoglycan content. A modified Mankin system (Mankin et al., 1981) was used to score structural and cellular alterations of articular components as indications of disease progression and/or treatment efficacy. The histological analysis was conducted by a certified Pathologist at Nationwide Histology.

The histopathology data were in alignment with the severity scores of arthritis. When compared to normal control rats, vehicle-treated CIA rats showed severe synovitis, marked cartilage degradation, synovial hyperplasia, pannus formation and bone erosion (FIG. 5 and Table 45). These changes for the vehicle-treated CIA rats in comparison to the normal controls were reflected as 17.9-fold, 7.9-fold, 181-fold and 52.7-fold increases in cartilage degradation, GAG loss, bone erosion and inflammation, respectively. In contrast, rats treated with Methotrexate had nearly normal morphology with minimal alternation in matrix integrity, a smoother articulation cartilage surface, low levels of mononuclear cell infiltration and synovial hyperplasia, as well as reduced articular bone damage (Table 45). Similarly, rats treated with AP composition also showed statistically significant reductions in cartilage destruction, inflammation severity, bone erosion and GAG loss compared to CIA rats treated with vehicle. Cartilage protection and hence anti-catabolic activity of composition AP was found to be 57.7% with a 47.5% maintenance of matrix integrity in comparison to the vehicle-treated CIA rats. In agreement with the biomarker data (such as reduced catabolic TNF-α and IL-6), there was a 67.0% reduction in inflammation when compared to vehicle-treated CIA rats. There was also a 61.0% reduction in bone erosion for the CIA rats treated with AP composition. These changes from AP treatment were statistically significant for each parameter monitored compared to vehicle-treated CIA rats.

TABLE 45
Histopathology findings in CIA rats treated
with composition Alpinia:Pepper (AP)

| | Cartilage | | | |
|---|---|---|---|---|
| | destruction | GAG Loss | Bone Erosion | Inflammation |
| Group | (Mean ± SE) | (Mean ± SD) | (Mean ± SE) | (Mean ± SE) |
| Normal | 0.42 ± 0.15*** | 0.50 ± 0.35*** | 0.04 ± 0.04*** | 0.17 ± 0.05*** |
| Control | (94.4%) | (87.4%) | (99.4%) | (98.1%) |
| CIA+ | 7.50 ± 0.68 | 3.96 ± 0.88 | 7.25 ± 0.60 | 8.96 ± 0.34 |
| MTX | 0.13 ± 0.05*** | 0.21 ± 0.17*** | 0.00 ± 0.00*** | 0.00 ± 0.00*** |
| (0.5 mg/mL) | (98.3%) | (94.5%) | (100%) | (100%) |
| AP | 3.17 ± 1.44* | 2.08 ± 1.58* | 2.83 ± 1.65* | 2.96 ± 1.64* |
| (200 mg/kg) | (57.7%) | (47.5%) | (61.0%) | (67.0%) |

*P ≤ 0.05 vs Vehicle;
***P ≤ 0.00001 vs Vehicle;
data in the parenthesis is percent inhibition against vehicle treated disease model.
Data in the parenthesis is percent inhibition against vehicle treated disease model.
Cartilage destruction (0-6): Cartilage thickness/thinning, irregular surface frayed/fissure loss, degeneration, ulcerative necrosis/fragmentation, severe disorganization/chaotic;
Bone damage (0-6): Subchondral bone thickness/volume and density,
osteoclastic activity, subchondral bone damage;
Inflammation/Cellular infiltration (0-6): Cellular Infiltration/Inflammation and Proliferation, hypercellular, cluster/hypocellular;
Matrix GAGs loss (0-6): Matrix GAG reduction: radial, interterritorial to pericellular loss of staining, femoral condyle/tibial plateau integrity, and thickness of articular Cartilage.
Data expressed as Mean ± SE.
Figure 6 shows RE and Safranin 0 staining histology for CIA rats treated with AP (RE stains (40x):
a = normal control + Vehicle,
b = CIA + Vehicle,
c = CIA + Methotrexate,
d = CIA + AP,
Safranin 0 stain (40x):
e = normal control + vehicle,
f = CIA + Vehicle,
g = CIA + Methotrexate,
h = CIA + AP,
C = cartilage,
SB = subchondral bone,
I = inflammation)

### Example 58. Implications of the Cartilage Protection and Symptom Relief Activities of Alpinia:Pepper (AP) and Alpinia:Magnolia:Kochia (AMK) in Collagen-Induced Arthritis (CIA)

The CIA model in rats is the most commonly studied autoimmune model of RA with several pathological features resembling the immune-mediated polyarthritis in humans (Miyoshi et al., 2018). Its short duration between immunization and disease manifestation makes the model feasible for therapeutic efficacy evaluations. Following inoculation of heterogenic type II collagen (CII), rats mount both humoral and cellular responses to the antigen (Brand et al., 2003). This sensitization subsequently leads to the host animal attacking its own type II collagen, which is predominantly present in the joint cartilage and hence results in erosive or non-erosive joint destruction. The pathophysiology of the disease is highly orchestrated and complex. Upon induction, rats experience inflammatory pain and swelling, cartilage degradation, synovial hyperplasia, pannus formation, mononuclear cell infiltration, deformity, and immobility.

In the CIA studies described herein, rats started to show the pathognomonic signs of arthritis on day 9 post-priming followed by a progressive increase in severity that approached near plateau on days 19 to 21. These symptoms were mitigated by oral treatment of an immune suppressant-Methotrexate and also the novel natural compositions—Alpinia:Pepper (AP) and Alpinia:Magnolia:Kochia (AMK). All treatment groups (Methotrexate, AP and AMK) showed measurable relief in arthritis severity, swelling, ankle width, and pain sensitivity when compared to the vehicle-treated diseased rats. When data for arthritis severity, paw thickness, and ankle diameter were pooled together for the duration of the study period from day 10 to 21 (where visible signs of arthritis were observed), CIA rats treated with Methotrexate, AMK and AP showed statistically significant reductions in all of the cardinal signs of arthritis suggesting their application for symptomatic relief of arthritis.

Catabolic TNF-α and IL-1β are the two primary cytokines involved in the initiation and progression of arthritis (Kapoor et al., 2011), mainly through (a) inhibition of anabolic activities of chondrocytes, leading to downregulation of extracellular matrix component biosynthesis, (Saklatvala et al., 1986; Goldring et al., 1994); (b) induction of additional catabolic cytokines (such as IL-6), chemokines, and extracellular matrix degrading enzymes (MMPs and aggrecanases) (Lefebvre et al., 1990; Guerne et al., 1990); (c) inhibition of anti-oxidant activity of the host (Mathy-Hartert,et al., 2008); and (d) induction of reactive oxygen species (Lepetsos et al., 2016).

These processes facilitate maintenance of the catabolic processes of arthritis, indicated by chronic inflammation and perpetual joint destruction in arthritic patients. For example, while injection of IL-1β into the knee joints of rats caused joint inflammation and marked proteoglycan depletion (Chandrasekhar et al., 1992; Bolon et al., 2004), its blockade reversed the catabolic process (Joosten et al., 1999; Kobayashi et al., 2005; van de Loo et al., 1992). Besides direct involvement in the catabolic inflammation process and cartilage degradation, dysregulation of IL-6 levels is also linked to the common clinical manifestations associated with rheumatoid arthritis pathology, such as fever, fatigue, and weight loss (Wei et al., 2015). Hence, modulating these catabolic pro-inflammatory cytokines at various stages of disease progression could shift the balance of arthritis away from catabolic processes while alleviating the symptoms associated with arthritis and/or helping to modify the disease. The regulation of homeostasis of chondrocytes, extracellular matrix, articular cartilage, and the phenotype of arthritis from Alpinia:Pepper (AP) and Alpinia:Magnolia:Kochia (AMK) that was observed in this CIA study and above other examples could be in part due to inhibition of these key catabolic pro-inflammatory cytokines.

Supplementation with AMK for three weeks resulted in a significant reduction in the level of fundamental matrix proteolytic enzymes, such as MMP-13. Along with aggrecan breakdown, degradation of collagen is a central feature or phenotype of arthritis. Pro-inflammatory cytokines, such as TNF-α, IL-1β, and IL-6 are known to play important roles in cartilage matrix degradation in articular cartilage through a cascade of catabolic events that leads to stimulation of aggrecanase and matrix metalloproteinase production (Kapoor et al., 2011). During the course of disease pathology, the major histocompatibility complex presents these fragments to T cells and promotes the activation and release of large amounts of inflammatory cytokines, such as IL-1β and IL-6, which in turn increases expression levels of other MMPs in the chondrocytes and synovial fibroblasts. Consequentially, these catabolic processes result in the phenotype of arthritis with augmented collagenase activity and worsening of joint inflammation. MMP-13 has been found in increased levels at the sites of cartilage erosion in cases of rheumatoid arthritis and osteoarthritis (Rose et al., 2016). Previous studies have shown that these MMP levels in OA patient's blood and synovial fluid were higher than in healthy people and the level was consistent with the extent of cartilage damage (Yamanaka et al., 2000; Galil et al., 2016). In fact, MMPs secreted into the synovial fluid can directly degrade the cartilage and bone composition, leading to enhanced damage of surrounding articular structures (Ma et al., 2015). In our study, there was significant suppression of MMP-13 levels by AMK and AP, which provided protection of cartilage from degradation, improved pain relief and suppression of the phenotype of arthritis. The reduction in MMPs observed in this study could partially be explained by (a) the effect of treatment materials in reducing the catabolic pro-inflammatory cytokines and/or (b) the activity of treatment materials directly suppressing expression of these matrix degrading enzymes.

Urine C-terminal telopeptide of type II collagen (uCTX-II) has been by far the most studied and frequently referred to biomarker of cartilage degradation that could be used for the purpose of diagnosis, determining the severity of disease or extent of disease progression, prognosis, and monitoring efficacy of treatment (Oestergaard et al., 2006). In clinical studies, high levels of uCTX-II are a good predictor of increased risk of joint destruction (Garnero et al., 2001). Degradation and loss of articular cartilage are fundamental phenotypes of arthritis, whereby increased CTX-II levels directly correlated with the time course of paw swelling and arthritis severity indicated by the narrowing of joint space and loss of total cartilage volume. Our results were in accord with previous reports (Oestergaard et al., 2006; Siebuhr et al., 2012). In the current study, substantiating the beneficial effects of Alpinia:Pepper (AP) and Alpinia:Magnolia:Kochia (AMK) on reduction of paw swelling, paw thickness, arthritis severity, and decrease of pro-inflammatory cytokines and matrix degrading enzymes, rats treated with AP and AMK showed significantly reduced levels of uCTX-II. These findings indicated that cartilage protection activity is one of the primary functions of AP and AMK, suggesting their usage in regulating homeostasis of chondrocytes, extracellular matrix, articular cartilage, and phenotype of arthritis.

Together with symptoms and biomarkers, histopathological analyses of articular cartilage, synovial membrane, and subchondral bone have been used to evaluate arthritis disease progression and outcomes of therapeutic interventions (Chen et al., 2017). In these CIA studies, significant improvements in maintenance of the articular structural integrity of rats treated with AMK, AP and Methotrexate were observed. These effects were demonstrated in the histopathology data as exhibited by limited loss, degeneration, or necrosis of chondrocytes, smoother articular cartilage surface, deeper and uniform stain of intracellular matrix, and close to normal contour of the subchondral bone. The changes in magnitude of histopathological severity scores for: 1. cartilage degradation, 2. bone damage, 3. inflammation, and 4. matrix integrity were computed and it was found that AMK and AP treatment resulted in 65.1% and 57.7%, 73.1% and 61.0%, 8.2% and 67.0%, and 61.9% and 47.5% inhibition, respectively, for each outcome, when compared to vehicle-treated CIA rats.

Collectively, in these CIA studies, AMK and AP orally administered produced (a) reduced catabolic inflammation as reflected by reduced arthritis index, paw thickness, paw edema, and reduced catabolic cytokines (IL-1β, IL-6, and TNF-α); (b) decreased pain sensitivity; (c) increased cartilage sparing activity and maintenance of articular structure as indicated by lower uCTX-II and cartilage degrading enzymes (MMP-13) and (d) improved cartilage synthesis and repair (as documented in the increased level of PIIANP). These properties of AMK and AP suggest their potential applications as alternative natural therapies for arthritis management by maintaining the normal homeostasis of cartilage and enhancing anabolic phenotype of arthritis.

### Example 59. Mono-Iodoacetate (MIA)-Induced Experimental Osteoarthritis Model Induction and AMK Treatment

The MIA-induced OA disease model in rats is a standardized model most frequently used to mimic human OA (Lee et al., 2014). The model involves inoculation of MIA into a femorotibial joint pocket that induces pain responses in the ipsilateral limb accompanied by progressive cartilage degradation. Intra-articular injection of MIA disrupts chondrocyte glycolysis by inhibiting glyceraldehyde-3-phosphatase dehydrogenase and results in chondrocyte death, neovascularization, subchondral bone necrosis and collapse, as well as inflammation (Guzman et al., 2003). These phenotypic characteristics make the model very attractive to evaluate compounds for their anti-inflammatory, analgesic and/or potential disease-modifying activities as it shares similar disease pathology to the human OA. As a result, we selected this validated in vivo model to investigate the effect of AMK in mitigating pain sensitivity, regulating the phenotype of joint tissue and maintaining articular structural integrity after being administered orally for 6 weeks.

Treatment started a week before MIA injection. Animals were randomized into five groups of 10 rats per group as G1=normal, G2=vehicle (0.5% CMC-Na solution), G3=Diclofenac (10 mg/kg, Lot #W08B043, Ward Hill, Mass.), G4=AMK (100 mg/kg) and G5=AMK (300 mg/kg), were orally gavaged with respective treatment. On the induction day, isoflurane (Lot #B66H15A, Piramal Enterprise Ltd. Andhra Pradesh, India) anesthetized rats were injected with 0.8 mg of MIA (Lot #A0352046, Acros Organics, New Jersey, USA) in 50 μL saline solution into the intra-articular pocket of left femorotibial (knee) joint using 26 G needle an hour after treatment. Normal control rats were injected with an equal volume of saline. Paw withdrawal thresholds as a result of constant pressure applied to the affected joint as a measure of pain sensitivity were taken once a week using Randall-Salitto Anesthesiometer (IITC, USA) and treatment lasted for 6 weeks. Body weights were measured once a week to calculate the respective weekly dosage of each group. Urine was collected at the end of study using metabolic cages. Blood samples were collected to isolate serum for biomarker analysis. At necropsy, animals were asphyxiated with CO₂ and the femorotibial joint was carefully dissected out, fixed in 10% buffered formalin and sent to Nationwide Histology (Veradale, Wash., USA) for further histopathology analysis. The fixed specimens were then decalcified with Calci-Clear Rapid for 1 and a half days and embedded in paraffin. Standardized 5 m serial sections were obtained at the medial and lateral mid-condylar level in the sagittal plane and were stained with hematoxylin and eosin (HE) and Safranin O-fast green to enable evaluation of proteoglycan content. A modified Mankin system (Mankin et al., 1981) was used to score structural and cellular alterations of articular components as indications of disease progression and/or treatment efficacy. The histological analysis was conducted by a certified Pathologist at Nationwide Histology.

### Example 60. Anti-Pain Sensitivity Activity of Composition AMK in MIA-Induced OA Model

Pain, one of the main cardinal symptoms of OA, was evidenced a week following model induction. As seen in Table 46, rats with an intra-articular injection of MIA without treatment showed a progressive increase in pain sensitivity as exhibited by the mean pain sensitivity values. Compared to the vehicle-treated normal control animals, rats with intra-articular 0.8 mg/joint MIA showed 34.6, 37.3, 41.4, 41.9 and 42.7% increases in pain sensitivity from week-1 to week-5, respectively. In contrast, all treatment groups showed statistically significant inhibition in pain sensitivity for all the weeks (Table 46). The highest inhibition in pain sensitivity was observed for rats treated with 300 mg/kg of the composition AMK. These reductions were compared against the vehicle-treated group and found to be 21.8%, 28.3%, 35.0%, 39.4% and 43.9% from week 1 to week-5, respectively, for rats treated with AMK composition at oral doses of 300 mg/kg/day. Rats administered with AMK at 100 mg/kg experienced 14.1%, 15.9%, 22.1%, 24.0% and 23.5% reductions in pain sensitivity from week 1 to week-5, respectively, when compared to vehicle-treated MIA rats. The observed pain relief was statistically significant at each data point examined for both the dosages. Diclofenac, the positive control, showed 19.7%, 24.1%, 28.3%, 30.8% and 31.1% reduction in pain sensitivity from week 1 to week-5, respectively, when compared to vehicle-treated MIA rats.

TABLE 46
Compression threshold for MIA-injected rats treated with AMK composition

| | | Knee compression sensitivity | | | | | |
|---|---|---|---|---|---|---|---|
| | Dose | (Mean ± SD) | | | | | |
| Group | (mg/kg) | Wk-0 | Wk-1 | Wk-2 | Wk-3 | Wk-4 | wk-5 |
| Normal | 0 | 243.9 ± 2.2 | 243.0 ± 1.7* | 244.8 ± 2.3* | 244.2 ± 3.4* | 241.3 ± 4.7* | 242.1 ± 4.2* |
| Control | | (0.05) | (52.4) | (59.4) | (70.5) | (72.3) | (74.4) |
| MIA | 0 | 243.8 ± 2.9 | 159.5 ± 2.4 | 153.4 ± 1.5 | 143.2 ± 0.9 | 140.1 ± 2.4 | 138.8 ± 1.4 |
| Diclofenac | 10 | 243.1 ± 2.4 | 190.9 ± 2.3* | 190.5 ± 2.6* | 183.8 ± 2.3* | 183.3 ± 1.7* | 182.0 ± 1.1* |
| | | (0.3) | (19.7) | (24.1) | (28.3) | (30.8) | (31.1) |
| AMK | 100 | 241.2 ± 1.1 | 181.9 ± 1.5* | 177.9 ± 2.9* | 174.9 ± 1.5* | 173.7 ± 2.4* | 171.4 ± 3.1* |
| | | (1.1) | (14.1) | (15.9) | (22.1) | (24.0) | (23.5) |
| AMK | 300 | 242.3 ± 1.4 | 194.3 ± 1.3* | 196.8 ± 3.5* | 193.3 ± 1.3* | 195.2 ± 1.7* | 199.8 ± 3.7* |
| | | (0.6) | (21.8) | (28.3) | (35.0) | (39.4) | (43.9) |

*P ≤ 0.00001. Values in the parenthesis are percent inhibition against vehicle treated MIA rats

### Example 61. Cartilage Protection and a Reduction in Proinflammatory Cytokine Activity by Composition AMK in MIA-Induced Rat Arthritis Model

ELISA assays for the detection of urinary CTX-II and IL-6 were described in the above examples. In this MIA-induced rat arthritis model, rats treated with AMK at 300 mg/kg orally resulted in statistically significant reductions in the cartilage degradation biomarker urinary CTX-II and the catabolic cytokine IL-6. When compared to vehicle-treated MIA rats, these reductions were found to be 31.9% and 22.5% for the AMK-treated rats at 300 mg/kg, respectively. At the lower dose of AMK (i.e. 100 mg/kg), there was a statistically significant reduction in serum IL-6. The Diclofenac-treated group (the positive control used for this model), followed a similar pattern to the lower dose of AMK (i.e. significant reduction in IL-6 with no impact on the urinary CTX-II).

TABLE 47
Changes in cytokine and cartilage degradation marker
after AMK treatment in MIA model

| | Dose | CTX-II | IL-6 |
|---|---|---|---|
| Group | (mg/kg) | (ng/g protein) | (pg/mL) |
| Normal | 0 | 282.3 ± 39.8 | 121.6 ± 9.6 |
| Control | | | |
| MIA+ | 0 | 240.8 ± 31.0 | 124.1 ± 8.4 |
| Diclofenac | 10 | 221.0 ± 29.5 | 114.2 ± 5.5* |
| AMK | 100 | 258.1 ± 45.6 | 114.6 ± 6.0* |
| AMK | 300 | 164.0 ± 17.6* | 98.3 ± 11.0** |

P ≤ 0.05 vs Vehicle;
** P ≤ 0.001 vs Vehicle

### Example 62. Improved Histological Findings as a Result of Composition Alpinia:Magnolia:Kochia (AMK) in MIA-Induced OA Model

Complementing the pain sensitivity reduction data, statistically significant improvements in articular cartilage matrix integrity were shown as reflected by the modified total Mankin score for animals treated with composition AMK at both the dosages. Structural abnormalities and fibrovascular proliferation were also significantly reduced in AMK groups. When the overall structural abnormalities (cartilage thickening or thinning, surface irregularity, fissure loss, degeneration, ulcerative necrosis, sever disorganization and chaotic appearance) were assessed, reductions of 41.1%, 33.1%, and 87.0% were observed for rats treated with Diclofenac (10 mg/kg), AMK (100 mg/kg) and AMK (300 mg/kg), respectively, (Table 48). The highest inhibition (72.5%) in catabolic inflammation and infiltration of inflammatory cells was observed for rats treated with AMK at 300 mg/kg as compared to the 42.8% from the Diclofenac group.

The extent of osteoclast activities and subchondral bone damage were minimal in MIA rats treated with AMK and the positive control drug. In contrast, various degrees of histopathological changes, including cellular degeneration and disorganization of the articular cartilage chondrocytes, depletion and collapse of the intracellular matrix, articular surface irregularities, osteophyte remodeling, and fibrillation of the subchondral bone, were observed for MIA-injected rats treated with vehicle. These changes in MIA rats were similar to the most common findings in human OA biology (Loeser et al., 2013). In Safranin −O staining, articular cartilage of AMK-treatment groups revealed minimum loss of staining intensity, indicating its ability to spare cartilage degradation. For instance, reductions of 34.6%, 31.0%, and 70.7% were observed in matrix GAG loss for Diclofenac (10 mg/kg), AMK (100 mg/kg), and AMK (300 mg/kg), respectively. Rats in the normal control groups treated with vehicle without MIA showed negligible changes in all the parameters examined. Closer to normal structure of the articular cartilage, subchondral bone of both tibia plateaus and femoral bone, and the surrounding joint structure appeared intact in this group of rats.

TABLE 48
Modified Mankin scores from histopathological findings for
MIA-induced rats treated with Alpinia:Magnolia:Kochia (AMK)

| | | Cartilage | | | |
|---|---|---|---|---|---|
| | Dose | destruction | GAG Loss | Bone Erosion | Inflammation |
| Group | (mg/kg) | (Mean ± SE) | (Mean ± SE) | (Mean ± SE) | (Mean ± SE) |
| Normal | 0 | 0.83 ± 0.45*** | 0.50 ± 0.26*** | 0.50 ± 0.16*** | 0.33 ± 0.13*** |
| Control | | (87.8) | (89.4) | (90.4) | (85.6) |
| MIA+ | 0 | 6.79 ± 0.26 | 4.71 ± 0.07 | 5.20 ± 0.28 | 2.29 ± 0.11 |
| Diclofenac | 10 | 4.00 ± 0.76* | 3.08 ± 0.57* | 3.08 ± 0.50* | 1.31 ± 0.22* |
| | | (41.10 | (34.6) | (40.8) | (42.8) |
| AMK | 100 | 4.54 ± 0.42** | 3.25 ± 0.32** | 3.42 ± 0.38* | 1.90 ± 0.35 |
| | | (33.1) | (31.0) | (34.2) | (17.0) |
| AMK | 300 | 0.88 ± 0.35*** | 1.38 ± 0.25*** | 0.54 ± 0.29*** | 0.63 ± 0.17*** |
| | | (87.0) | (70.7) | (89.6) | (72.5) |

*P ≤ 0.05;
**P ≤ 0.001;
† P ≤ 0.00001

### Example 63. Significance of Outcome from the MIA Model for Composition Alpinia:Magnolia:Kochia (AMK) and its Implication in OA

It is believed that at various stages of OA, all the three major structures of the joint (cartilage, subchondral bone and synovium) could be involved in the pathophysiology of the disease, which complicates the identification of a single biomarker that is indicative of a need for immediate therapeutic intervention at the early stage of the disease. Nevertheless, among all the major joint biomarkers observed, C-terminal telopeptide of type II collagen (CTX-II) has been by far the most studied and frequently referred to biomarker of cartilage degradation, and could be used for the purposes of diagnosis, determining the severity of disease or extent of disease progression, prognosis and monitoring the efficacy of treatment. CTX-II is primarily generated by matrix metalloproteinase activity during cartilage degradation in OA. It is known to show a close link with the catabolic/anabolic homeostasis and progression of articular cartilage degradation in OA patients. The direct correlation of CTX-II levels increased in serum, urine or synovial fluid level and articular cartilage degradation were reported both in pre-clinical and clinical studies (Oestergaard et al., 2006; Garnero et al., 2001), suggesting that plant extracts with inherent characteristics of reducing uCTX-II levels changed the phenotype of arthritis toward reduced catabolic degradation and increased anabolic regeneration and rebuilding by regulating homeostasis of chondrocytes, extracellular matrix, and articular cartilage.

Coupled with symptoms and biomarkers, histopathological analyses of articular cartilage, synovial membrane, and subchondral bone have been used to evaluate OA disease progression or to measure outcome of therapeutic interventions (Goldring et al., 2000). In the current disclosure, significant improvements to maintenance of the articular structural integrity of rats treated with the example, but not limited to, AMK composition were observed. These effects were demonstrated in the histopathology data as exhibited by limited loss, degeneration, or necrosis of chondrocytes, smoother articular cartilage surface, deeper and uniform stain of intracellular matrix, and close to normal contour of the subchondral bone. For obvious reasons, this minimal cartilage degradation was also supported by the significant reductions in pain sensitivity whereby the AMK composition achieved maximum pain relief. Furthermore, as demonstrated by the urine CTX-II biomarker data, a statistically significant reduction in the level of uCTX-II was also observed for rats treated with the composition AMK. Substantiating this statement in human clinical studies, urine CTX-II levels were well aligned with cartilage degradation and associated pain in OA patients. For example, urinary CTX-II concentrations were found elevated and associated with knee pain and function in subjects who underwent anterior cruciate ligament reconstruction. In these patients, decreased uCTX-II concentrations were correlated with decreased knee pain and improved function, providing meaningful prognosis (Chmielewski et al., 2012). Similarly, in a cross-sectional evaluation of biochemical markers of bone, cartilage, and synovial tissue metabolism in patients with knee osteoarthritis, uCTX-II was found significantly increased corresponding to disease severity and was correlated with changes in joint space narrowing (Garnero et al., 2001).

Considering the multifactorial nature of OA, it has previously been suggested that the ability to slow the progression of articular cartilage degeneration is more likely with a combination therapy than with any single component alone (Lippiello et al., 2000). The composition of bioactive standardized extracts from special blending ratios of Alpinia galanga, Magnolia officinalis, Piper nigrum, and Kochia scoparia may suit very well in this application. In fact, when the merit of formulating these two or three plant extracts was tested in the carrageenan-induced rat paw edema model, unexpected synergy in alleviating pain sensitivity was observed from the combination of these two or three plant extracts, exceeding the predicted result based on simply summing the effects observed for each of its constituents. Clinical and pre-clinical literature searches failed to predict the current disclosure of these plant extracts blended together as the compositions described in this patent. This signifies the novelty of the composition in maintaining articular structural integrity as reflected by the reduced uCTX-II levels accompanied by minimal pain sensitivity here again in this MIA-induced arthritis model. We believe that these medicinal plants may have complementary effects in regulating homeostasis of chondrocytes, extracellular matrix, articular cartilage, and phenotype of arthritis that lead to the prevention of articular cartilage degradation and the mitigation of associated symptoms, which could be translated to improved joint integrity, mobility and function.

### Example 64. Anti-Pain Activity of Topically Applied Plant Extracts in a Hot Plate Test

Repeated application of anti-inflammatory compounds or extracts topically at the site of thermal contact (noxious stimulus) may cause desensitization of the peripheral afferent pain receptors to produce a delay in response time. A longer change in reaction time could be interpreted as an anti-nociceptive effect of the applied compound. To evaluate whether prepared plant extracts and compositions could provide anti-nociceptive activity, rats received orally-active anti-inflammatory extracts formulated in 2% Aloe vera gel at 5% concentration topically on their hind paws. The applied preparations on each paw were massaged at least 60 times in a circular motion into the skin until the applied content appeared visually absorbed. The procedure was repeated 3 times, every 30 minutes, before placing the animals on a preheated hot plate set to 53° C. The paw withdrawal latency was calculated as the time elapsed from the initial placement of the rat onto the hotplate to the withdrawal (or licking or shaking) of the hind paw in response to the thermal stimulus. Animals were immediately removed when this response was observed. Those animals that did not display a response within 30 seconds were removed from the heated plate to prevent any tissue damage.

The anti-pain activity of medicinal plant extracts was tested on a hot plate set at 53° C. Test materials at 5% concentrations were topically applied at 20 μl/paw to the hind paws of both right and left paws of Sprague Dawley rats (n=10 per group). Applications of these extracts were carried out every 30 minutes for a total of 90 minutes. Within these 90 minutes, rats received a total of 60 μl/paw of the test article per rat. At 5% concentration, each rat received 3 mg/paw of the test articles. Due to the solubility characteristics of the compounds, two types of vehicles, V1=Dimethyl sulfoxide (DMSO)+propylene glycol (PG)+Aloe (2%), and V2=DMSO+OIL+PG, were used. Percent changes and P-values for statistical significance were determined using respective vehicles for each test material. The vehicle used for each material has been indicated in the parenthesis next to the test material. As depicted in Table 49, below, rats topically given pure Piperine showed 32.6% increase in paw withdrawal latency as compared to vehicle. This increase in anti-pain activity was similar to what was observed for the 5% ibuprofen (i.e. 22.4% increase in paw latency with 0.018 P-value). These increases in anti-pain activity were statistically significant. Alpinia galanga from ethanol extract and supercritical fluid extract-treated animals showed 17.1% and 32.8% increase in paw withdrawal latency with P-values 0.063 and 0.02, respectively. The Capsaicin-treated rats experienced 36.7% decrease in paw withdrawal latency. This percent change was statistically significant when compared to its respective vehicle. Those rats that received topical preparations of Magnolia officinalis showed 13.6% increase in sensitivity as compared to the respective vehicle controls.

These results indicate that Ibuprofen (positive control) and Alpinia galanga extract exhibited significant anti-nociceptive activities as evidenced by increased paw withdrawal latency in the hot plate test. Capsaicin (negative control) significantly decreased the paw withdrawal latency, as expected. Alpinia galanga extract could be utilized for topical pain relief for various indications.

TABLE 49
Paw withdrawal threshold as a measure of anti-pain
activity of medicinal plants in a hot plate test

| | | | | % | |
|---|---|---|---|---|---|
| Group | Dose (%) | N | Mean ± SD | Change ^{c} | P-values |
| Vehilce-1 (V1) | DMSO+ PG + | 10 | 7.13 ± 1.25 | — | — |
| | Aloe (2%) | | | | |
| Vehicle-2 (V2)-R1 | DMSO + OIL^{b} + PG | 10 | 6.01 ± 0.74 | — | — |
| Vehicle-2 (V2)-R2 | DMSO + OIL^{b} + PG | 10 | 4.88 ± 0.96 | — | — |
| Ibuprofen (V1) | 5% | 10 | 8.73 ± 1.37 | −22.4 | 0.018 |
| Ibuprofen (V2)-R2 | 5% | 10 | 6.05 ± 1.98 | −23.98 | 0.03 |
| Capsaicin (V1) | 0.5 | 10 | 4.51 ± 1.12 | 36.7 | 0.0002 |
| Diclofenac (V1) | 1 | 10 | 6.93 ± 1.50 | 2.8 | 0.763 |
| Menthol (V1) | 5 | 10 | 6.05 ± 1.70 | 15.1 | 0.144 |
| Ben-Gay | OTC^{a} | 10 | 7.00 ± 1.93 | 1.8 | 0.868 |
| Magnolia officinalis (V1) | 5 | 10 | 6.16 ± 1.37 | 13.6 | 0.135 |
| Alpinia galanga (V1) | 5 | 10 | 8.35 ± 1.36 | −17.1 | 0.063 |
| Alpinia galanga | 5 | 10 | 6.48 ± | −32.79 | 0.02 |
| (CO₂ ext) (V2)-R2 | | | | | |
| Piperine (V2)-R1 | 5 | 8 | 7.97 ± 1.86 | −32.6 | 0.011 |

^{a}OTC = over-the-counter product that contains Camphor, menthol and methyl salicylate at 5%, 10%, and 30% concentrations, respectively.
^{b}OIL = Medium chain triglyceride (MCT) oil derived from coconut oil
^{c} While the negative percent changes in the table are indications of increased paw withdrawal latency and hence increase pain relief, the positive percent changes reflect increased sensitivity.
R1/R2 = run 1/run 2

### Example 65. A 7-Day Repeated Oral Acute Maximum Tolerable Dose (MTD) Study of AP

Purpose-bred male and female CD-1 mice were purchased from Charles River at 8 weeks of age and used for the Maximum Tolerable Dose study. Following acclimation, mice were randomly assigned based on their body weight to the following respective groups: G1=Vehicle control (0.5% CMC), G2=Alpinia+pepper at 500 mg/kg and G3=750 mg/kg. Ten mice were placed in each group for this study. The test compound was suspended in 0.5% CMC and administered to mice at a volume of 350 μl/mouse. The vehicle group received 0.5% CMC. At baseline, the average body weights were 36.1±2.5 and 28.2±2.1 grams, for male and female mice, respectively. Body weights were monitored for a total of 4 measurements (i.e. baseline, 2, 3- and 7-days post challenge) after gavaging. Each group of mice were monitored for their physical activity and behavior after gavaging every day in both studies.

TABLE 50
Body weight measurements of mice treated with AP for 7-days

| | Dose | | Male (Mean ± SD) | | | Female (Mean ± SD) | | |
|---|---|---|---|---|---|---|---|---|
| Group | (mg/kg) | N | BL | 3dpc | 7dpc | BL | 3dpc | 7dpc |
| Vehicle | 0 | 10 | 36.5 ± 2.5 | 37.2 ± 2.8 | 37.6 ± 2.6 | 28.1 ± 2.1 | 29.1 ± 1.9 | 28.8 ± 1.6 |
| AP | 500 | 10 | 35.7 ± 2.8 | 35.3 ± 3.9 | 36.8 ± 3.8 | 28.2 ± 2.3 | 27.5 ± 2.1 | 26.9 ± 2.4 |
| AP | 750 | 10 | 36.3 ± 2.2 | 37.2 ± 2.6 | 35.7 ± 3.7 | 28.2 ± 1.7 | 28.3 ± 2.0 | 26.7 ± 2.2 |

Mice received these dosages for 7 consecutive days. No death was observed for the females in either of the AP dosage groups. However, 3 deaths occurred in male mice at the 750 mg/kg AP group on 2, 3- and 7-days post challenge. Gastric irritation and hemorrhage were observed in the deceased animals at Necropsy. At the end of the study, there were no significant body weight changes for the surviving males in the 750 mg/kg and 500 mg/kg AP groups (i.e. Vehicle BL=36.5±2.5 vs 7 dpc=37.6±2.6; 500 mg/kg AP BL=35.5±2.8 vs 7 dpc=36.8±3.8; 750 mg/kg AP BL=36.3±2.2 vs 35.7±3.7). In contrast, for the females, after 7-days of daily oral AP treatment, the percent body weight changes from the baseline were found decreased by 5.18 and 6.07% for the 500 and 750 mg/kg AP, respectively (i.e. i.e. Vehicle BL=28.1±2.1 vs 7 dpc=28.8±1.6; 500 mg/kg AP BL=28.2±2.3 vs 7 dpc=26.9±2.4; 750 mg/kg AP BL=28.2±1.7 vs 7 dpc=26.7±2.2). These body weight changes were statistically significant when compared to the vehicle group.

TABLE 51
Percent body weight changes of mice in a 7-day repeated daily oral MTD study

| | | N | | Study | % Bodyweight change at end of study* | | | |
|---|---|---|---|---|---|---|---|---|
| | Dose | Sex | | duration | | p- | | |
| Group | (mg/kg) | Male | Female | 7-days | Male | values | Female | p-values |
| Vehicle | 0 | 10 | 10 | 7-days | 2.83 | — | 2.30 | — |
| | 500 | 10 | 10 | 7-days | 3.09 | 0.91 | −5.18 | 0.02 |
| AP | 750 | 10 | 10 | 7-days | −0.57 | 0.28 | −6.07 | 0.01 |

*Negative numbers indicate percent reduction in body weight from baseline.
P-values were compared to vehicle

The surviving mice physically appeared normal after each gavage for both genders. The mice continued normal exploratory activity and behavior. These normal behaviors were continued for the remaining doses for both genders. These mice showed no changes in behavior or activity for the whole duration of treatment. At Necropsy, once the abdominal cavity was opened, the organs were subjected to gross examination for the surviving animals. No macroscopic (grossly visible) deviations from normal were observed. The appearance and Necropsy findings were comparable to the vehicle group.

According to the global pharmaceutical initiatives for MTD (Chapman et al., 2013), a 10% body weight loss at the end of a 7-day daily oral treatment from baseline would be considered a warning sign of toxicity. At the end of the current study, male and female CD-1 mice treated with oral doses of 500 mg/kg and 750 mg/kg AP showed less than 10% changes in their body weight from the baseline, while there were 3 animal deaths in the 750 mg/kg groups. As a result, we believe the MTD of AP composition is between 500-750 mg/kg.

### Example 66. A 7-Day Repeated Oral Acute Maximum Tolerable Dose (MTD) Study of AMK

Purpose-bred male and female CD-1 mice were purchased from Charles River at 8 weeks of age and used for the Maximum Tolerable Dose study. Following acclimation, mice were randomly assigned based on their body weight to two experiments. The first experiment included the following groups: G1=Vehicle control (5% DMSO+0.5% CMC), and G2=Alpinia:Magnolia:Kochia (AMK) at 2000 mg/kg. Eight mice were placed in each group for this study. The test compound was suspended in 5% DMSO+0.5% CMC and administered to mice at a volume of 350 μl/mouse. At baseline, the average body weights were 36.7±3.5 and 30.4±2.5 grams, for male and female mice, respectively. Body weights were monitored for a total of 3 measurements (i.e. baseline, 3 days post challenge and 7 days post challenge) after gavaging. Each group of mice was monitored for their physical activity and behavior after gavaging every day in both studies.

Both male and female mice were observed daily for 7 days for their physical appearances and behavior in both studies. Daily examination of mice for their physical condition and wellbeing showed no signs suggestive of toxicity or abnormality throughout the study period. Mice physically appeared normal after each gavage for both genders. The mice continued normal exploratory activity and behavior. These normal behaviors were continued for the remaining doses for both genders. The mice showed no changes in behavior or activity for the whole duration of treatment.

TABLE 52
Body weight measurements of mice treated with AMK for 7-days

| | Dose | | Male (Mean ± SD) | | | Female (Mean ± SD) | | |
|---|---|---|---|---|---|---|---|---|
| Group | (mg/kg) | N | BL | 3dpc | 7dpc | BL | 3dpc | 7dpc |
| Vehicle | 0 | 8 | 37.0 ± 3.6 | 37.8 ± 3.6 | 37.7 ± 3.6 | 30.6 ± 3.0 | 30.1 ± 2.8 | 30.7 ± 3.0 |
| AMK | 2000 | 8 | 36.6 ± 4.2 | 36.8 ± 4.4 | 37.0 ± 4.5 | 30.4 ± 2.0 | 30.2 ± 1.3 | 30.7 ± 1.3 |

As seen above, a similar pattern of body weight gain was observed for both genders and treatment groups. The rate of body weight gain was similar for both treatment groups for both genders. There were no statistically significant differences in body weight gain for either group. All mice in each group continued to maintain body weight for the duration of the study. By the end of the 7th day, the difference in body weight measurements between the baseline and the 7th day was insignificant (i.e. Male BL: 36.58±4.2 vs day 7:36.96±4.5; Female BL: 30.37±2.0 vs day 7:30.66±1.3).

TABLE 53
Percent body weight changes of
mice in a 7-day repeated daily oral MTD study

| | | N | | Study | % Bodyweight change | |
|---|---|---|---|---|---|---|
| | Dose | Sex | | duration | at end of study | |
| Group | (mg/kg) | Male | Female | 7-days | Male | Female |
| Vehicle | 0 | 8 | 8 | 7-days | 1.96 | −0.03 |
| AMK | 2000 | 8 | 8 | 7-days | 1.11 | 0.88 |

No morbidity or mortality was observed for the AMK treated mice. At Necropsy, once the abdominal cavity was opened, the organs were subjected to gross examination. No macroscopic (grossly visible) deviations from the normal were observed. The appearance and Necropsy findings for this groups were comparable to the vehicle group.

According to the global pharmaceutical initiatives for MTD (Chapman et al., 2013), a 10% body weight loss at the end of a 7-day daily oral treatment from baseline would be considered as a warning sign of toxicity. At the end of the current study, male and female CD-1 mice in the AMK group showed a comparable and insignificant body weight change to that of the vehicle group. Therefore, considering the normal physical activity, behavior and Necropsy findings in conjunction with maintenance of body weight at the end of the 7th day, it can be concluded AMK administered orally at 2000 mg/kg was tolerated through the course of a 7-day treatment in CD-1 mice. Hence, MTD for AMK is considered greater than 2000 mg/kg.

### Example 67. Human Clinical Study of the Compositions from the Individual Extracts of Alpinia, Pepper, Magnolia and Kochia and/or at Various Combinations of 2 to 3 of Those Extracts with Examples, but not Limited to, Compositions Alpinia:Pepper (AP) and Alpinia:Magnolia:Kochia (AMK)

In a clinical trial such as “A Double-blind Randomized Placebo and Positive Comparator Controlled Trial”, the Efficacy and Safety of proprietary compositions at 10-2000 mgs per dose, 2-3 times per day in Osteoarthritic patients will be evaluated. The study will evaluate symptom relief in pain severity on a 0-10 numeric Visual Analogue Scale (VAS), changes in pain severity, stiffness and joint function on the WOMAC scale by a subjective questionnaire. Objective measures of symptom improvement will be evaluated at baseline and at the end of the study for the range of motion by BIODEX and the distance walked in six minutes, and safety evaluations are also included. Biomarker measurements will also be carried on from the serum, synovial fluid and joint tissues before and after the treatment. The duration of the treatment shall be 1-12 weeks or 6-24 months according to the objective of the clinical output.

Before screening, the subjects must read and sign the IRB-approved Informed Consent Form. The study population consists of male and female subjects older than 18 and younger than 75 years, and in general good health as determined by a medical history. Female subjects of childbearing potential must have a negative urine pregnancy test at baseline. The goal of the study is to enroll at least 40 subjects per arm for meaningful statistical power.

The trial will have defined Inclusion criteria as follows: Male/Female healthy adults at 18 to 75 years of age; meet pain entry criteria; a history of knee joint pain for greater than 6 months; medial or lateral tibiofemoral joint line tenderness; unilateral knee pain 6/10 or greater, on average, on the visual analog scale (VAS), that interferes with function most days per week; Kellgren grade II or III radiographic changes of osteoarthritis; and willing to discontinue use of all analgesic medications (including over-the-counter [OTC] analgesics) except those provided as the study treatment and rescue medication specifically for study purposes.

### Primary Objective and Safety Evaluations:

Change in Pain Severity on 0-10 cm VAS
Change in pain severity, joint stiffness, and joint function on WOMAC Subscale (0-100), Change in WOMAC Total Score of all subscales.
Biomarkers of uCTX-II for cartilage degradation/protection; anabolic biomarkers ACAN, Sox-9, PIIANP and TGFβ; catabolic cytokines such as IL-1, IL-6, TNF-α, and MMP-13 from the serum and also synovial fluid will be measured. The global gene expression and protein expression profiles of cells/tissues from synovial fluid, synovial membrane, and cartilage for changes of catabolic and anabolic markers will be measured.
The joint space narrowing and total joint space area for ultimate proof of disease-modifying effects will also be measured.
Patient global assessment of response to treatment, Physician global assessment of response to treatment Improvement.
Change in joint function as measured by active and passive range of motion, distance walked in the 6-minute walk test. QOL: generic health status measure, the SF-36 and specific health status measures, the WOMAC
Complete Blood Count, Chemistry Panel with liver function tests, PT/INR, HCG and AE assessments.

### Data Analysis

In this study 10-200 subjects per group, randomized equally to receive single or multiple doses of individual extracts of Alpinia, Pepper, Magnolia and Kochia and/or at various combinations of 2 to 3 of those extracts with examples, but not limited to Alpinia:Pepper (AP) and Alpinia:Magnolia:Kochia (AMK), a positive control (either NTHE or dietary supplement active), and/or Placebo. If the attrition rate is 30% from the per-protocol population over the course of the 12-week study, there should be approximately more analyzable subjects per group. A power analysis was carried out to determine the effect size (difference between products in mean 12-week changes of efficacy endpoints) that would provide an 80% chance of obtaining a significant result of p≤0.05 with total analyzable subjects per group.

The statistical design parameters for this study are:
Alpha Level: 0.05 (p≤0.05 considered statistically significant)
Power: 0.8 (an 80% chance of obtaining significant p value)
Primary Null Hypothesis: Mean treatment duration changes for any supplement will equal the same duration for positive control and/or Placebo
Alternate Hypothesis: Changes are not equal between products
Statistical Test: Analysis of Covariance (power calculations based on unpaired Student t test)
Sample Size: 120 enrolled subjects, 40 in each product group

TABLE 54
Study Procedures

| | Procedure | | | | | |
|---|---|---|---|---|---|---|
| | Visit 1 | Visit 2 | Visit 3 | Visit 4 | Visit 5 | Visit 6 |
| | Visit Name | | | | | |
| | | | | | | Day 90 |
| | Screening | Day 0 | Day 14 | Day 30 | Day 60 | Exit Visit |
| | Day | Day | Day | Day | Day | Day |
| | −14 | 0 | 14 ± 1 | 30 ± 1 | 60 ± 2 | 90 ± 2 |
| Informed Consent | X | | | | | |
| Inclusion/Exclusion | X | X | | | | |
| Continuance Criteria | | X | X | X | X | |
| Medical History | X | | | | | |
| Physical Exam | X | | | | | X |
| Demography | X | | | | | |
| Height | X | | | | | |
| Weight | X | X | X | X | X | X |
| Vital signs | X | X | X | X | X | X |
| Identify target joint | X | | | | | |
| Chemistry panel with LFT | X | | | X | X | X |
| CBC with differential, PT/INR | X | | | X | X | X |
| Collect blood samples for | | X | | X | X | X |
| Cytokines | | | | | | |
| β-HCG Pregnancy Test | X | | | | | X |
| WOMAC pain subscale (5 | X | | | | | |
| items) | | | | | | |
| Complete WOMAC 3 subscales | | X | | X | X | X |
| 100 mm VAS Scale Daily | | X | | X | X | X |
| Assessment | | | | | | |
| Maximum Distance (feet) | | X | | X | X | X |
| walked in 6 minutes. | | | | | | |
| Concomitant Medications | X | X | X | X | X | X |
| Adverse Events/ Intercurrent | | | X | X | X | X |
| Illness | | | | | | |
| Dispense rescue medication | | X | | | | |
| Return Rescue Medication | | | X | X | X | X |
| Dispense Test Product | | X | | X | X | |
| Return Test Product | | | X | X | X | X |

### REFERENCES

1. Alford J W, Cole B J. Cartilage restoration, part I: basic science, historical perspective, patient evaluation and treatment options. Am J Sports Med. 2005; 33:295-306
2. Al-Snafi, Ali Esmail, A review on pharmacological activities of Kochia scoparia—a review, Indo American Journal of Pharmaceutical Sciences (2018), 5(4), 2213-2221.
3. Ayral X, Pickering E H, Woodworth T G, Mackillop N, Dougados M. Synovitis: a potential predictive factor of structural progression of medial tibiofemoral knee osteoarthritis—results of a 1 year longitudinal arthroscopic study in 422 patients. Osteoarthritis Cartilage. 2005 May; 13(5):361-7.
4. Bang K. H., Kim Y. K., Min B. S., et al. Antifungal activity of magnolol and honokiol. Archives of Pharmacal Research. 2000; 23:46-49.
5. Billinghurst R C, Dahlberg L, Ionescu M, Reiner A, Bourne R, Rorabeck C, et al. Enhanced cleavage of type II collagen by collagenases in osteoarthritic articular cartilage. J Clin Invest 1997; 99:1534-45.
6. Bolon, B.; Campagnuolo, G.; Zhu, L.; Duryea, D.; Zack, D.; Feige, U. Interleukin-Ibeta and tumor necrosis factor-alpha produce distinct, time-dependent patterns of acute arthritis in the rat knee. Vet. Pathol. 2004, 41, 235-243.
7. Bopaiah C. P., Pradhan N. Antimicrobial activity of honokiol and magnolol isolated from Magnolia officinalis. Phytotherapy Research. 2001; 15:139-141.
8. Brand, D. D.; Kang, A. H.; Rosloniec, E. F. Immunopathogenesis of collagen arthritis. Springer Semin. Immunopathol. 2003, 25, 3-18.
9. Chandrasekhar, S.; Harvey, A. K.; Hrubey, P. S. Intra-articular administration of interleukin-1 causes prolonged suppression of cartilage proteoglycan synthesis in rats. Matrix 1992, 12, 1-10.
10. Chen, D.; Shen, J.; Zhao, W.; Wang, T.; Han, L.; Hamilton, J. L.; Im, H. J. Osteoarthritis: Toward a comprehensive understanding of pathological mechanism. Bone Res. 2017, 5, 16044.
11. Chen, Y. C.; Tien, Y. J.; Chen, C. H.; Beltran, F. N.; Amor, E. C.; Wang, R. J.; Wu, D. J.; Mettling, C.; Lin, Y. L.; Yang, W. C. Morus alba and active compound oxyresveratrol exert anti-inflammatory activity via inhibition of leukocyte migration involving MEK/ERK signaling. BMC Complement. Altern. Med. 2013, 13, 45.
12. Cheon, B. S.; Kim, Y. H.; Son, K. S.; Chang, H. W.; Kang, S. S.; Kim, H. P. Effects ofprenylated flavonoids and biflavonoids on lipopolysaccharide-induced nitric oxide production from the mouse macrophage cell line RAW 264.7. Planta Med. 2000, 66, 596-600.
13. Chmielewski T L, Trumble T N, Joseph A M, Shuster J, Indelicato P A, Moser M W, Cicuttini F M, Leeuwenburgh C. Urinary CTX-II concentrations are elevated and associated with knee pain and function in subjects with ACL reconstruction. Osteoarthritis Cartilage. 2012 November; 20(11):1294-301.
14. Cho Y G, Cho M L, Min S Y, Kim H Y. Type II collagen autoimmunity in a mouse model of human rheumatoid arthritis. Autoimmun Rev. 2007 November; 7(1):65-70
15. Choi, Jongwon; Lee, Kyung-Tae; Jung, Hyun-Ju; Park, Hee-Sun; Park, Hee-Juhn; Anti-rheumatoid arthritis effect of the Kochia scoparia fruits and activity comparison of momordin Ic, its prosapogenin and sapogenin, Archives of Pharmacal Research (2002), 25(3), 336-342.
16. Christiansen B A, Bhatti S, Goudarzi R, Emami S. Management of Osteoarthritis with Avocado/Soybean Unsaponifiables. Cartilage. 2015 January; 6(1):30-44
17. Chung, K. O.; Kim, B. Y.; Lee, M. H.; Kim, Y. R.; Chung, H. Y.; Park, J. H.; Moon, J. O. In vitro and in vivo anti-inflammatory effect of oxyresveratrol from Morus alba L. J. Pharm.

Pharmacol. 2003, 55, 1695-1700.
18. Da R R, Qin Y, Baeten D, Zhang Y. B cell clonal expansion and somatic hypermutation of Ig variable heavy chain genes in the synovial membrane of patients with osteoarthritis. J Immunol. 2007 Jan. 1; 178(1):557-65.
19. Emmanuel L. Kuyinu, Ganesh Narayanan, Lakshmi S. Nair, Cato T. Laurencin. Animal models of osteoarthritis: classification, update, and measurement of outcomes. J Orthop Surg Res. 2016; 11: 19.
20. Fosang, A. J.; Stanton, H.; Little, C. B.; Atley, L. M. Neoepitopes as biomarkers of cartilage catabolism. Inflamm. Res. 2003, 52, 277-282.
21. Galil, S. M.; El-Shafey, A. M.; Hagrass, H. A.; Fawzy, F.; Sammak, A. E. Baseline serum level of matrix metalloproteinase-3 as a biomarker of progressive joint damage in rheumatoid arthritis patients. Int. J. Rheum. Dis. 2016, 19, 377-384.
22. Garnero P, Rousseau J C, Delmas P D. Molecular basis and clinical use of biochemical markers of bone, cartilage, and synovium in joint diseases. Arthritis Rheum. 2000 May; 43(5):953-68.
23. Garnero, P.; Piperno, M.; Gineyts, E.; Christgau, S.; Delmas, P. D.; Vignon, E. Cross sectional evaluation of biochemical markers of bone, cartilage, and synovial tissue metabolism in patients with knee osteoarthritis: Relations with disease activity and joint damage. Ann.

Rheum. Dis. 2001, 60, 619-626.
24. Gepstein, A; Shapiro, S; Arbel, G, Lahat, N; Livne, E. Expression of Matrix Metalloproteinases in Articular Cartilage of Temporomandibular and Knee Joints of Mice During Growth, Maturation, and Aging. Arthritis Rheum. 2002 December; 46(12):3240-3250.
25. Ghosh S, Rangan L. Alpinia: the gold mine of future therapeutics. Biotech. 2013 June; 3(3):173-185. doi: 10.1007/s13205-012-0089-x. Epub 2012 Sep. 18.
26. Glansbeek H L, van Beuningen H M, Vitters E L, van der Kraan P M, van den Berg W B.

Stimulation of Articular Cartilage Repair in Established Arthritis by Local Administration of Transforming Growth Factor-beta into Murine Knee Joints. Lab Invest. 1998; 78:133-142
27. Goldring M B. The role of the chondrocyte in osteoarthritis. Arthritis Rheum. 2000 September; 43(9):1916-26.
28. Goldring, M. B.; Fukuo, K.; Birkhead, J. R.; Dudek, E.; Sandell, L. J. Transcriptional suppression by interleukin-1 and interferon-gamma of type II collagen gene expression in human chondrocytes. J. Cell Biochem. 1994, 54, 85-99.
29. Goudarzi R, Reid A, McDougall J J. Evaluation of the novel avocado/soybean unsaponifiable Arthrocen to alter joint pain and inflammation in a rat model of osteoarthritis. PLoS One. 2018 Feb. 28; 13(2):e0191906
30. Guerne, P. A.; Carson, D. A.; Lotz, M. IL-6 production by human articular chondrocytes.

Modulation of its synthesis by cytokines, growth factors, and hormones in vitro. J. Immunol. 1990, 144, 499-505.
31. Guzman R E, Evans M G, Bove S, Morenko B, Kilgore K. Mono-iodoacetate-induced histologic changes in subchondral bone and articular cartilage of rat femorotibial joints: an animal model of osteoarthritis. Toxicol Pathol. 2003 November-December; 31(6):619-24.
32. Hosek, J.; Bartos, M.; Chudik, S.; Dall'Acqua, S.; Innocenti, G.; Kartal, M.; Kokoska, L.; Kollir, P.; Kutil, Z.; Landa, P.; et al. Natural compound cudraflavone B shows promising anti-inflammatory properties in vitro. J. Nat. Prod. 2011, 74, 614-619.
33. In L L, Arshad N M, Ibrahim H, Azmi M N, Awang K, Nagoor N H. 1′-acetoxychavicol acetate inhibits growth of human oral carcinoma xenograft in mice and potentiates cisplatin effect via proinflammatory microenvironment alterations. BMC Complement Altern Med. 2012 Oct. 9; 12:179. doi: 10.1186/1472-6882-12-179.
34. Joosten, L. A.; Helsen, M. M.; Saxne, T.; van De Loo, F. A.; Heinegard, D.; van Den Berg, W. B. IL-1 alpha beta blockade prevents cartilage and bone destruction in murine type II collagen-induced arthritis, whereas TNF-alpha blockade only ameliorates joint inflammation. J. Immunol. 1999, 163, 5049-5055.
35. Jordan K M, Arden N K, Doherty M, et al. EULAR Recommendations 2003: an evidence based approach to the management of knee osteoarthritis: Report of a Task Force of the Standing Committee for International Clinical Studies Including Therapeutic Trials (ESCISIT). Ann Rheum Dis. 2003 December; 62(12):1145-55.
36. Kalaiselvi, P.; Rajashree, K.; Bharathi Priya, L.; Padma, V. V. Cytoprotective effect of epigallocatechin-3-gallate against deoxynivalenol-induced toxicity through anti-oxidative and anti-inflammatory mechanisms in HT-29 cells. Food Chem. Toxicol. 2013, 56, 110-118
37. Kapoor, M.; Martel-Pelletier, J.; Lajeunesse, D.; Pelletier, J. P.; Fahmi, H. Role of proinflammatory cytokines in the pathophysiology of osteoarthritis. Nat. Rev. Rheumatol. 2011, 7, 33-42.
38. Kobayashi, M.; Squires, G. R.; Mousa, A.; Tanzer, M.; Zukor, D. J.; Antoniou, J.; Feige, U.; Poole, A. R. Role of interleukin-1 and tumor necrosis factor alpha in matrix degradation of human osteoarthritic cartilage. Arthritis Rheum. 2005, 52, 128-135.
39. Lefebvre, V.; Peeters-Joris, C.; Vaes, G. Modulation by interleukin 1 and tumor necrosis factor alpha of production of collagenase, tissue inhibitor of metalloproteinases and collagen types in differentiated and dedifferentiated articular chondrocytes. Biochim. Biophys. Acta 1990, 1052, 366-378.
40. Lepetsos, P.; Papavassiliou, A. G. ROS/oxidative stress signaling in osteoarthritis. Biochim.

Biophys. Acta 2016, 1862, 576-591.
41. Lippiello L, Woodward J, Karpman R, Hammad T A. In vivo chondroprotection and metabolic synergy of glucosamine and chondroitin sulfate. Clin Orthop Relat Res. 2000 December; (381):229-40.
42. Loeser R F. Osteoarthritis year in review 2013: biology. Osteoarthritis Cartilage. 2013 October; 21(10):1436-42.
43. M. Nagashekhar, H. N. Shivaprasad, An Important Medicinal Plant: A review. Biomed, 1 (1), 63-68, 2006.
44. M. Nagashekhar, H. N. Shivaprasad, An Important Medicinal Plant: A review. Biomed, 1 (1), 63-68, 2006.
45. Ma, M. J.; Liu, H. C.; Qu, X. Q.; Wang, J. L. Matrix metalloproteinase-3 gene polymorphism and its mRNA expression in rheumatoid arthritis. Genet. Mol. Res. 2015, 14, 15652-15659.
46. Mankin, H. J.; Dorfman, H.; Lippiello, L.; Zarins, A. Biochemical and metabolic abnormalities in articular cartilage from osteo-arthritic human hips. II. Correlation of morphology with biochemical and metabolic data. J. Bone Jt. Surg. Am. 1971, 53, 523-537.
47. Mathy-Hartert, M.; Hogge, L.; Sanchez, C.; Deby-Dupont, G.; Crielaard, J. M.; Henrotin, Y. Interleukin-lbeta and interleukin-6 disturb the antioxidant enzyme system in bovine chondrocytes: A possible explanation for oxidative stress generation. Osteoarthr. Cartil. 2008, 16, 756-763.
48. Matsuda H, Morikawa T, Managi H, Yoshikawa M. Antiallergic principles from Alpinia galanga: structural requirements of phenylpropanoids for inhibition of degranulation and release of TNF-alpha and IL-4 in RBL-2H3 cells. Bioorg Med Chem Lett. 2003 Oct. 6; 13(19):3197-202.
49. Matsuda, Hideaki; Dai, Yue; Ido, Yasuko; Ko, Sungkwon; Yoshikawa, Masayuki; Kubo, Michinori, Studies on Kochiae Fructus III. Antinociceptive and antiinflammatory effects of 70% ethanol extract and its component, momordin Ic from dried fruits of Kochia scoparia L., Studies on Kochiae Fructus III. Antinociceptive and antiinflammatory effects of 70% ethanol extract and its component, momordin Ic from dried fruits of Kochia scoparia L. Biological and Pharmaceutical Bulletin (1997), 20(10), 1086-1091.
50. McAlindon T E, Bannuru R R, Sullivan M C, et al. OARSI guidelines for the non-surgical management of knee osteoarthritis. Osteoarthritis Cartilage. 2014 March; 22(3):363-88.
51. Miyoshi, M.; Liu, S. Collagen-Induced Arthritis Models. Methods Mol. Biol. 2018, 1868, 3-7.
52. Mongkol, Rachsawan; et al.; Phytotoxic and antiphytopathogenic compounds from Thai Alpinia galanga (L.) Willd. Rhizomes. Weed Biology and Management (2015), 15(2), 87-93.
53. Nelson F, Dahlberg L, Laverty S, Reiner A, Pidoux I, Ionescu M, et al. Evidence for altered synthesis of type II collagen in patients with osteoarthritis. J Clin Invest 1998; 102:2115-25.
54. Oestergaard, S.; Chouinard, L.; Doyle, N.; Karsdal, M. A.; Smith, S. Y.; Qvist, P.; Tankó, L. B. The utility of measuring C-terminal telopeptides of collagen type II (CTX-II) in serum and synovial fluid samples for estimation of articular cartilage status in experimental models of destructive joint diseases. Osteoarthr. Cartil. 2006, 14, 670-679.
55. Oestergaard, S.; Chouinard, L.; Doyle, N.; Smith, S. Y.; Tankó, L. B.; Qvist, P. Early elevation in circulating levels of C-telopeptides of type II collagen predicts structural damage in articular cartilage in the rodent model of collagen-induced arthritis. Arthritis Rheum. 2006, 54, 2886-2890.
56. Park J., Lee J., Jung E., et al. In vitro antibacterial and anti-inflammatory effects of honokiol and magnolol against Propionibacterium sp. European Journal of Pharmacology. 2004; 496:189-195.
57. R. Satish, R. Dhananjayan, Alpinia: the gold mine of future therapeutics. Biomedicine, 23 (1/2), 91-96, 2003.
58. Roemer F W, Felson D T, Yang T, et al. The association between meniscal damage of the posterior horns and localized posterior synovitis detected on T1-weighted contrast-enhanced MRI—the MOST study. Semin Arthritis Rheum. 2013 June; 42(6):573-81.
59. Roemer F W, Guermazi A, Felson D T, et al. Presence of MRI-detected joint effusion and synovitis increases the risk of cartilage loss in knees without osteoarthritis at 30-month follow-up: the MOST study. Ann Rheum Dis. 2011 October; 70(10):1804-9
60. Rose, B. J.; Kooyman, D. L. A Tale of Two Joints: The Role of Matrix Metalloproteases in Cartilage Biology. Dis. Markers 2016, 2016, 4895050.
61. Rosloniec, E. F.; Cremer, M.; Kang, A. H.; Myers, L. K.; Brand, D. D. Collagen-induced arthritis. Curr. Protoc. Immunol. 2001
62. Sakkas L I, Platsoucas C D. The role of T cells in the pathogenesis of osteoarthritis. Arthritis Rheum. 2007 February; 56(2):409-24.
63. Saklatvala, J. Tumour necrosis factor alpha stimulates resorption and inhibits synthesis of proteoglycan in cartilage. Nature 1986, 322, 547-549.
64. Sandy J D, Plaas A H. Age-related changes in the kinetics of release of proteoglycans from normal rabbit cartilage explants. J Orthop Res. 1986 4(3):263-72.
65. Shivkanya Jaju, Nitin Indurwade, Dinesh Sakarkar, Neeraj Fuloria, Mohamad Ali, Isolation of galangogalloside from rhizomes of Alpinia galangal. International Journal of Green Pharmacy, 3 (2), 144-147, 2009.
66. Siebuhr, A. S.; Wang, J.; Karsdal, M.; Bay-Jensen, A. C.; Jin, Y.; Zheng, Q. Matrix metalloproteinase-dependent turnover of cartilage, synovial membrane, and connective tissue is elevated in rats with collagen induced arthritis. J. Transl. Med. 2012, 10, 195.
67. Smolen J S, Aletaha D, Barton A et al. Rheumatoid arthritis. Nat Rev Dis Primers. 2018 Feb. 8; 4:18001.
68. Syu W.-J., Jr., Shen C.-C., Lu J.-J., Lee G.-H., Sun C.-M. Antimicrobial and cytotoxic activities of neolignans from Magnolia officinalis. Chemistry and Biodiversity. 2004; 1:530-537.
69. V. M. Kale; A. G. Namdeo; Anti-arthritic effect of 1′-acetoxychavicol acetate in complete freunds adjuvant-induced arthritis in rats. International Journal of Pharma and Bio Sciences 5(4):P9-P19
70. van Beuningen H M1, van der Kraan P M, Arntz O J, van den Berg W B. Transforming growth factor-beta 1 stimulates articular chondrocyte proteoglycan synthesis and induces osteophyte formation in the murine knee joint. Lab Invest. 1994 August; 71(2):279-90.
71. van de Loo, F. A.; Arntz, O. J.; Otterness, I. G.; van den Berg, W. B. Protection against cartilage proteoglycan synthesis inhibition by antiinterleukin 1 antibodies in experimental arthritis. J.

Rheumatol. 1992, 19, 348-356.
72. Verdier M P, Seite S, Guntzer K, Pujol J P, Boumediene K. Immunohistochemical analysis of transforming growth factor beta isoforms and their receptors in human cartilage from normal and osteoarthritic femoral heads. Rheumatol Int. 2003; 25:118-124.
73. Visser K, van der Heijde D. Optimal dosage and route of administration of methotrexate in rheumatoid arthritis: a systematic review of the literature. Ann Rheum Dis. 2009 July; 68(7):1094-9.
74. W. Pompimon, J. Jomduang, U. Prawat, S. Mankhetkorn, Anti-Phytopthora capsici Activities and Potential Use as Antifungal in Agriculture of Alpinia galanga Swartz, Curcuma longa Linn, Boesenbergia pandurata Schut and Chromolaena odorata: Bioactivities Guided Isolation of Active Ingredients American Journal of Agricultural and Biological Sciences, 4 (1), 83-91, 2009.
75. Wei, S. T.; Sun, Y. H.; Zong, S. H.; Xiang, Y. B. Serum Levels of IL-6 and TNF-α May Correlate with Activity and Severity of Rheumatoid Arthritis. Med. Sci. Monit. 2015, 21, 4030-4038.
76. Wenham C Y, Conaghan P G. New horizons in osteoarthritis. Age Ageing. 2013 May; 42(3):272-8.
77. Yamanaka, H. Matsuda, Y. Tanaka, M. Sendo, W. Nakajima, H. Taniguchi, A. Kamatani, N. Serum matrix metalloproteinase 3 as a predictor of the degree of joint destruction during the six months after measurement, in patients with early rheumatoid arthritis. Arthritis Rheum. 2000, 43, 852-858
78. Yang X, Eilerman R G. Pungent principal of Alpinia galangal (L.) swartz and its applications. J Agric Food Chem. 1999 April; 47(4):1657-62.
79. Yang, J. A.; Choi, J. H.; Rhee, S. J. Effects of green tea catechin on phospholipase A2 activity and antithrombus in streptozotocin diabetic rats. J. Nutr. Sci. Vitaminol. (Tokyo) 1999, 45, 337-346.
80. Zhang W, Doherty M, Arden N, et al. EULAR evidence based recommendations for the management of hip osteoarthritis: report of a task force of the EULAR Standing Committee for International Clinical Studies Including Therapeutics (ESCISIT). Ann Rheum Dis. 2005 May; 64(5):669-81.
81. Zhang W, Moskowitz R W, Nuki G, Abramson S, et al. OARSI recommendations for the management of hip and knee osteoarthritis, part I: critical appraisal of existing treatment guidelines and systematic review of current research evidence. Osteoarthritis Cartilage. 2007 September; 15(9):981-1000.
82. Zhang W, Moskowitz R W, Nuki G, Abramson S, et al. OARSI recommendations for the management of hip and knee osteoarthritis, Part II: OARSI evidence-based, expert consensus guidelines. Osteoarthritis Cartilage. 2008 February; 16(2):137-62.
83. Zhang W, Nuki G, Moskowitz R W, Abramson S, et al. OARSI recommendations for the management of hip and knee osteoarthritis: part III: Changes in evidence following systematic cumulative update of research published through January 2009. Osteoarthritis Cartilage. 2010 April; 18(4):476-99.
84. Zhang, D. D. Ling, S. Zhang, H. P. Shi, H. X. Xue, Y. L. Yang, X. L. Xu, J. W. Bian, K. Effects of total flavones from Morus alba L. On inflammation reaction of macrophages. Shizhen Guoyi Guoyao 2010, 21, 2787-2790.
85. Argoff C E. Topical analgesics in the management of acute and chronic pain. Mayo Clin Proc. 2013 February; 88(2):195-205.
86. Chapman K., Sewell F., Allais L, et al. A global pharmaceutical company initiative: an evidence-based approach to define the upper limit of body weight loss in short term toxicity studies. Regul Toxicol Pharmacol. 2013 October; 67(1):27-38.
87. Fox L T, Gerber M, du Preez J L, du Plessis J, Hamman J H. Skin permeation enhancement effects of the gel and whole-leaf materials of Aloe vera, Aloe marlothii and Aloe ferox. J Pharm Pharmacol. 2015 January; 67(1):96-106
88. Harirforoosh S, Asghar W, Jamali F. Adverse effects of nonsteroidal anti-inflammatory drugs: an update of gastrointestinal, cardiovascular and renal complications. J Pharm Sci. 2013; 16(5):821-47.
89. Rodriguez-Merchan E C. Topical therapies for knee osteoarthritis. Postgrad Med. 2018 September; 130(7):607-612.
90. Stanos S P, Galluzzi K E. Topical therapies in the management of chronic pain. Postgrad Med. 2013 July; 125(4 Suppl 1):25-33.

### SEQUENCE LISTING

The Sequence Listing found in File Name “SequenceListingTextASCII3”, created on Sep. 13, 2021 and which is 3.67 KB in size, is incorporated herein by reference.

1. A composition for joint health, comprising a combination of an Alpinia extract enriched for one or more phenylpropanoids; a Magnolia extract enriched for one or more bisphenolic lignans; and a Kochia extract enriched for one or more triterpenoid saponins.

2. The composition according to claim 1, wherein the Alpinia extract, or Magnolia extract or Kochia extract in the composition are in a range of 1%-98% by weight of each extract with the optimized weight ratio of Alpinia:Magnolia:Kochia (AMK) at 2:4:3 (22.2%:44.4%:33.3%) or 4:3:3 (40%:30%:30%) or 5:4:4 (38.4%:30.8%:30.8%).

3. The composition according to claim 1, wherein the Alpinia extract is from Alpinia galanga, the Magnolia extract is from Magnolia officinalis and Kochia extract is from Kochia scoparia.

4. The composition according to claim 1, wherein the Alpinia extract is comprised of 0.01% to 99.9% of phenylpropanoids.

5. The composition according to claim 1, wherein the Magnolia extract is comprised of 0.01% to 99.9% of bisphenolic lignans.

6. The composition according to claim 1, wherein the Kochia extract is comprised of 0.01% to 99.9% of triterpenoid saponins.

7. The composition according to claim 1, wherein the one or more phenylpropanoids from the Alpinia extract is 1′-acetoxychavicol acetate, or galangal acetate, or p-hydroxy cinnamaldehyde, or 3,5-dihydroxystilbene or any combination thereof.

8. The composition according to claim 1, wherein the one or more bisphenolic lignans from the Magnolia extract is magnolol or honokiol, or a combination thereof.

9. The composition according to claim 1, wherein the one or more triterpenoid saponins from the Kochia extract is Bassiasaponin A; or Bassiasaponin B; or Kochioside A; or Kochioside B; or Kochioside C; or Kochianoside I; or Scoparianos A; or Scoparianoside B; or Scoparianoside C; or Momordin Ic; or Kochianoside I; or Kochianoside II; or Kochianoside III; or Kochianoside IV; or 2′-O-glucopyranosyl momordin Ic; or 2′-O-Glucopyranosylmomordin IIc, or a combination thereof.

10. The composition claim 1, wherein the phenylpropanoids are enriched from a plant species selected from the group consisting of Alpinia galanga, Alpinia officinarum, Boesenbergia rotunda, Kaempferia galanga, Alpinia oxyphylla, Alpinia abundiflora, Alpinia acrostachya, Alpinia caerulea, Alpinia calcarata, Alpinia conchigera, Alpinia globosa, Alpinia javanica, Alpinia melanocarpa, Alpinia mutica, Alpinia nigra, Alpinia nutans, Alpinia petiolate, Alpinia purpurata, Alpinia pyramidata, Alpinia rafflesiana, Alpinia speciosa, Alpinia vittata, Alpinia zerumbet, Alpinia zingiberina, or a combination thereof.

11. The composition claim 1, wherein the bisphenolic lignans are enriched from a plant species selected from the group consisting of Magnolia officinalis, Magnolia acuminate, Magnolia biondii, Magnolia coco, Magnolia denudate, Magnolia fargesii, Magnolia garrettii, Magnolia grandiflora, Magnolia henryi, Magnolia liliflora, Magnolia kachirachirai, Magnolia Kobus, Magnolia obovata, Magnolia praecocissima, Magnolia pterocarpa, Magnolia pyramidata, Magnolia rostrate, Magnolia salicifolia, Magnolia sieboldii, Magnolia soulangeana, Magnolia stellate, Magnolia virginiana, prod. of degradation of birch lignin, Acanthus ebracteatus, Aptosimum spinescens, Aralia bipinnata, Araucaria angustifolia, Araucaria araucana, Artemisia absinthium, Haplophyllum acutifolium, Haplophyllum perforatum, Liriodendron tulipifera, Krameria cystisoides, Perilla frutescens, Lawsonia inermis Myristica fragrans (nutmeg), Parakmeria yunnanensis (preferred genus name Magnolia), Persea japonica, Piper futokadsura, Piper wightii, Rollinia mucosa, Sassafras randaiense, Scrophularia albida-colchica, Stellera chamaejasme, Syringa velutina, Syzygium cumini, Talauma gloriensis, Virola elongate, Urbanodendron verrucosum, Wikstroemia sikokiana or a combination thereof.

12. The composition claim 1, wherein the triterpenoid saponins are enriched from a plant species selected from the group consisting of Kochia scoparia, Bassia scoparia, Bassia angustifolia, Momordica cochinchinensis, Bassia dinteri, Bassia eriophora, Bassia hyssopifolia, Bassia indica, Bassia laniflora, Bassia lasiantha, Bassia littorea, Bassia muricata, Bassia odontoptera, Bassia pilosa, Bassia prostrata, Bassia salsoloides, Bassia stellaris, Bassia tianschanica, Bassia tomentosa, Bassia villosissima or a combination thereof.

13. The composition of claim 1, wherein the phenylpropanoids, bisphenolic lignans, and triterpenoid saponins. are enriched from a plant part selected from the group consisting of leaves, bark, trunk, trunk bark, stem, stem bark, twigs, tubers, root, rhizome, root bark, bark surface, young shoots, seed, fruit, androecium, gynoecium, calyx, stamen, petal, sepal, carpel (pistil), flower, or any combination thereof.

14. The composition according to claim 1, wherein the Alpinia extract, the Magnolia extract and the Kochia extract in the composition are extracted with any suitable solvent, including supercritical fluid of CO₂, water, methanol, ethanol, alcohol, a water-mixed solvent or a combination thereof.

15. The composition according to claim 1, wherein one or more phenylpropanoids; one or more bisphenolic lignans; and one or more triterpenoid saponins are enriched individually or in combination by solvent partition, precipitation, distillation, evaporation, column chromatograph with silica gel, XAD, HP20, LH20, C-18, alumina oxide, polyamide and CG161 resins.

16. The composition according to claim 1, wherein the composition further comprises a pharmaceutically or nutraceutically acceptable active, adjuvant, carrier, diluent, or excipient, wherein the pharmaceutical or nutraceutical formulation comprises from about 0.1 weight percent (wt %) to about 99.9 wt % of active compounds from the 3-extracts composition.

17. The composition of claim 16, wherein the active, adjuvant, excipient or carrier is selected from one or more of Cannabis sativa oil or CBD/THC, turmeric extract or curcumin, terminalia extract, willow bark extract, Devil's claw root extract, Cayenne Pepper extract or capsaicin, Prickly Ash bark extract, philodendra bark extract, hop extract, Boswellia extract, Morus alba extract, Acacia catechu extract, Scutellaria baicalensis extract, rose hips extract, rosemary extract, green tea extract, Sophora extract, Mentha or Peppermint extract, ginger or black ginger extract, green tea or grape seed polyphenols, bakuchiol or Psoralea seed extract, fish oil, glucosamine sulfate, glucosamine hydrochloride, N-acetylglucosamine, chondroitin chloride, chondroitin sulfate, methylsulfonyl methane (MSM), hyaluronic acid, undenatured or denatured collagen, Omega-3 or Omega-6 Fatty Acids, Krill oil, Egg Shell Membrane (ESM), gamma-linolenic acid, Perna Canaliculus (Green-Lipped Mussel), SAMe, avocado/soybean unsaponifiable (ASU) extract, citrus bioflavonoids, Acerola concentrate, astaxanthin, pycnogenol, vitamin C, vitamin D, vitamin E, vitamin K, vitamin B, vitamin A, L-lysine, calcium, manganese, Zinc, mineral amino acid chelate(s), amino acid(s), boron and boron glycinate, silica, probiotics, Camphor, Menthol, calcium-based salts, silica, histidine, copper gluconate, CMC, beta-cyclodextrin, cellulose, dextrose, saline, water, oil, shark and bovine cartilage.

18. The composition according to claim 1, wherein the composition is formulated as a tablet, hard capsule, soft gel capsule, powder, or granule, compressed tablet, pill, chewing gum, sashay, wafer, bar, or liquid form, tincture, aerial spread, semi solid, semi liquid, solution, emulsion, cream, lotion, ointment, gel base or like form.

19. The method according to claim 1, wherein the route of the administration is selected from the group consisting of oral, topical, suppository, intravenous, intradermic, intragastric, intramuscular, intraperitoneal, and intravenous.

20. The joint heath composition according to claim 1, comprising a method for treating, managing, promoting joint health in a mammal, comprising administering an effective amount of a composition from 0.01 mg/kg to 500 mg/kg body weight of the mammal.

21. The joint heath composition according to claim 1, comprising a method for maintaining catabolic/anabolic biomarker homeostasis by reducing or controlling catabolic biomarkers TNF-α, IL-1β, IL-6, aggrecanase and matrix metalloproteinase (MMP)—MMP13, MMP9, MMP3, MMP1, uCTX-II and ADAMTS4; and by increasing or enhancing or promoting anabolic biomarkers: SOX 9, TGF-β1, ACAN, COL2A1, and PIIANP of a mammal.

22. The joint heath composition according to claim 1, comprising a method for maintaining cartilage homeostasis, inducing cartilage synthesis (and hence, anabolic effect) and inhibiting the catabolic process of degradation and broken down, protecting extracellular matrix integrity, and joint cartilage, minimizing cartilage degradation, alleviating cartilage breakdown, and initiating or promoting or enhancing cartilage synthesis, cartilage renewal and cartilage rebuild, repairing damaged cartilage, maintaining, rebuilding and repairing extra cellular matrix of joint tissue, revitalizing joints structure, maintaining steady blood flow to joints, promoting health joints by protecting cartilage integrity, balancing anabolic and catabolic processes, maintaining synovial fluid for joint lubrication in a mammal, diminishing the action of enzymes and proinflammatory cytokines that affect joint health of a mammal.

23. The joint heath composition according to claim 1, comprising a method for improving joint movement or physical function, maintaining joint health and mobility into old age, supporting, protecting or promoting joint comfort, alleviating joint pain, reducing joint friction, alleviating joint stiffness, improving joint range of motion or flexibility, promoting mobility, reducing inflammation, reducing oxidative stress, reducing and protecting joint wear and tear, managing or treating osteoarthritis or rheumatoid arthritis, preventing osteoarthritis or rheumatoid arthritis, or reversing the progression of osteoarthritis or rheumatoid arthritis; Preventing and treating juvenile rheumatoid arthritis, Still's disease, psoriatic arthritis, reactive arthritis, septic arthritis, Reiter's syndrome, Behcet's syndrome, or Felty's syndrome or the like of a mammal.

24. A composition for joint health, comprising a combination of an Alpinia extract enriched for one or more phenylpropanoids; and a Piper extract enriched for one or more alkaloids.

25. The composition according to claim 24, wherein the Alpinia extract, and the Piper extract in the composition is 99:1 to 1:99 in weight ratios.

26. The composition according to claim 24, wherein the Alpinia extract is from Alpinia galanga, Piper extract is from Piper nigrum.

27. The composition according to claim 24 wherein the Alpinia extract is comprised of 0.01% to 99.9% of phenylpropanoids.

28. The composition according to claim 24 wherein the Piper extract is comprised of 0.01% to 99.9% of alkaloids.

29. The composition according to claim 24, wherein the one or more phenylpropanoids from the Alpinia extract is 1′-acetoxychavicol acetate, or galangal acetate, or p-hydroxy cinnamaldehyde, or 3,5-Dihydroxystilbene or any combination thereof.

30. The composition according to claim 24, wherein the one or more piperidine alkaloids from the Piper extract is Piperine, or Chavicine or isochavicine, or isopiperine, or coumaperine, or Feruperine, or Piperanine, or Piperettine, or Pipersintenamide, or Piperdardine, or Pipernonaline, or Pipertipine or a combination thereof.

31. The composition claim 24, wherein the phenylpropanoids are enriched from a plant species selected from the group consisting of Alpinia galanga, Alpinia officinarum, Boesenbergia rotunda, Kaempferia galanga, Alpinia oxyphylla, Alpinia abundiflora, Alpinia acrostachya, Alpinia caerulea, Alpinia calcarata, Alpinia conchigera, Alpinia globosa, Alpinia javanica, Alpinia melanocarpa, Alpinia mutica, Alpinia nigra, Alpinia nutans, Alpinia petiolate, Alpinia purpurata, Alpinia pyramidata, Alpinia rafflesiana, Alpinia speciosa, Alpinia vittata, Alpinia zerumbet, Alpinia zingiberina, or a combination thereof.

32. The composition claim 24, wherein the one or more piperidine alkaloids are enriched from Piper nigrum, Piper longum, Piper amalgo, Piper aurantiacum, Piper chaba, Piper capense, Piper crassinervium, Piper guineense, Piper methysticum, Piper novae-hollandiae, Piper peepuloides, Piper ponapense, Piper puberulum, Piper retrofractum, Piper sintenense, Piper tuberculatum, Piper hancei, Glycine max, Petrosimonia monandra, Mentha piperata, silocaulon absimile, and Ulocladium sp or a combination thereof.

33. The composition of claim 24, wherein the phenylpropanoids, and alkaloids are enriched from a plant part selected from the group consisting of leaves, bark, trunk, trunk bark, stem, stem bark, twigs, tubers, root, rhizome, root bark, bark surface, young shoots, seed, fruit, androecium, gynoecium, calyx, stamen, petal, sepal, carpel (pistil), flower, or any combination thereof.

34. The composition according to claim 24, wherein the Alpinia extract, the Piper extract in the composition are extracted individually or in combination with any suitable solvent, including supercritical fluid of CO₂, water, methanol, ethanol, alcohol, a water-mixed solvent or a combination thereof.

35. The composition according to claim 24, wherein one or more phenylpropanoids; one or more alkaloids are enriched individually or in combination by solvent partition, precipitation, distillation, evaporation, column chromatograph with silica gel, XAD, HP20, LH20, C-18, alumina oxide, polyamide, CG161 and ion exchange resins.

36. The composition according to claim 24, wherein the composition further comprises a pharmaceutically or nutraceutically acceptable active, adjuvant, carrier, diluent, or excipient, wherein the pharmaceutical or nutraceutical formulation comprises from about 0.1 weight percent (wt %) to about 99.9 wt % of active compounds from Alpinia and Piper extract composition.

37. The composition of claim 36, wherein the active, or adjuvant, or excipient or carrier is selected from one or more of Cannabis sativa oil or CBD/THC, turmeric extract or curcumin, terminalia extract, willow bark extract, Devil's claw root extract, Cayenne Pepper extract or capsaicin, Prickly Ash bark extract, philodendra bark extract, hop extract, Boswellia extract, Morus alba extract, Acacia catechu extract, Scutellaria baicalensis extract, rose hips extract, rosemary extract, green tea extract, Sophora extract, Mentha or Peppermint extract, ginger or black ginger extract, green tea or grape seed polyphenols, bakuchiol or Psoralea seed extract, fish oil, glucosamine sulfate, glucosamine hydrochloride, N-acetylglucosamine, chondroitin chloride, chondroitin sulfate, methylsulfonyl methane (MSM), hyaluronic acid, undenatured or denatured collagen, Omega-3 or Omega-6 Fatty Acids, Krill oil, Egg Shell Membrane (ESM), gamma-linolenic acid, Perna Canaliculus (Green-Lipped Mussel), SAMe, avocado/soybean unsaponifiable (ASU) extract, citrus bioflavonoids, Acerola concentrate, astaxanthin, pycnogenol, vitamin C, vitamin D, vitamin E, vitamin K, vitamin B, vitamin A, L-lysine, calcium, manganese, Zinc, mineral amino acid chelate(s), amino acid(s), boron and boron glycinate, silica, probiotics, Camphor, Menthol, calcium-based salts, silica, histidine, copper gluconate, CMC, beta-cyclodextrin, cellulose, dextrose, saline, water, oil, shark and bovine cartilage.

38. The composition according to claim 24, wherein the composition is formulated as a tablet, hard capsule, soft gel capsule, powder, granule, compressed tablet, pill, chewing gum, sashay, wafer, bar, or liquid form, tincture, aerial spread, semi solid, semi liquid, solution, emulsion, cream, lotion, ointment, gel base or like form.

39. The method according to claim 24, wherein the route of the administration is selected from the group consisting of oral, topical, suppository, intravenous, intradermic, intragastric, intramuscular, intraperitoneal, and intravenous.

40. The joint heath composition according to claim 24, comprising a method for treating, managing, promoting joint health in a mammal, comprising administering an effective amount of a composition from 0.01 mg/kg to 500 mg/kg body weight of the mammal.

41. The joint heath composition according to claim 24, comprising a method for maintaining catabolic/anabolic biomarker homeostasis by reducing or controlling catabolic biomarkers TNF-α, IL-1β, IL-6, aggrecanase and matrix metalloproteinase (MMP)—MMP13, MMP9, MMP3, MMP1, uCTX-II and ADAMTS4; and by enhancing and promoting anabolic biomarkers: SOX 9, TGF-β1, ACAN, COL2A1, and PIIANP of a mammal.

42. The joint heath composition according to claim 24, comprising a method for maintaining cartilage homeostasis, inducing cartilage synthesis (and hence, anabolic effect) and inhibiting the catabolic process of degradation and broken down, protecting extracellular matrix integrity, and joint cartilage, minimizing cartilage degradation, alleviating cartilage breakdown, and initiating or promoting or enhancing cartilage synthesis, cartilage renewal and cartilage rebuild, repairing damaged cartilage, maintaining, rebuilding and repairing extra cellular matrix of joint tissue, revitalizing joints structure, maintaining steady blood flow to joints, promoting health joints by protecting cartilage integrity, balancing anabolic and catabolic processes, maintaining synovial fluid for joint lubrication in a mammal, diminishing the action of enzymes and proinflammatory cytokines that affect joint health of a mammal.

43. The joint heath composition according to claim 24, comprising a method for improving joint movement or physical function, maintaining joint health and mobility into old age, supporting, protecting or promoting joint comfort, alleviating joint pain, reducing joint friction, alleviating joint stiffness, improving joint range of motion or flexibility, promoting mobility, reducing inflammation, reducing oxidative stress, reducing and protecting joint wear and tear, managing or treating osteoarthritis or rheumatoid arthritis, preventing osteoarthritis or rheumatoid arthritis, or reversing the progression of osteoarthritis or rheumatoid arthritis; Preventing and treating juvenile rheumatoid arthritis, Still's disease, psoriatic arthritis, reactive arthritis, septic arthritis, Reiter's syndrome, Behcet's syndrome, or Felty's syndrome or the like of a mammal.

44. A composition for joint health, comprising an Alpinia extract enriched for one or more phenylpropanoids.

45. The composition according to claim 44, wherein the Alpinia extract is comprised of 0.01% to 99.9% of phenylpropanoids.

46. The composition according to claim 44, wherein the one or more phenylpropanoids from the Alpinia extract is 1′-acetoxychavicol acetate, or galangal acetate, or p-hydroxy cinnamaldehyde, or 3,5-dihydroxystilbene or any combination thereof.

47. The composition claim 44, wherein the phenylpropanoids are enriched from a plant species selected from the group consisting of Alpinia galanga, Alpinia officinarum, Boesenbergia rotunda, Kaempferia galanga, Alpinia oxyphylla, Alpinia abundiflora, Alpinia acrostachya, Alpinia caerulea, Alpinia calcarata, Alpinia conchigera, Alpinia globosa, Alpinia javanica, Alpinia melanocarpa, Alpinia mutica, Alpinia nigra, Alpinia nutans, Alpinia petiolate, Alpinia purpurata, Alpinia pyramidata, Alpinia rafflesiana, Alpinia speciosa, Alpinia vittata, Alpinia zerumbet, Alpinia zingiberina, or a combination thereof.

48. The composition of claim 44, wherein the phenylpropanoids are enriched from a plant part selected from the group consisting of leaves, bark, trunk, trunk bark, stem, stem bark, twigs, tubers, root, rhizome, root bark, bark surface, young shoots, seed, fruit, androecium, gynoecium, calyx, stamen, petal, sepal, carpel (pistil), flower, or any combination thereof.

49. The composition according to claim 44, wherein the Alpinia extract in the composition are extracted with any suitable solvent, including supercritical fluid of CO₂, water, methanol, ethanol, alcohol, a water-mixed solvent or a combination thereof.

50. The composition according to claim 44, wherein one or more phenylpropanoids are enriched by solvent partition, precipitation, distillation, evaporation, column chromatograph with silica gel, XAD, HP20, LH20, C-18, alumina oxide, polyamide, CG161 and ion exchange resins.

51. The composition according to claim 44, wherein the composition further comprises a pharmaceutically or nutraceutically acceptable active, adjuvant, carrier, diluent, or excipient, wherein the pharmaceutical or nutraceutical formulation comprises from about 0.1 weight percent (wt %) to about 99.9 wt % of active compounds from the Alpinia extract composition.

52. The composition of claim 51, wherein the active, adjuvant, excipient or carrier is selected from one or more of Cannabis sativa oil or CBD/THC, turmeric extract or curcumin, terminalia extract, willow bark extract, Devil's claw root extract, Cayenne Pepper extract or capsaicin, Prickly Ash bark extract, philodendra bark extract, hop extract, Boswellia extract, Morus alba extract, Acacia catechu extract, Scutellaria baicalensis extract, rose hips extract, rosemary extract, green tea extract, Sophora extract, Mentha or Peppermint extract, ginger or black ginger extract, green tea or grape seed polyphenols, bakuchiol or Psoralea seed extract, fish oil, glucosamine sulfate, glucosamine hydrochloride, N-acetylglucosamine, chondroitin chloride, chondroitin sulfate, methylsulfonyl methane (MSM), hyaluronic acid, undenatured or denatured collagen, Omega-3 or Omega-6 Fatty Acids, Krill oil, Egg Shell Membrane (ESM), gamma-linolenic acid, Perna Canaliculus (Green-Lipped Mussel), SAMe, avocado/soybean unsaponifiable (ASU) extract, citrus bioflavonoids, Acerola concentrate, astaxanthin, pycnogenol, vitamin C, vitamin D, vitamin E, vitamin K, vitamin B, vitamin A, L-lysine, calcium, manganese, Zinc, mineral amino acid chelate(s), amino acid(s), boron and boron glycinate, silica, probiotics, Camphor, Menthol, calcium-based salts, silica, histidine, copper gluconate, CMC, beta-cyclodextrin, cellulose, dextrose, saline, water, oil, shark and bovine cartilage.

53. The composition according to claim 44, wherein the composition is formulated as a tablet, hard capsule, soft gel capsule, powder, granule, compressed tablet, pill, chewing gum, sashay, wafer, bar, or liquid form, tincture, aerial spread, semi solid, semi liquid, solution, emulsion, cream, lotion, ointment, gel base or like form.

54. The method according to claim 44, wherein the route of the administration is selected from the group consisting of oral, topical, suppository, intravenous, intradermic, intragastric, intramuscular, intraperitoneal, and intravenous.

55. The joint heath composition according to claim 44, comprising a method for treating, managing, promoting joint health in a mammal, comprising administering an effective amount of a composition from 0.01 mg/kg to 500 mg/kg body weight of the mammal.

56. The joint heath composition according to claim 44, comprising a method for maintaining catabolic/anabolic biomarker homeostasis by reducing or controlling catabolic biomarkers TNF-α, IL-1β, IL-6, aggrecanase and matrix metalloproteinase (MMP)—MMP13, MMP9, MMP3, MMP1, uCTX-II and ADAMTS4; and by enhancing and promoting anabolic biomarkers: SOX 9, TGF-β1, ACAN, COL2A1, and PIIANP of a mammal.

57. The joint heath composition according to claim 44, comprising a method for maintaining cartilage homeostasis, inducing cartilage synthesis (and hence, anabolic effect) and inhibiting the catabolic process of degradation and broken down, protecting extracellular matrix integrity, and joint cartilage, minimizing cartilage degradation, alleviating cartilage breakdown, and initiating or promoting or enhancing cartilage synthesis, cartilage renewal and cartilage rebuild, repairing damaged cartilage, maintaining, rebuilding and repairing extra cellular matrix of joint tissue, revitalizing joints structure, maintaining steady blood flow to joints, promoting health joints by protecting cartilage integrity, balancing anabolic and catabolic processes, maintaining synovial fluid for joint lubrication in a mammal, diminishing the action of enzymes and proinflammatory cytokines that affect joint health of a mammal.

58. The joint heath composition according to claim 44, comprising a method for improving joint movement or physical function, maintaining joint health and mobility into old age, supporting, protecting or promoting joint comfort, alleviating joint pain, reducing joint friction, alleviating joint stiffness, improving joint range of motion or flexibility, promoting mobility, reducing inflammation, reducing oxidative stress, reducing and protecting joint wear and tear, managing or treating osteoarthritis or rheumatoid arthritis, preventing osteoarthritis or rheumatoid arthritis, or reversing the progression of osteoarthritis or rheumatoid arthritis; Preventing and treating juvenile rheumatoid arthritis, Still's disease, psoriatic arthritis, reactive arthritis, septic arthritis, Reiter's syndrome, Behcet's syndrome, or Felty's syndrome or the like of a mammal.

59. The joint health composition according to claim 1, comprising a method for improving the health of joints of hand, elbow joints, wrist joints, axillary articulations, sternoclavicular joints, vertebral articulations, temporomandibular joints, sacroiliac joints, hip joints, knee joints and articulation of foot.

60. The joint health composition according to claim 24, comprising a method for improving the health of joints of hand, elbow joints, wrist joints, axillary articulations, sternoclavicular joints, vertebral articulations, temporomandibular joints, sacroiliac joints, hip joints, knee joints and articulation of foot.

61. The joint health composition according to claim 44, comprising a method for improving the health of joints of hand, elbow joints, wrist joints, axillary articulations, sternoclavicular joints, vertebral articulations, temporomandibular joints, sacroiliac joints, hip joints, knee joints and articulation of foot.
